SEQUENCE LISTING (393 sequences)

SEQ ID NO: 1 - protein, 692 aa, homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQ

SEQ ID NO: 2 - DNA, 3636 bp, homo sapiens
cagcgacgtcgaggcgctcatggttgcaggcgggcgccgccgttcagttcagggtctgagcctggaggagtgagccaggcagtgagactggctcgggcgggccgggacgcgtcgttgcagcagcggctcccagctcccagccaggattccgcgcgccccttcacgcgccctgctcctgaacttcagctcctgcacagtcctccccaccgcaaggctcaaggcgccgccggcgtggaccgcgcacggcctctaggtctcctcgccaggacagcaacctctcccctggccctcatgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaagaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtatggtcagcacactcggggcctacacggatggccacagccgtcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaaggggccgtgacagccgttgccatctgctgccggagccggcacctggcgcaggcctcccaggagctccagtgacagccccatcccaggatgggtgtctggggagggtcaagggctggggctgagctttaaaatggttccgacttgtccctctctcagccctccatggcctggcacgaggggatggggatgcttccgcctttccggggctgctggcctggcccttgagtggggcagcctccttgcctggaactcactcactctgggtgcctcctccccaggtggaggtgccaggaagctccctccctcactgtggggcatttcaccattcaaacaggtcgagctgtgctcgggtgctgccagctgctcccaatgtgccgatgtccgtgggcagaatgacttttattgagctcttgttccgtgccaggcattcaatcctcaggtctccaccaaggaggcaggattcttcccatggataggggagggggcggtaggggctgcagggacaaacatcgttggggggtgagtgtgaaaggtgctgatggccctcatctccagctaactgtggagaagcccctgggggctccctgattaatggaggcttagctttctggatggcatctagccagaggctggagacaggtgcgcccctggtggtcacaggctgtgccttggtttcctgagccacctttactctgctctatgccaggctgtgctagcaacacccaaaggtggcctgcggggagccatcacctaggactgactcggcagtgtgcagtggtgcatgcactgtctcagccaacccgctccactacccggcagggtacacattcgcacccctacttcacagaggaagaaacctggaaccagagggggcgtgcctgccaagctcacacagcaggaactgagccagaaacgcagattgggctggctctgaagccaagcctcttcttacttcacccggctgggctcctcatttttacgggtaacagtgaggctgggaaggggaacacagaccaggaagctcggtgagtgatggcagaacgatgcctgcaggcatggaactttttccgttatcacccaggcctgattcactggcctggcggagatgcttctaaggcatggtcgggggagagggccaacaactgtccctccttgagcaccagccccacccaagcaagcagacatttatcttttgggtctgtcctctctgttgcctttttacagccaacttttctagacctgttttgcttttgtaacttgaagatatttattctgggttttgtagcatttttattaatatggtgactttttaaaataaaaacaaacaaacgttgtcct

SEQ ID NO: 3 - DNA, 16 bp, artificial
  Preferred seqeunce motif
gcctgtctgtggaagc

SEQ ID NO: 4 - DNA, 16 bp, Artificial
  Preferred seqeunce motif
caagttacaaaagcaa

SEQ ID NO: 5 - DNA, 16 bp, Artificial
  Preferred seqeunce motif
gagatacacctccacc

SEQ ID NO: 6 - DNA, 16 bp, Artificial
  Preferred seqeunce motif
tcctcagggaaccagg

SEQ ID NO: 7 - DNA, 16 bp, Artificial
  Preferred seqeunce motif
ctggagcagctcagca

SEQ ID NO: 8 - DNA, 16 bp, Artificial
  Preferred seqeunce motif
catggcagcaggaagc

SEQ ID NO: 9 - DNA, 16 bp, Artificial
  LNA Oligomer
gagatacacctccacc

SEQ ID NO: 10 - DNA, 16 bp, Artificial
  LNA Oligomer
gcctgtctgtggaagc

SEQ ID NO: 11 - DNA, 16 bp, Artificial
  LNA Oligomer
caagttacaaaagcaa

SEQ ID NO: 12 - DNA, 15 bp, artificial
  Control Oligomer
cgtcagtatgcgaat

SEQ ID NO: 13 - DNA, 20 bp, Artificial
  ISIS Antisense oligonucloeitde compound
gcctcagtctgcttcgcacc

SEQ ID NO: 14 - DNA, 16 bp, homo sapiens
ggtggaggtgtatctc

SEQ ID NO: 15 - DNA, 17 bp, Homo sapiens
cgcttccacagacaggc

SEQ ID NO: 16 - DNA, 23 bp, homo sapiens
ggcctggttccctgaggaccagc

SEQ ID NO: 17 - DNA, 23 bp, homo sapiens
gaggagctgctgagctgctccag

SEQ ID NO: 18 - DNA, 23 bp, homo sapiens
gcttcctgctgccatgccccagg

SEQ ID NO: 19 - DNA, 15 bp, homo sapiens
ttgcttttgtaactt

SEQ ID NO: 20 - DNA, 14 bp, Artificial
  LNA Oligomer
gagtagaggcaggc

SEQ ID NO: 21 - DNA, 16 bp, Artificial
  LNA Oligomer
tcctcagggaaccagg

SEQ ID NO: 22 - DNA, 16 bp, Artificial
  LNA Oligomer
ctggagcagctcagca

SEQ ID NO: 23 - DNA, 16 bp, Artificial
  LNA oligomer
catggcagcaggaagc

SEQ ID NO: 24 - DNA, 14 bp, Artificial
  LNA Oligomer
gatacacctccacc

SEQ ID NO: 25 - DNA, 14 bp, Artificial
  LNA Oligomer
ctgtctgtggaagc

SEQ ID NO: 26 - DNA, 13 bp, Artificial
  LNA Oligomer
gtctgtggaagcg

SEQ ID NO: 27 - DNA, 13 bp, Artificial
  LNA Oligomer
atgagggtgccgc

SEQ ID NO: 28 - DNA, 13 bp, Artificial
  LNA Oligomer
ataaactccaggc

SEQ ID NO: 29 - DNA, 13 bp, artificial
  LNA oligomer
tagacaccctcac

SEQ ID NO: 30 - DNA, 14 bp, Artificial
  Oligomer motif
gagtagaggcaggc

SEQ ID NO: 31 - DNA, 16 bp, Artificial
  Oligomer motif
tcctcagggaaccagg

SEQ ID NO: 32 - DNA, 16 bp, Artificial
  Oligomer motif
ctggagcagctcagca

SEQ ID NO: 33 - DNA, 16 bp, Artificial
  Oligomer motif
catggcagcaggaagc

SEQ ID NO: 34 - DNA, 14 bp, Artificial
  Oligomer motif
gatacacctccacc

SEQ ID NO: 35 - DNA, 14 bp, Artificial
  Oligomer motif
ctgtctgtggaagc

SEQ ID NO: 36 - DNA, 13 bp, Artificial
  Oligomer motif
gtctgtggaagcg

SEQ ID NO: 37 - DNA, 13 bp, Artificial
  Oligomer motif
atgagggtgccgc

SEQ ID NO: 38 - DNA, 13 bp, Artificial
  Oligomer motif
ataaactccaggc

SEQ ID NO: 39 - DNA, 13 bp, Artificial
  Oligomer motif
tagacaccctcac

SEQ ID NO: 40 - DNA, 12 bp, Artificial
  Oligo motif
tctgtggaagcg

SEQ ID NO: 41 - DNA, 12 bp, Artificial
  Oligo motif
cctatgagggtg

SEQ ID NO: 42 - DNA, 12 bp, Artificial
  Oligo motif
ccgaataaactc

SEQ ID NO: 43 - DNA, 12 bp, Artificial
  Oligo motif
taaactccaggc

SEQ ID NO: 44 - DNA, 12 bp, Artificial
  Oligo motif
cggccgctgacc

SEQ ID NO: 45 - DNA, 12 bp, Artificial
  Oligo motif
ccaggcctatga

SEQ ID NO: 46 - DNA, 12 bp, Artificial
  Oligo motif
ggcctatgaggg

SEQ ID NO: 47 - DNA, 13 bp, Artificial
  Oligo motif
gtctgtggaagcg

SEQ ID NO: 48 - DNA, 13 bp, Artificial
  Oligo motif
cccggccgctgac

SEQ ID NO: 49 - DNA, 13 bp, Artificial
  Oligo motif
atgagggtgccgc

SEQ ID NO: 50 - DNA, 13 bp, Artificial
  Oligo motif
gcctatgagggtg

SEQ ID NO: 51 - DNA, 13 bp, Artificial
  Oligo motif
ccaggcctatgag

SEQ ID NO: 52 - DNA, 13 bp, Artificial
  Oligo motif
actccaggcctat

SEQ ID NO: 53 - DNA, 13 bp, Artificial
  Oligo motif
taaactccaggcc

SEQ ID NO: 54 - DNA, 13 bp, Artificial
  Oligo motif
ataaactccaggc

SEQ ID NO: 55 - DNA, 13 bp, Artificial
  Oligo motif
gccccgagtgtgc

SEQ ID NO: 56 - DNA, 13 bp, Artificial
  Oligo motif
tagacaccctcac

SEQ ID NO: 57 - DNA, 13 bp, Artificial
  Oligo motif
atggggcaacttc

SEQ ID NO: 58 - DNA, 13 bp, Artificial
  Oligo motif
gagatacacctcc

SEQ ID NO: 59 - DNA, 13 bp, Artificial
  Oligo motif
tccaggcctatga

SEQ ID NO: 60 - DNA, 13 bp, Artificial
  Oligo motif
ggccccgagtgtg

SEQ ID NO: 61 - DNA, 13 bp, Artificial
  Oligo motif
caggcctatgagg

SEQ ID NO: 62 - DNA, 13 bp, Artificial
  Oligo motif
agatacacctcca

SEQ ID NO: 63 - DNA, 13 bp, Artificial
  Oligo motif
cacgtgggcagca

SEQ ID NO: 64 - DNA, 13 bp, Artificial
  Oligo motif
tgtcacacttgct

SEQ ID NO: 65 - DNA, 13 bp, Artificial
  Oligo motif
tcccggccgctga

SEQ ID NO: 66 - DNA, 13 bp, Artificial
  Oligo motif
tatgagggtgccg

SEQ ID NO: 67 - DNA, 13 bp, Artificial
  Oligo motif
ctatgagggtgcc

SEQ ID NO: 68 - DNA, 13 bp, Artificial
  Oligo motif
cctatgagggtgc

SEQ ID NO: 69 - DNA, 13 bp, Artificial
  Oligo motif
ggcctatgagggt

SEQ ID NO: 70 - DNA, 13 bp, Artificial
  Oligo motif
aggcctatgaggg

SEQ ID NO: 71 - DNA, 13 bp, Artificial
  Oligo motif
ctccaggcctatg

SEQ ID NO: 72 - DNA, 13 bp, Artificial
  Oligo motif
tccgaataaactc

SEQ ID NO: 73 - DNA, 13 bp, Artificial
  Oligo motif
cgtcccggaagtt

SEQ ID NO: 74 - DNA, 13 bp, Artificial
  Oligo motif
taatcagggagcc

SEQ ID NO: 75 - DNA, 13 bp, Artificial
  Oligo motif
tggggcaacttca

SEQ ID NO: 76 - DNA, 13 bp, Artificial
  Oligo motif
catggggcaactt

SEQ ID NO: 77 - DNA, 13 bp, Artificial
  Oligo motif
cggccgctgacca

SEQ ID NO: 78 - DNA, 13 bp, Artificial
  Oligo motif
ccggccgctgacc

SEQ ID NO: 79 - DNA, 13 bp, Artificial
  Oligo motif
ccgaataaactcc

SEQ ID NO: 80 - DNA, 13 bp, Artificial
  Oligo motif
gtcccactctgtg

SEQ ID NO: 81 - DNA, 13 bp, Artificial
  Oligo motif
caggttgggggtc

SEQ ID NO: 82 - DNA, 13 bp, Artificial
  Oligo motif
cggcagcagatgg

SEQ ID NO: 83 - DNA, 13 bp, Artificial
  Oligo motif
acaccctcacccc

SEQ ID NO: 84 - DNA, 13 bp, Artificial
  Oligo motif
tccggcagcagat

SEQ ID NO: 85 - DNA, 13 bp, Artificial
  Oligo motif
atacacctccacc

SEQ ID NO: 86 - DNA, 13 bp, Artificial
  Oligo motif
cctgtctgtggaa

SEQ ID NO: 87 - DNA, 13 bp, Artificial
  Oligo motif
gcctgtctgtgga

SEQ ID NO: 88 - DNA, 13 bp, Artificial
  Oligo motif
ttccgaataaact

SEQ ID NO: 89 - DNA, 13 bp, Artificial
  Oligo motif
actgtgatgacct

SEQ ID NO: 90 - DNA, 13 bp, Artificial
  Oligo motif
tcgtcccggaagt

SEQ ID NO: 91 - DNA, 13 bp, Artificial
  Oligo motif
tcccactctgtga

SEQ ID NO: 92 - DNA, 13 bp, Artificial
  Oligo motif
aataaactccagg

SEQ ID NO: 93 - DNA, 13 bp, Artificial
  Oligo motif
gctggggagtaga

SEQ ID NO: 94 - DNA, 13 bp, Artificial
  Oligo motif
ttaatcagggagc

SEQ ID NO: 95 - DNA, 14 bp, Artificial
  Oligo motif
tgtctgtggaagcg

SEQ ID NO: 96 - DNA, 14 bp, Artificial
  Oligo motif
cctgtctgtggaag

SEQ ID NO: 97 - DNA, 14 bp, Artificial
  Oligo motif
ctgtcacacttgct

SEQ ID NO: 98 - DNA, 14 bp, Artificial
  Oligo motif
cggccgctgaccac

SEQ ID NO: 99 - DNA, 14 bp, Artificial
  Oligo motif
cccggccgctgacc

SEQ ID NO: 100 - DNA, 14 bp, Artificial
  Oligo motif
tcccggccgctgac

SEQ ID NO: 101 - DNA, 14 bp, Artificial
  Oligo motif
atcccggccgctga

SEQ ID NO: 102 - DNA, 14 bp, Artificial
  Oligo motif
atgagggtgccgct

SEQ ID NO: 103 - DNA, 14 bp, Artificial
  Oligo motif
tatgagggtgccgc

SEQ ID NO: 104 - DNA, 14 bp, Artificial
  Oligo motif
gcctatgagggtgc

SEQ ID NO: 105 - DNA, 14 bp, Artificial
  Oligo motif
ggcctatgagggtg

SEQ ID NO: 106 - DNA, 14 bp, Artificial
  Oligo motif
caggcctatgaggg

SEQ ID NO: 107 - DNA, 14 bp, Artificial
  Oligo motif
ccaggcctatgagg

SEQ ID NO: 108 - DNA, 14 bp, Artificial
  Oligo motif
tccaggcctatgag

SEQ ID NO: 109 - DNA, 14 bp, Artificial
  Oligo motif
ctccaggcctatga

SEQ ID NO: 110 - DNA, 14 bp, Artificial
  Oligo motif
actccaggcctatg

SEQ ID NO: 111 - DNA, 14 bp, Artificial
  Oligo motif
aactccaggcctat

SEQ ID NO: 112 - DNA, 14 bp, Artificial
  Oligo motif
aaactccaggccta

SEQ ID NO: 113 - DNA, 14 bp, Artificial
  Oligo motif
taaactccaggcct

SEQ ID NO: 114 - DNA, 14 bp, Artificial
  Oligo motif
ataaactccaggcc

SEQ ID NO: 115 - DNA, 14 bp, Artificial
  Oligo motif
aataaactccaggc

SEQ ID NO: 116 - DNA, 14 bp, Artificial
  Oligo motif
ttccgaataaactc

SEQ ID NO: 117 - DNA, 14 bp, Artificial
  Oligo motif
tcgtcccggaagtt

SEQ ID NO: 118 - DNA, 14 bp, Artificial
  Oligo motif
gagtagaggcaggc

SEQ ID NO: 119 - DNA, 14 bp, Artificial
  Oligo motif
ggggagtagaggca

SEQ ID NO: 120 - DNA, 14 bp, Artificial
  Oligo motif
gctggggagtagag

SEQ ID NO: 121 - DNA, 14 bp, Artificial
  Oligo motif
actgtgatgacctc

SEQ ID NO: 122 - DNA, 14 bp, Artificial
  Oligo motif
gtcccactctgtga

SEQ ID NO: 123 - DNA, 14 bp, Artificial
  Oligo motif
ccaggttgggggtc

SEQ ID NO: 124 - DNA, 14 bp, Artificial
  Oligo motif
ggccccgagtgtgc

SEQ ID NO: 125 - DNA, 14 bp, Artificial
  Oligo motif
acaccctcaccccc

SEQ ID NO: 126 - DNA, 14 bp, Artificial
  Oligo motif
gacaccctcacccc

SEQ ID NO: 127 - DNA, 14 bp, Artificial
  Oligo motif
tagacaccctcacc

SEQ ID NO: 128 - DNA, 14 bp, Artificial
  Oligo motif
ggggcatggcagca

SEQ ID NO: 129 - DNA, 14 bp, Artificial
  Oligo motif
cggcagcagatggc

SEQ ID NO: 130 - DNA, 14 bp, Artificial
  Oligo motif
ccggcagcagatgg

SEQ ID NO: 131 - DNA, 14 bp, Artificial
  Oligo motif
gctccggcagcaga

SEQ ID NO: 132 - DNA, 14 bp, Artificial
  Oligo motif
taatcagggagccc

SEQ ID NO: 133 - DNA, 14 bp, Artificial
  Oligo motif
ttaatcagggagcc

SEQ ID NO: 134 - DNA, 14 bp, Artificial
  Oligo motif
atggggcaacttca

SEQ ID NO: 135 - DNA, 14 bp, Artificial
  Oligo motif
catggggcaacttc

SEQ ID NO: 136 - DNA, 14 bp, Artificial
  Oligo motif
gatacacctccacc

SEQ ID NO: 137 - DNA, 14 bp, Artificial
  Oligo motif
agatacacctccac

SEQ ID NO: 138 - DNA, 14 bp, Artificial
  Oligo motif
gagatacacctcca

SEQ ID NO: 139 - DNA, 14 bp, Artificial
  Oligo motif
ctgtctgtggaagc

SEQ ID NO: 140 - DNA, 14 bp, Artificial
  Oligo motif
gcctgtctgtggaa

SEQ ID NO: 141 - DNA, 14 bp, Artificial
  Oligo motif
acgtgggcagcagc

SEQ ID NO: 142 - DNA, 14 bp, Artificial
  Oligo motif
tcctcagggaacca

SEQ ID NO: 143 - DNA, 14 bp, Artificial
  Oligo motif
gcatggcagcagga

SEQ ID NO: 144 - DNA, 14 bp, Artificial
  Oligo motif
ctccggcagcagat

SEQ ID NO: 145 - DNA, 14 bp, Artificial
  Oligo motif
cacgtgggcagcag

SEQ ID NO: 146 - DNA, 14 bp, Artificial
  Oligo motif
ccacgtgggcagca

SEQ ID NO: 147 - DNA, 14 bp, Artificial
  Oligo motif
ggagcagctcagca

SEQ ID NO: 148 - DNA, 14 bp, Artificial
  Oligo motif
gagcagctcagcag

SEQ ID NO: 149 - DNA, 14 bp, Artificial
  Oligo motif
atgggtgctggggg

SEQ ID NO: 150 - DNA, 14 bp, Artificial
  Oligo motif
ccggccgctgacca

SEQ ID NO: 151 - DNA, 14 bp, Artificial
  Oligo motif
catcccggccgctg

SEQ ID NO: 152 - DNA, 14 bp, Artificial
  Oligo motif
ctatgagggtgccg

SEQ ID NO: 153 - DNA, 14 bp, Artificial
  Oligo motif
cctatgagggtgcc

SEQ ID NO: 154 - DNA, 14 bp, Artificial
  Oligo motif
aggcctatgagggt

SEQ ID NO: 155 - DNA, 14 bp, Artificial
  Oligo motif
gaataaactccagg

SEQ ID NO: 156 - DNA, 14 bp, Artificial
  Oligo motif
cgaataaactccag

SEQ ID NO: 157 - DNA, 14 bp, Artificial
  Oligo motif
tccgaataaactcc

SEQ ID NO: 158 - DNA, 14 bp, Artificial
  Oligo motif
tccccaaagtcccc

SEQ ID NO: 159 - DNA, 14 bp, Artificial
  Oligo motif
cccactctgtgaca

SEQ ID NO: 160 - DNA, 14 bp, Artificial
  Oligo motif
agagaagtggatca

SEQ ID NO: 161 - DNA, 14 bp, Artificial
  Oligo motif
tcagggaaccaggc

SEQ ID NO: 162 - DNA, 14 bp, Artificial
  Oligo motif
accaggttgggggt

SEQ ID NO: 163 - DNA, 14 bp, Artificial
  Oligo motif
accctcacccccaa

SEQ ID NO: 164 - DNA, 14 bp, Artificial
  Oligo motif
ggtggcagtggaca

SEQ ID NO: 165 - DNA, 14 bp, Artificial
  Oligo motif
tggtggcagtggac

SEQ ID NO: 166 - DNA, 14 bp, Artificial
  Oligo motif
cctggggcatggca

SEQ ID NO: 167 - DNA, 14 bp, Artificial
  Oligo motif
tcaagttacaaaag

SEQ ID NO: 168 - DNA, 14 bp, Artificial
  Oligo motif
cccggcgggcagcc

SEQ ID NO: 169 - DNA, 14 bp, Artificial
  Oligo motif
acatggggcaactt

SEQ ID NO: 170 - DNA, 14 bp, Artificial
  Oligo motif
gtgcccttcccttg

SEQ ID NO: 171 - DNA, 14 bp, Artificial
  Oligo motif
ccgaataaactcca

SEQ ID NO: 172 - DNA, 14 bp, Artificial
  Oligo motif
ctggggagtagagg

SEQ ID NO: 173 - DNA, 14 bp, Artificial
  Oligo motif
tcccactctgtgac

SEQ ID NO: 174 - DNA, 14 bp, Artificial
  Oligo motif
gtgggcagcagcct

SEQ ID NO: 175 - DNA, 14 bp, Artificial
  Oligo motif
gagaagtggatcag

SEQ ID NO: 176 - DNA, 14 bp, Artificial
  Oligo motif
gtcctcagggaacc

SEQ ID NO: 177 - DNA, 14 bp, Artificial
  Oligo motif
caggttgggggtca

SEQ ID NO: 178 - DNA, 14 bp, Artificial
  Oligo motif
ccctcacccccaaa

SEQ ID NO: 179 - DNA, 14 bp, Artificial
  Oligo motif
ggcatggcagcagg

SEQ ID NO: 180 - DNA, 14 bp, Artificial
  Oligo motif
tggggcatggcagc

SEQ ID NO: 181 - DNA, 14 bp, Artificial
  Oligo motif
tccggcagcagatg

SEQ ID NO: 182 - DNA, 14 bp, Artificial
  Oligo motif
caagttacaaaagc

SEQ ID NO: 183 - DNA, 14 bp, Artificial
  Oligo motif
gggatgctctgggc

SEQ ID NO: 184 - DNA, 14 bp, Artificial
  Oligo motif
cgctccaggttcca

SEQ ID NO: 185 - DNA, 14 bp, Artificial
  Oligo motif
gggcagcagcctgt

SEQ ID NO: 186 - DNA, 14 bp, Artificial
  Oligo motif
catggcagcaggaa

SEQ ID NO: 187 - DNA, 14 bp, Artificial
  Oligo motif
catgggtgctgggg

SEQ ID NO: 188 - DNA, 14 bp, Artificial
  Oligo motif
gcatcccggccgct

SEQ ID NO: 189 - DNA, 14 bp, Artificial
  Oligo motif
gccacgtgggcagc

SEQ ID NO: 190 - DNA, 14 bp, Artificial
  Oligo motif
agacaccctcaccc

SEQ ID NO: 191 - DNA, 15 bp, Artificial
  Oligo motif
ctgtctgtggaagcg

SEQ ID NO: 192 - DNA, 15 bp, Artificial
  Oligo motif
cctgtctgtggaagc

SEQ ID NO: 193 - DNA, 15 bp, Artificial
  Oligo motif
gcctgtctgtggaag

SEQ ID NO: 194 - DNA, 15 bp, Artificial
  Oligo motif
ccggccgctgaccac

SEQ ID NO: 195 - DNA, 15 bp, Artificial
  Oligo motif
cccggccgctgacca

SEQ ID NO: 196 - DNA, 15 bp, Artificial
  Oligo motif
tcccggccgctgacc

SEQ ID NO: 197 - DNA, 15 bp, Artificial
  Oligo motif
atcccggccgctgac

SEQ ID NO: 198 - DNA, 15 bp, Artificial
  Oligo motif
catcccggccgctga

SEQ ID NO: 199 - DNA, 15 bp, Artificial
  Oligo motif
tatgagggtgccgct

SEQ ID NO: 200 - DNA, 15 bp, Artificial
  Oligo motif
ctatgagggtgccgc

SEQ ID NO: 201 - DNA, 15 bp, Artificial
  Oligo motif
gcctatgagggtgcc

SEQ ID NO: 202 - DNA, 15 bp, Artificial
  Oligo motif
ggcctatgagggtgc

SEQ ID NO: 203 - DNA, 15 bp, Artificial
  Oligo motif
aggcctatgagggtg

SEQ ID NO: 204 - DNA, 15 bp, Artificial
  Oligo motif
caggcctatgagggt

SEQ ID NO: 205 - DNA, 15 bp, Artificial
  Oligo motif
ccaggcctatgaggg

SEQ ID NO: 206 - DNA, 15 bp, Artificial
  Oligo motif
tccaggcctatgagg

SEQ ID NO: 207 - DNA, 15 bp, Artificial
  Oligo motif
ctccaggcctatgag

SEQ ID NO: 208 - DNA, 15 bp, Artificial
  Oligo motif
actccaggcctatga

SEQ ID NO: 209 - DNA, 15 bp, Artificial
  Oligo motif
aactccaggcctatg

SEQ ID NO: 210 - DNA, 15 bp, Artificial
  Oligo motif
aaactccaggcctat

SEQ ID NO: 211 - DNA, 15 bp, Artificial
  Oligo motif
taaactccaggccta

SEQ ID NO: 212 - DNA, 15 bp, Artificial
  Oligo motif
ataaactccaggcct

SEQ ID NO: 213 - DNA, 15 bp, Artificial
  Oligo motif
aataaactccaggcc

SEQ ID NO: 214 - DNA, 15 bp, Artificial
  Oligo motif
gaataaactccaggc

SEQ ID NO: 215 - DNA, 15 bp, Artificial
  Oligo motif
cgaataaactccagg

SEQ ID NO: 216 - DNA, 15 bp, Artificial
  Oligo motif
ttccgaataaactcc

SEQ ID NO: 217 - DNA, 15 bp, Artificial
  Oligo motif
ggagtagaggcaggc

SEQ ID NO: 218 - DNA, 15 bp, Artificial
  Oligo motif
ggggagtagaggcag

SEQ ID NO: 219 - DNA, 15 bp, Artificial
  Oligo motif
tggggagtagaggca

SEQ ID NO: 220 - DNA, 15 bp, Artificial
  Oligo motif
ctggggagtagaggc

SEQ ID NO: 221 - DNA, 15 bp, Artificial
  Oligo motif
gctggggagtagagg

SEQ ID NO: 222 - DNA, 15 bp, Artificial
  Oligo motif
gtccccaaagtcccc

SEQ ID NO: 223 - DNA, 15 bp, Artificial
  Oligo motif
gtcccactctgtgac

SEQ ID NO: 224 - DNA, 15 bp, Artificial
  Oligo motif
gccacgtgggcagca

SEQ ID NO: 225 - DNA, 15 bp, Artificial
  Oligo motif
agagaagtggatcag

SEQ ID NO: 226 - DNA, 15 bp, Artificial
  Oligo motif
ctcagggaaccaggc

SEQ ID NO: 227 - DNA, 15 bp, Artificial
  Oligo motif
gtcctcagggaacca

SEQ ID NO: 228 - DNA, 15 bp, Artificial
  Oligo motif
caggttgggggtcag

SEQ ID NO: 229 - DNA, 15 bp, Artificial
  Oligo motif
ccaggttgggggtca

SEQ ID NO: 230 - DNA, 15 bp, Artificial
  Oligo motif
accaggttgggggtc

SEQ ID NO: 231 - DNA, 15 bp, Artificial
  Oligo motif
atgggtgctgggggg

SEQ ID NO: 232 - DNA, 15 bp, Artificial
  Oligo motif
tggagcagctcagca

SEQ ID NO: 233 - DNA, 15 bp, Artificial
  Oligo motif
accctcacccccaaa

SEQ ID NO: 234 - DNA, 15 bp, Artificial
  Oligo motif
caccctcacccccaa

SEQ ID NO: 235 - DNA, 15 bp, Artificial
  Oligo motif
acaccctcaccccca

SEQ ID NO: 236 - DNA, 15 bp, Artificial
  Oligo motif
gacaccctcaccccc

SEQ ID NO: 237 - DNA, 15 bp, Artificial
  Oligo motif
agacaccctcacccc

SEQ ID NO: 238 - DNA, 15 bp, Artificial
  Oligo motif
tagacaccctcaccc

SEQ ID NO: 239 - DNA, 15 bp, Artificial
  Oligo motif
gcatggcagcaggaa

SEQ ID NO: 240 - DNA, 15 bp, Artificial
  Oligo motif
ggcatggcagcagga

SEQ ID NO: 241 - DNA, 15 bp, Artificial
  Oligo motif
ggggcatggcagcag

SEQ ID NO: 242 - DNA, 15 bp, Artificial
  Oligo motif
tggggcatggcagca

SEQ ID NO: 243 - DNA, 15 bp, Artificial
  Oligo motif
ctggggcatggcagc

SEQ ID NO: 244 - DNA, 15 bp, Artificial
  Oligo motif
cggcagcagatggca

SEQ ID NO: 245 - DNA, 15 bp, Artificial
  Oligo motif
ccggcagcagatggc

SEQ ID NO: 246 - DNA, 15 bp, Artificial
  Oligo motif
tccggcagcagatgg

SEQ ID NO: 247 - DNA, 15 bp, Artificial
  Oligo motif
ctccggcagcagatg

SEQ ID NO: 248 - DNA, 15 bp, Artificial
  Oligo motif
gctccggcagcagat

SEQ ID NO: 249 - DNA, 15 bp, Artificial
  Oligo motif
ggctccggcagcaga

SEQ ID NO: 250 - DNA, 15 bp, Artificial
  Oligo motif
ttaatcagggagccc

SEQ ID NO: 251 - DNA, 15 bp, Artificial
  Oligo motif
tcaagttacaaaagc

SEQ ID NO: 252 - DNA, 15 bp, Artificial
  Oligo motif
catggggcaacttca

SEQ ID NO: 253 - DNA, 15 bp, Artificial
  Oligo motif
acatggggcaacttc

SEQ ID NO: 254 - DNA, 15 bp, Artificial
  Oligo motif
agatacacctccacc

SEQ ID NO: 255 - DNA, 15 bp, Artificial
  Oligo motif
gagatacacctccac

SEQ ID NO: 256 - DNA, 15 bp, Artificial
  Oligo motif
tgggcagcagcctgt

SEQ ID NO: 257 - DNA, 15 bp, Artificial
  Oligo motif
acgtgggcagcagcc

SEQ ID NO: 258 - DNA, 15 bp, Artificial
  Oligo motif
cacgtgggcagcagc

SEQ ID NO: 259 - DNA, 15 bp, Artificial
  Oligo motif
tcctcagggaaccag

SEQ ID NO: 260 - DNA, 15 bp, Artificial
  Oligo motif
catgggtgctggggg

SEQ ID NO: 261 - DNA, 15 bp, Artificial
  Oligo motif
gcagctcagcagctc

SEQ ID NO: 262 - DNA, 15 bp, Artificial
  Oligo motif
gagcagctcagcagc

SEQ ID NO: 263 - DNA, 15 bp, Artificial
  Oligo motif
ggagcagctcagcag

SEQ ID NO: 264 - DNA, 15 bp, Artificial
  Oligo motif
ccacgtgggcagcag

SEQ ID NO: 265 - DNA, 15 bp, Artificial
  Oligo motif
gctggtcctcaggga

SEQ ID NO: 266 - DNA, 15 bp, Artificial
  Oligo motif
tgggtgctggggggc

SEQ ID NO: 267 - DNA, 15 bp, Artificial
  Oligo motif
cctgccaggtgggtg

SEQ ID NO: 268 - DNA, 15 bp, Artificial
  Oligo motif
gcatcccggccgctg

SEQ ID NO: 269 - DNA, 15 bp, Artificial
  Oligo motif
cctatgagggtgccg

SEQ ID NO: 270 - DNA, 15 bp, Artificial
  Oligo motif
ccgaataaactccag

SEQ ID NO: 271 - DNA, 15 bp, Artificial
  Oligo motif
tccgaataaactcca

SEQ ID NO: 272 - DNA, 15 bp, Artificial
  Oligo motif
tccccaaagtcccca

SEQ ID NO: 273 - DNA, 15 bp, Artificial
  Oligo motif
tcccactctgtgaca

SEQ ID NO: 274 - DNA, 15 bp, Artificial
  Oligo motif
ggcagcagcctgtga

SEQ ID NO: 275 - DNA, 15 bp, Artificial
  Oligo motif
cgtgggcagcagcct

SEQ ID NO: 276 - DNA, 15 bp, Artificial
  Oligo motif
tcagggaaccaggcc

SEQ ID NO: 277 - DNA, 15 bp, Artificial
  Oligo motif
cctcagggaaccagg

SEQ ID NO: 278 - DNA, 15 bp, Artificial
  Oligo motif
caccaggttgggggt

SEQ ID NO: 279 - DNA, 15 bp, Artificial
  Oligo motif
tggtggcagtggaca

SEQ ID NO: 280 - DNA, 15 bp, Artificial
  Oligo motif
catggcagcaggaag

SEQ ID NO: 281 - DNA, 15 bp, Artificial
  Oligo motif
gggcatggcagcagg

SEQ ID NO: 282 - DNA, 15 bp, Artificial
  Oligo motif
cctggggcatggcag

SEQ ID NO: 283 - DNA, 15 bp, Artificial
  Oligo motif
gccggctccggcagc

SEQ ID NO: 284 - DNA, 15 bp, Artificial
  Oligo motif
ccccggcgggcagcc

SEQ ID NO: 285 - DNA, 15 bp, Artificial
  Oligo motif
ctgccaggtgggtgc

SEQ ID NO: 286 - DNA, 15 bp, Artificial
  Oligo motif
gggcagcagcctgtg

SEQ ID NO: 287 - DNA, 15 bp, Artificial
  Oligo motif
gtgggcagcagcctg

SEQ ID NO: 288 - DNA, 15 bp, Artificial
  Oligo motif
ggtcctcagggaacc

SEQ ID NO: 289 - DNA, 15 bp, Artificial
  Oligo motif
tggtcctcagggaac

SEQ ID NO: 290 - DNA, 15 bp, Artificial
  Oligo motif
gccaccaggttgggg

SEQ ID NO: 291 - DNA, 15 bp, Artificial
  Oligo motif
cagctcagcagctcc

SEQ ID NO: 292 - DNA, 15 bp, Artificial
  Oligo motif
agctgcagcctgtga

SEQ ID NO: 293 - DNA, 15 bp, Artificial
  Oligo motif
ccggctccggcagca

SEQ ID NO: 294 - DNA, 15 bp, Artificial
  Oligo motif
caagttacaaaagca

SEQ ID NO: 295 - DNA, 15 bp, Artificial
  Oligo motif
gggagtagaggcagg

SEQ ID NO: 296 - DNA, 15 bp, Artificial
  Oligo motif
agcagctcagcagct

SEQ ID NO: 297 - DNA, 15 bp, Artificial
  Oligo motif
atggcagcaggaagc

SEQ ID NO: 298 - DNA, 15 bp, Artificial
  Oligo motif
tgccaggtgggtgcc

SEQ ID NO: 299 - DNA, 16 bp, Artificial
  Oligo motif
cctgtctgtggaagcg

SEQ ID NO: 300 - DNA, 16 bp, Artificial
  Oligo motif
gcctgtctgtggaagc

SEQ ID NO: 301 - DNA, 16 bp, Artificial
  Oligo motif
cccggccgctgaccac

SEQ ID NO: 302 - DNA, 16 bp, Artificial
  Oligo motif
tcccggccgctgacca

SEQ ID NO: 303 - DNA, 16 bp, Artificial
  Oligo motif
atcccggccgctgacc

SEQ ID NO: 304 - DNA, 16 bp, Artificial
  Oligo motif
catcccggccgctgac

SEQ ID NO: 305 - DNA, 16 bp, Artificial
  Oligo motif
gcatcccggccgctga

SEQ ID NO: 306 - DNA, 16 bp, Artificial
  Oligo motif
ctatgagggtgccgct

SEQ ID NO: 307 - DNA, 16 bp, Artificial
  Oligo motif
cctatgagggtgccgc

SEQ ID NO: 308 - DNA, 16 bp, Artificial
  Oligo motif
gcctatgagggtgccg

SEQ ID NO: 309 - DNA, 16 bp, Artificial
  Oligo motif
ggcctatgagggtgcc

SEQ ID NO: 310 - DNA, 16 bp, Artificial
  Oligo motif
aggcctatgagggtgc

SEQ ID NO: 311 - DNA, 16 bp, Artificial
  Oligo motif
caggcctatgagggtg

SEQ ID NO: 312 - DNA, 16 bp, Artificial
  Oligo motif
ccaggcctatgagggt

SEQ ID NO: 313 - DNA, 16 bp, Artificial
  Oligo motif
tccaggcctatgaggg

SEQ ID NO: 314 - DNA, 16 bp, Artificial
  Oligo motif
ctccaggcctatgagg

SEQ ID NO: 315 - DNA, 16 bp, Artificial
  Oligo motif
actccaggcctatgag

SEQ ID NO: 316 - DNA, 16 bp, Artificial
  Oligo motif
aactccaggcctatga

SEQ ID NO: 317 - DNA, 16 bp, Artificial
  Oligo motif
aaactccaggcctatg

SEQ ID NO: 318 - DNA, 16 bp, Artificial
  Oligo motif
taaactccaggcctat

SEQ ID NO: 319 - DNA, 16 bp, Artificial
  Oligo motif
ataaactccaggccta

SEQ ID NO: 320 - DNA, 16 bp, Artificial
  Oligo motif
aataaactccaggcct

SEQ ID NO: 321 - DNA, 16 bp, Artificial
  Oligo motif
gaataaactccaggcc

SEQ ID NO: 322 - DNA, 16 bp, Artificial
  Oligo motif
cgaataaactccaggc

SEQ ID NO: 323 - DNA, 16 bp, Artificial
  Oligo motif
ccgaataaactccagg

SEQ ID NO: 324 - DNA, 16 bp, Artificial
  Oligo motif
ttccgaataaactcca

SEQ ID NO: 325 - DNA, 16 bp, Artificial
  Oligo motif
gggagtagaggcaggc

SEQ ID NO: 326 - DNA, 16 bp, Artificial
  Oligo motif
ggggagtagaggcagg

SEQ ID NO: 327 - DNA, 16 bp, Artificial
  Oligo motif
tggggagtagaggcag

SEQ ID NO: 328 - DNA, 16 bp, Artificial
  Oligo motif
ctggggagtagaggca

SEQ ID NO: 329 - DNA, 16 bp, Artificial
  Oligo motif
gctggggagtagaggc

SEQ ID NO: 330 - DNA, 16 bp, Artificial
  Oligo motif
gtccccaaagtcccca

SEQ ID NO: 331 - DNA, 16 bp, Artificial
  Oligo motif
gtcccactctgtgaca

SEQ ID NO: 332 - DNA, 16 bp, Artificial
  Oligo motif
gggcagcagcctgtga

SEQ ID NO: 333 - DNA, 16 bp, Artificial
  Oligo motif
tgggcagcagcctgtg

SEQ ID NO: 334 - DNA, 16 bp, Artificial
  Oligo motif
gtgggcagcagcctgt

SEQ ID NO: 335 - DNA, 16 bp, Artificial
  Oligo motif
acgtgggcagcagcct

SEQ ID NO: 336 - DNA, 16 bp, Artificial
  Oligo motif
gccacgtgggcagcag

SEQ ID NO: 337 - DNA, 16 bp, Artificial
  Oligo motif
ctcagggaaccaggcc

SEQ ID NO: 338 - DNA, 16 bp, Artificial
  Oligo motif
cctcagggaaccaggc

SEQ ID NO: 339 - DNA, 16 bp, Artificial
  Oligo motif
tcctcagggaaccagg

SEQ ID NO: 340 - DNA, 16 bp, Artificial
  Oligo motif
gtcctcagggaaccag

SEQ ID NO: 341 - DNA, 16 bp, Artificial
  Oligo motif
ggtcctcagggaacca

SEQ ID NO: 342 - DNA, 16 bp, Artificial
  Oligo motif
gctggtcctcagggaa

SEQ ID NO: 343 - DNA, 16 bp, Artificial
  Oligo motif
ccaggttgggggtcag

SEQ ID NO: 344 - DNA, 16 bp, Artificial
  Oligo motif
accaggttgggggtca

SEQ ID NO: 345 - DNA, 16 bp, Artificial
  Oligo motif
caccaggttgggggtc

SEQ ID NO: 346 - DNA, 16 bp, Artificial
  Oligo motif
ccaccaggttgggggt

SEQ ID NO: 347 - DNA, 16 bp, Artificial
  Oligo motif
tgggtgctggggggca

SEQ ID NO: 348 - DNA, 16 bp, Artificial
  Oligo motif
atgggtgctggggggc

SEQ ID NO: 349 - DNA, 16 bp, Artificial
  Oligo motif
catgggtgctgggggg

SEQ ID NO: 350 - DNA, 16 bp, Artificial
  Oligo motif
gcagctcagcagctcc

SEQ ID NO: 351 - DNA, 16 bp, Artificial
  Oligo motif
agcagctcagcagctc

SEQ ID NO: 352 - DNA, 16 bp, Artificial
  Oligo motif
gagcagctcagcagct

SEQ ID NO: 353 - DNA, 16 bp, Artificial
  Oligo motif
ggagcagctcagcagc

SEQ ID NO: 354 - DNA, 16 bp, Artificial
  Oligo motif
tggagcagctcagcag

SEQ ID NO: 355 - DNA, 16 bp, Artificial
  Oligo motif
ctggagcagctcagca

SEQ ID NO: 356 - DNA, 16 bp, Artificial
  Oligo motif
caccctcacccccaaa

SEQ ID NO: 357 - DNA, 16 bp, Artificial
  Oligo motif
acaccctcacccccaa

SEQ ID NO: 358 - DNA, 16 bp, Artificial
  Oligo motif
gacaccctcaccccca

SEQ ID NO: 359 - DNA, 16 bp, Artificial
  Oligo motif
agacaccctcaccccc

SEQ ID NO: 360 - DNA, 16 bp, Artificial
  Oligo motif
tagacaccctcacccc

SEQ ID NO: 361 - DNA, 16 bp, Artificial
  Oligo motif
catggcagcaggaagc

SEQ ID NO: 362 - DNA, 16 bp, Artificial
  Oligo motif
gcatggcagcaggaag

SEQ ID NO: 363 - DNA, 16 bp, Artificial
  Oligo motif
ggcatggcagcaggaa

SEQ ID NO: 364 - DNA, 16 bp, Artificial
  Oligo motif
gggcatggcagcagga

SEQ ID NO: 365 - DNA, 16 bp, Artificial
  Oligo motif
ggggcatggcagcagg

SEQ ID NO: 366 - DNA, 16 bp, Artificial
  Oligo motif
tggggcatggcagcag

SEQ ID NO: 367 - DNA, 16 bp, Artificial
  Oligo motif
ctggggcatggcagca

SEQ ID NO: 368 - DNA, 16 bp, Artificial
  Oligo motif
cctggggcatggcagc

SEQ ID NO: 369 - DNA, 16 bp, Artificial
  Oligo motif
ccggcagcagatggca

SEQ ID NO: 370 - DNA, 16 bp, Artificial
  Oligo motif
tccggcagcagatggc

SEQ ID NO: 371 - DNA, 16 bp, Artificial
  Oligo motif
ctccggcagcagatgg

SEQ ID NO: 372 - DNA, 16 bp, Artificial
  Oligo motif
gctccggcagcagatg

SEQ ID NO: 373 - DNA, 16 bp, Artificial
  Oligo motif
ggctccggcagcagat

SEQ ID NO: 374 - DNA, 16 bp, Artificial
  Oligo motif
cggctccggcagcaga

SEQ ID NO: 375 - DNA, 16 bp, Artificial
  Oligo motif
gccggctccggcagca

SEQ ID NO: 376 - DNA, 16 bp, Artificial
  Oligo motif
caagttacaaaagcaa

SEQ ID NO: 377 - DNA, 16 bp, Artificial
  Oligo motif
tcaagttacaaaagca

SEQ ID NO: 378 - DNA, 16 bp, Artificial
  Oligo motif
acatggggcaacttca

SEQ ID NO: 379 - DNA, 16 bp, Artificial
  Oligo motif
gagatacacctccacc

SEQ ID NO: 380 - DNA, 16 bp, Artificial
  Oligo motif
cacgtgggcagcagcc

SEQ ID NO: 381 - DNA, 16 bp, Artificial
  Oligo motif
ccacgtgggcagcagc

SEQ ID NO: 382 - DNA, 16 bp, Artificial
  Oligo motif
tggtcctcagggaacc

SEQ ID NO: 383 - DNA, 16 bp, Artificial
  Oligo motif
ctgccaggtgggtgcc

SEQ ID NO: 384 - DNA, 16 bp, Artificial
  Oligo motif
cctgccaggtgggtgc

SEQ ID NO: 385 - DNA, 16 bp, Artificial
  Oligo motif
tccgaataaactccag

SEQ ID NO: 386 - DNA, 16 bp, Artificial
  Oligo motif
cgtgggcagcagcctg

SEQ ID NO: 387 - DNA, 16 bp, Artificial
  Oligo motif
agctgcagcctgtgag

SEQ ID NO: 388 - DNA, 16 bp, Artificial
  Oligo motif
ccggctccggcagcag

SEQ ID NO: 389 - DNA, 16 bp, Artificial
  Oligo motif
ctggtcctcagggaac

SEQ ID NO: 390 - DNA, 16 bp, Artificial
  Oligo motif
gccaccaggttggggg

SEQ ID NO: 391 - DNA, 16 bp, Artificial
  Oligo motif
ggccaccaggttgggg

SEQ ID NO: 392 - DNA, 16 bp, Artificial
  Oligo motif
agctcagcagctcctc

SEQ ID NO: 393 - DNA, 16 bp, Artificial
  Oligo motif
cagctcagcagctcct

(19) United States
(12) Patent Application Publication
(10) Pub. No.: US 2010/0216864 A1
(43) Pub. Date: Aug. 26, 2010

(54) RNA Antagonist Compounds for the Modulation of PCSK9

(76) Inventors: Ellen Marie Straarup, Birkerod (DK); Niels Fisker Nielsen, Lyngby (DK)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 12/444,806

(22) PCT Filed: Oct. 9, 2007
(86) PCT No.: PCT/EP07/60703

§ 371 Date: Nov. 30, 2009

Related U.S. Application Data
(60) Provisional application No. 60/828,735, filed on Oct. 9, 2006.
(60) Provisional application No. 60/972,932, filed on Sep. 17, 2007.
(60) Provisional application No. 60/977,409, filed on Oct. 4, 2007.

(51) Int. Cl. A61K 31/7052; C12N 15/52; C12N 5/07; A61P 3/04; A61P 3/10

(52) U.S. Cl. 514/44 A; 536/23.2; 514/44 R; 435/375

(57) ABSTRACT

The present invention provides compounds, compositions and methods for modulating the expression of PCSK9. In particular, this invention relates to oligomeric compounds, such as oligonucleotide compounds, which are hybridisable with target nucleic acids encoding PCSK9, and methods for the preparation of such oligomeric compounds. The oligonucleotide compounds have been shown to modulate the expression of PCSK9, and pharmaceutical preparations thereof and their use as treatment of hypercholesterolemia and related disorders are disclosed.

### FIELD OF THE INVENTION

The present invention provides compounds, compositions and methods for modulating the expression of PCSK9. In particular, this invention relates to oligomeric compounds, such as oligonucleotide compounds, which are hybridisable with target nucleic acids encoding PCSK9, and methods for the preparation of such oligomeric compounds. The oligonucleotide compounds have been shown to modulate the expression of PCSK9, and pharmaceutical preparations thereof and their use as treatment of hypercholesterolemia and related disorders are disclosed.

### BACKGROUND

Proprotein convertase subtilisin/kexin type 9a (PCSK9) is a member of the proteinase K subfamily of subtilases. The PCSK9 gene (NARC-1) has been identified as a third locus involved in autosomal dominant hypercholesterolemia (ADH), characterised by high levels of low-density lipoprotein (LDL), xhantomas, and a high frequency of coronary heart disease. The other two loci being apolipoprotein-B (Apo-B) and the LDL receptor (LDLR). PCSK9 acts as a natural inhibitor of the LDL-receptor pathway, and both genes are regulated by depletion of cholesterol cell content and statins via sterol regulatory element-binding protein (SREBP). PCSK9 mRNA and protein levels are regulated by food intake, insulin and cell cholesterol levels (Costet et al., J. Biol. Chem. January 2006).

The human NARC1 mRNA (cDNA) sequence, which encodes human PCSK9 is shown as SEQ ID NO 2 (NCBI Acc. No. NM_{—}174936).

The human PCSK9 polypeptide sequence (nascent) is shown as SEQ ID NO 1 (NCBI Acc. No. NP_{—}777596. The polypeptide has a signal peptide between residues 1-30, which is co-translationally cleaved to produce a proprotein (31-692 of SEQ ID No 2), which is subsequently cleaved by a protease to produce a mature protein corresponding to amino acids 83-692 of SEQ ID NO 2. A glycosylation site has been characterised at residue 533.

Park et al., (J. Biol. Chem. 279, pp 50630-50638, 2004) discloses that over-expression of PCSK9 reduced LDLR protein resulting in an increase in plasma LDL cholesterol, and suggests that an inhibitor of PCSK9 function may increase LDLR protein levels and enhance LDL clearance from plasma.

Rashid et al., (2005, PNAS 102, No 15, pp 5374-5379) discloses that knockout mice lacking PCSK9 manifest increased LDLR protein leading to an increased clearance of circulating lipoproteins and decreased plasma cholesterol levels, and suggests that inhibitors of PCSK9 may be useful for the treatment of hypercholesterolemia and that there may be synergy between inhibitors of PCSK9 and statins to enhance LDLRs and reduce plasma cholesterol.

WO01/57081 discloses the NARC-1 polynucleotide sequence and discloses that antisense nucleic acids can be designed using the NARC-1 polynucleotide sequence, and that such antisense nucleic acids may comprise modified nucleotides or bases, such as peptide nucleic acids.

WO2004/097047, which discloses two mutants of PCSK9 which are associated with ADH, suggests that antisense or RNAi of such PCSK9 mutants may be used for treatment of ADH.

### OBJECT OF THE INVENTION

The invention provides therapeutic solutions for the treatment of hypercholesterolemia and related disorders, based upon oligomeric compounds, such as antisense oligonucleotides, targeted against PCSK9 nucleic acids. The inventors have discovered that the use of nucleotide analogues which have an enhanced affinity for their complementary binding partner, such as Locked Nucleic Acid (LNA) nucleotide analogues, within oligomeric compounds that are targeted towards PCSK9 target nucleic acids, provide highly effective modulation, particularly the down-regulation, of PCSK9 (NARC1) expression.

### SUMMARY OF THE INVENTION

The invention provides for oligomeric compounds capable of the modulation of the expression of mammalian, such as human PCSK9.

The invention provides an oligomer of between 10-50 nucleobases in length which consists or comprises a contiguous nucleobase sequence of a total of between 10-50 nucleobases, wherein said contiguous nucleobase sequence is at least 80% homologous to a corresponding region of a nucleic acid which encodes a mammalian PCSK9, such as at least 85% homologous, such as at least 90% homologous, such as at least 95% homologous, such as at least 97% homologous, such as 100% homologous (such as complementary) to the corresponding sequence present in the nucleic acid which encodes the PCSK9 polypeptide.

The invention further provides a conjugate comprising the oligomer according to the invention, such as a conjugate which, in addition to the nucleobase sequence of the oligomer comprises at least one non-nucleotide or non-polynucleotide moiety covalently attached to the oligomer of the invention.

The invention provides for a compound (such as an oligomer) consisting of a sequence of total of between 10-50, such as between 10-30 nucleobases, said compound comprises a subsequence of at least 8 contiguous nucleobases, wherein said subsequence corresponds to a contiguous sequence which is present in the naturally occurring mammalian nucleic acid which encodes a PCSK9 polypeptide, wherein said subsequence may comprise no more than one mismatch when compared to the corresponding nucleic acid which encodes the PCSK9 polypeptide.

The compound may further comprise a 5′ flanking nucleobase sequence, or a 3′ flanking sequence, or both a 5′ and a 3′ flanking sequence which is/are contiguous to said subsequence, wherein said flanking sequence or sequences consist of a total of between 2 and 42 nucleobase units, such as between 2 and 22 nucleobase units, which when combined with said sub-sequence, the combined contiguous nucleobase sequence, i.e. consisting of said subsequence and said flanking sequence or sequences, is at least 80% homologous, such as at least 85% homologous, such as at least 90% homologous, such as at least 95% homologous, such as at least 97% homologous, such as 100% homologous to the corresponding sequence present in the nucleic acid which encodes the PCSK9 polypeptide.

The invention further provides for an oligomer according to the invention, for use in medicine.

Further provided are methods of modulating the expression of PCSK9 in mammalian cells or tissues comprising contacting said mammalian cells or tissues with one or more of the oligomeric compounds or compositions of the invention. Typically the expression of PCSK9 is inhibited or reduced.

Also disclosed are methods of treating a mammal, such as a human, suspected of having or being prone to a disease or condition, associated with expression of PCSK9, such as hypercholesterolemia or related disorder, by administering a therapeutically or prophylactically effective amount of one or more of the oligomeric compounds or compositions of the invention.

Further, methods of using oligomeric compounds for the inhibition of expression of PCSK9 and for treatment of diseases associated with PCSK9 activity are provided, such as hypercholesterolemia and/or related disorders.

The invention provides for pharmaceutical composition comprising the oligomer or conjugate of the invention, and a pharmaceutically acceptable diluent, carrier, salt or adjuvant.

The invention also provides pharmaceutical compositions which comprise oligomeric compounds according to the invention and further compounds capable of modulating blood serum cholesterol levels, such as apolipoprotein B (Apo-B100) modulators, in particular antisense oligonucleotides (oligomers) targeted to Apo-B nucleic acid targets.

The invention provides for a method of (i) reducing the level of blood serum cholesterol or ii) reducing the level of blood serum LDL-cholesterol, or iii) for improving the HDL/LDL ratio, in a patient, the method comprising the step of administering the oligomer or the conjugate or the pharmaceutical composition according to the invention to the patient.

The invention provides for a method of lowering the plasma triglyceride in a patient, the method comprising the step of administering the oligomer or the conjugate or the pharmaceutical composition according to the invention to the patient so that the blood serum triglyceride level is reduced.

The invention provides for a method of treating obesity in a patient, the method comprising the step of administering the oligomer or the conjugate or the pharmaceutical composition according to the invention to the patient in need of treatment so that the body weight of the patient is reduced.

The invention provides for a method of treating hypercholesterolemia, or related disorder, in a patient, the method comprising the step of administering the oligomer or the conjugate or the pharmaceutical composition according to the invention to the patient in need of treatment for hypercholesterolemia, or related disorder.

The invention provides for a method of treating insulin resistance in a patient, the method comprising the step of administering the oligomer or the conjugate or the pharmaceutical composition according to the invention to the patient in need of treatment so that the patient's sensitivity to insulin is increased.

The invention provides for a method of treating type II diabetes in a patient, the method comprising the step of administering the oligomer or the conjugate or the pharmaceutical composition according to the invention to the patient suffering from type II diabetes.

The invention provides for a method for treating a metabolic disorder such as metabolic syndrome, diabetes or atherosclerosis, the method comprising the step of administering the oligomer or the conjugate or the pharmaceutical composition according to the invention to the patient in need thereof.

The invention provides for the oligomer or conjugate according to the invention for the treatment of a disease or disorder selected from the group consisting of: hypercholesterolemia or related disorder, an inflammatory disease or disorder, arthritis, asthma alzheimer's disease, a metabolic disease or disorder, metabolic syndrome, diabetes and atherosclerosis.

### BRIEF DESCRIPTION OF THE FIGURES

FIG. 1 shows a diagrammatic representation of the interaction of PCSK9 and the LDLr: PCSK9 alters the expression of the LDL receptor (LDLr). LDLr is expressed at the basolateral surface of hepatocytes and interacts with apoB-100, thereby allowing the uptake of plasma LDL and possibly that of nascent VLDL. The cellular internalisation of apoB-100 containing lipoproteins requires the ARH (Autosomal Recessive Hypercholesterolemia) adaptor protein. PCSK9 alters the post-translational expression of LDLr. PCSK9 and LDLr genes are upregulated upon low levels of intracellular cholesterol, indicating that both genes are indirect targets of HMGCoA reductase inhibitors (statins)—(Lambert et al. 2006, TRENDS in Endocrinology and Metabolism, 17:79-81).

FIG. 2 PCSK9mRNA expression in Huh-7 cells 24 hours after transfection with Lipofectamine and LNA oligonucleotides Compound ID NO#s: 262 or 338 at 0.04, 0.2, 1, 5, 10 or 25 nM. Data are normalised to Gapdh and presented relative to the mock control.

FIG. 3 PCSK9mRNA expression in Huh-7 cells 24 hours after transfection with Lipofectamine and LNA oligonucleotides Compound ID NO#s: 98 or 101 at 0.04, 0.2, 1, 5 or 10 nM. Data are normalised to Gapdh and presented relative to the mock control.

FIG. 4. PCSK9mRNA expression in Huh-7 cells 24 hours after transfection with Lipofectamine and LNA oligonucleotides Compound ID NO#s: 9, 16 or 18 at 0.04, 0.2, 1, 5, 10 or 25 nM. Data are normalised to Gapdh and presented relative to the mock control.

FIG. 5. In vitro results in the Murine hepatocarcinoma cell line Hepa 1-6: PCSK9mRNA expression in Huh-7 cells 24 hours after transfection with Lipofectamine and LNA oligonucleotides Compound ID NO#s: 262 and 338 at 0.04, 0.2, 1, 5, 10 or 25 nM. Data are normalised to Gapdh and presented relative to the mock control.

FIG. 6. PCSK9mRNA expression in Huh-7 cells 24 hours after transfection with Lipofectamine and LNA oligonucleotides Compound ID NO#s: 98 and 101 at 0.04, 0.2, 1, 5, 10 or 25 nM. Data are normalised to Gapdh and presented relative to the mock control.

FIG. 7. In vivo examination of LNA oligonucleotides in female C57BL/6 mice: PCSK9 mRNA expression in Liver following dosing 5, 10 or 15 mg/kg Compound ID NO#s: 98, 101 or 317 Days 0, 3, 7, 10, and 14 and. Day 16 the mice were sacrificed and the liver was examined by qPCR for PCSK9 mRNA expression. Data represent mean SD and is presented relative to the saline group.

FIG. 8. Serum total-, VLDL+LDL- and HDL cholesterol measured at sacrifice day 16 in C57BL/6 female mice dosed 10 mg/kg/dose of Compound ID NO#s: 98 or 101 at days 0, 3, 7, 10 and 14 by tail vein injections.

FIG. 9. Liver was sampled at sacrifice day 16 and analysed for LDL-receptor protein level by Western Blotting as described in example 13.

FIG. 10. NMRI female mice: PCSK9 mRNA expression in Liver following dosing 10 mg/kg of Compound ID NO#s: 98 or 101 days 0, 3, 7, 10, and 14 and. Day 16 the mice were sacrificed and the liver was examined by qPCR for PCSK9 mRNA expression. Data represent mean SD and is presented relative to the saline group.

FIG. 11. Total cholesterol in serum from blood sampled at sacrifice (day 16)

FIG. 12. Liver was sampled at sacrifice day 16 and analysed for LDL-receptor protein level by Western Blotting as described in example 13.

FIG. 13. Efficacy study in female and male C57BL/6 fe at High fat diet (HFD): PCSK9 mRNA expression in Liver following dosing 10 or 15 mg/kg of Compound ID NO#s: 98, 101 or 317 Days 0, 3, 7, 10, and 14 and. Day 16 the mice were sacrificed and the liver was examined by qPCR for PCSK9 mRNA expression. Female mice were fed a high diet (HFD) for 5 month before treatment with LNA oligonucleotides and male mice were fed HFD for one month before treatment. Data represent mean SD and is presented relative to the saline group.

FIG. 14. Liver was sampled at sacrifice day 16 and analysed for LDL-receptor protein level by Western Blotting as described in example 13.

FIG. 15. 13-mer LNA oligonucleotides tested in C57BL/6 female mice: PCSK9 mRNA expression in Liver following dosing 15 mg/kg of Compound ID NO#s: 9, 16, 18 or 98 Days 0, 2 and 4 and day 6 the mice were sacrificed and the liver was examined by qPCR for PCSK9 mRNA expression. Data are normalised to Gapdh and present relative to saline group in mean SD.

FIG. 16. The distribution of the different lipoprotein fractions HDL, VLDL and LDL in serum. The lipoproteins were separated on Sebia Gels and quantified using Sudan Black staining and Densiometric analysis (Molecular Imager FX). Data are presented as mean SD, n=5.

FIG. 17 shows a Clustal W local sequence alignment between the human NM_{—}174936) and the mouse (NM_{—}153565) PCSK9 encoding nucleic acids and illustrates regions where there are sufficient sequence homology to design oligomeric compounds which are complementary to both the human and mouse PCSK9 target nucleic acids, (illustrated by the vertical lines between the aligned nucleotides) shaded areas indicate preferred regions for targeting oligonucleotides to (preferably at a contiguous series of at least 12 conserved residues) both human and mouse PCSK9 activity, the underlined regions are regions which are particularly preferred.

### RELATED CASES

This case claims priority from U.S. provisional application 60/828,735 and U.S. 60/972,932, which are hereby incorporated by reference,

Furthermore, this case claims priority from U.S. 60/977,409, which is hereby incorporated by reference.

### DESCRIPTION OF THE INVENTION

### Oligomers Targeting PCSK9

The present invention employs oligomeric compounds (referred to as oligomers herein), particularly antisense oligonucleotides, for use in modulating the function of nucleic acid molecules encoding mammalian PCSK9, such as the PCSK9 protein shown in SEQ ID NO 1, and naturally occurring allelic variants of such nucleic acid molecules encoding mammalian PCSK9.

In one embodiment, the compound is at least 80% homologous to a corresponding nucleic acid which encodes a mammalian PCSK9, such as at least 85%, 90%, 91%, 92%, 93%, 93⅓%, 93.75%, 94%, 95%, 96% or at least 97% complementary, such at least 98% complementary. such as 100% complementary to the corresponding region (such as the sense or preferably the antisense strand) of the nucleic acid target sequence, such as the mRNA which encodes the PCSK9 polypeptide, such as SEQ ID NO 2, or naturally occurring allelic variants thereof.

The mammalian PCSK9 is preferably selected for the group consisting of primate, human, monkey, chimpanzee; rodent, rat, mouse, and rabbit; preferably the mammalian PCSK9 is human PCSK9.

The oligomer typically comprises or consists of a contiguous nucleobase sequence.

In one embodiment, the nucleobase sequence of the oligomer consists of the contiguous nucleobase sequence.

### Sub-Sequences and Flanking Sequences

In one embodiment, the oligomeric compound comprises at least a core sub-sequence of at least 8, such as at least 10, such as at least 12, such as at least 13, such as at least 14 contiguous nucleobases, wherein said subsequence corresponds to a contiguous sequence which is present in the naturally occurring mammalian nucleic acid which encodes a PCSK9 polypeptide, such as the human PCSK9, the cDNA sequence is illustrated as SEQ ID NO 2, wherein said subsequence may comprise no more than one mismatch when compared to the corresponding mammalian nucleic acid.

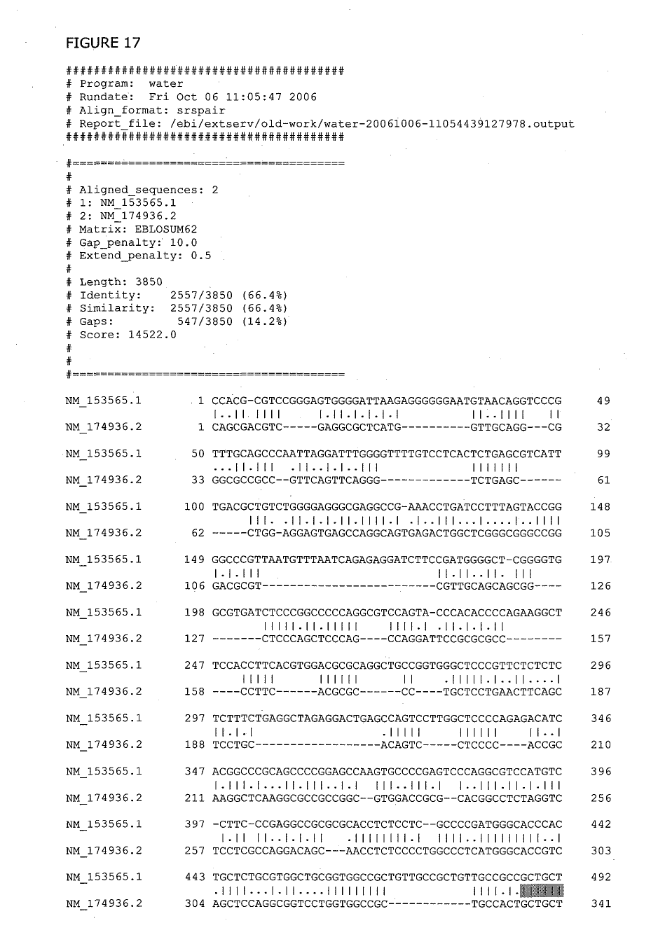

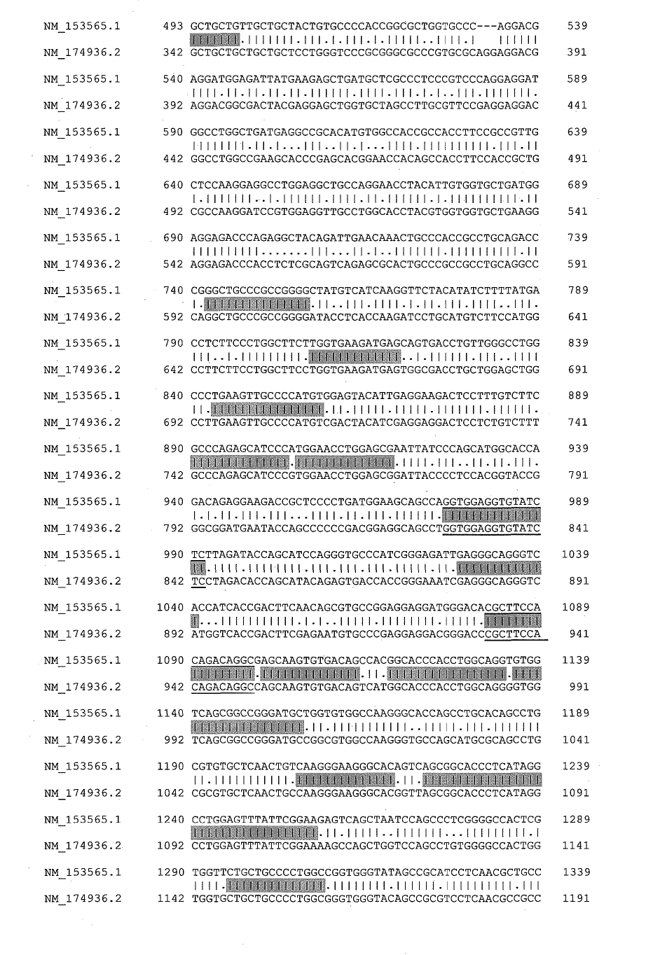

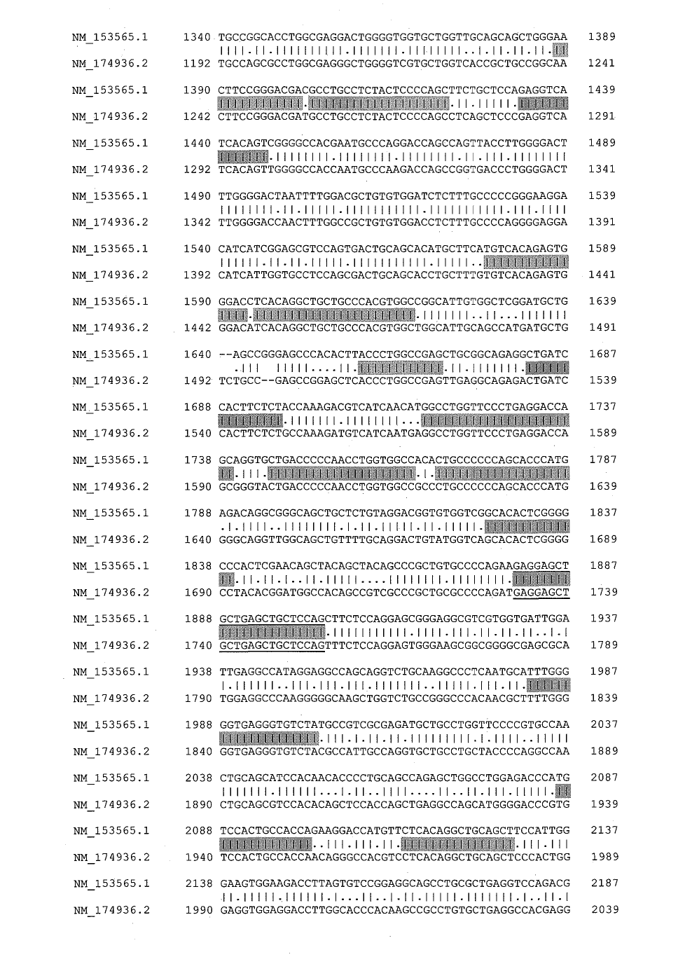

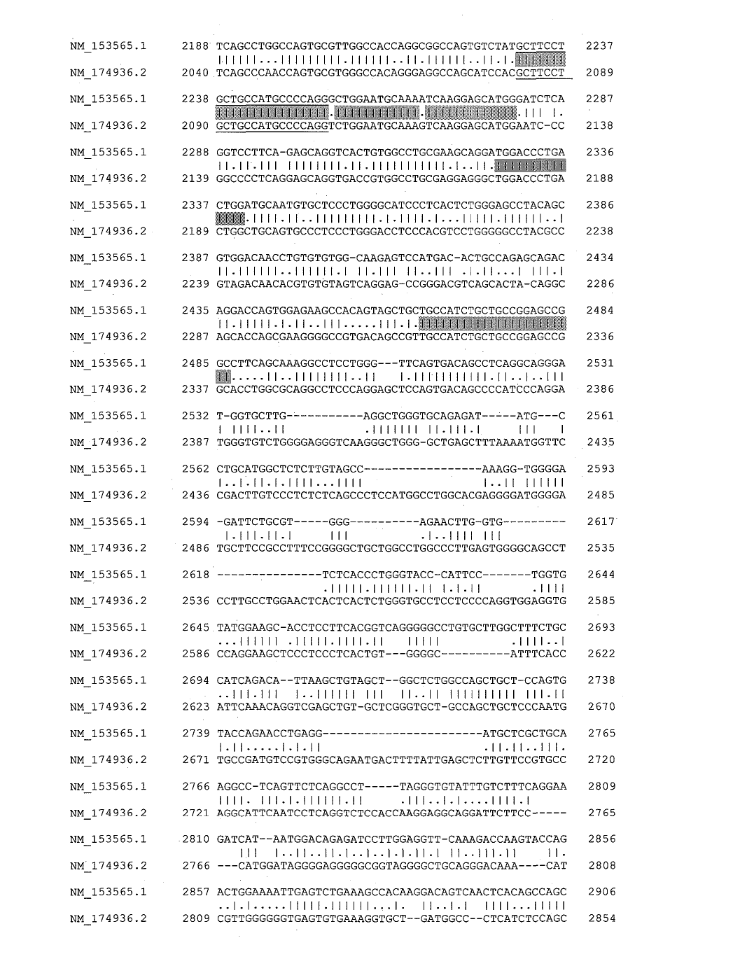

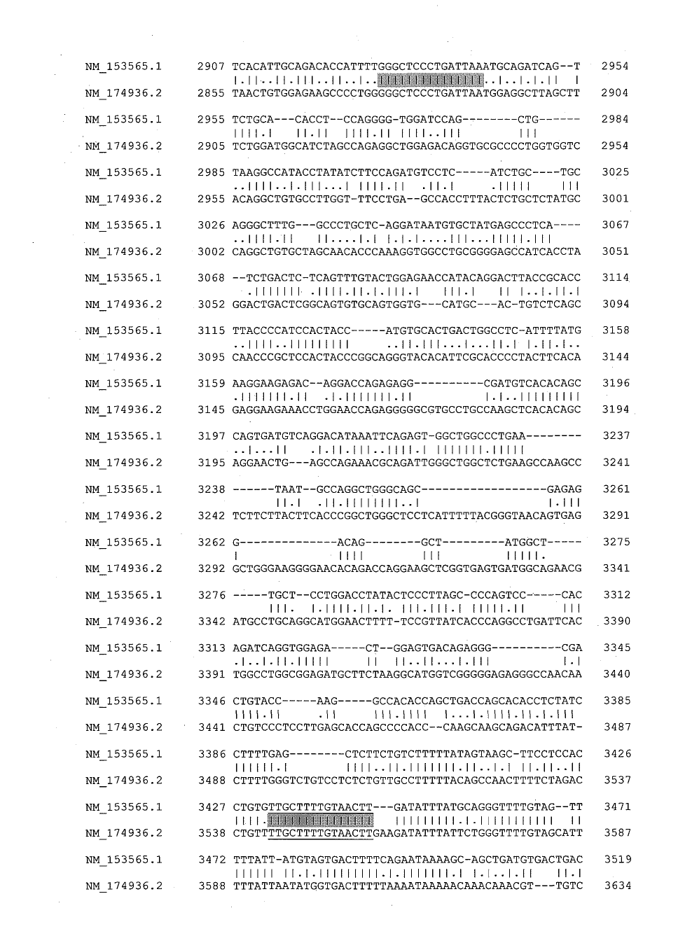

Suitable sub-sequences may be selected from a sequence which corresponds to a contiguous sequence present in one of the nucleic acid sequences selected from the group consisting of SEQ ID NO 14, SEQ ID NO 15, SEQ ID No 16, SEQ ID NO 17, SEQ ID NO 18 and SEQ ID NO 19, or a sequence selected from the group of (antisense) sequences shown in tables 2 and 3, and (the complement of) the sequences of the highlighted (shaded) sequences (of complementarity between human and mouse PCSK9 mRNA) shown in FIG. 17.

Preferred subsequences comprise or consist of at least 8, such as at least 10, such as at least 12, such as at least 13, such as at least 14 contiguous nucleobases which correspond to an equivalent nucleotide sequence present in any one of SEQ ID NO 3, SEQ ID NO 4, SEQ ID NO 5, SEQ ID NO 6, SEQ ID NO 7, or SEQ ID NO 8, most preferably SEQ ID NO 3 or SEQ ID NO 4.

The compound may further comprise a 5′ flanking nucleobase sequence, or a 3′ flanking sequence, or both a 5′ and a 3′ flanking sequence which is/are contiguous to said subsequence, wherein said flanking sequence or sequences consist of a total of between 2 and 22 nucleobase units, which when combined with said sub-sequence, the combined contiguous nucleobase sequence, i.e. consisting of said subsequence and said flanking sequence or sequences, is at least 80% homologous, such as at least 85% homologous, such as at least 90% homologous, such as at least 95% homologous, such as at least 97% homologous, such as 100% homologous to the corresponding sequence (such as the sense or preferably the antisense strand) present in the nucleic acid which encodes the PCSK9 polypeptide, such as SEQ ID NO 2, or a naturally occurring allelic variant thereof.

The flanking sequence or sequences may consist of a total of between 2 and 22 nucleobase units, such 3, 4, 5, 6, 7, 8, 9, 10, 11, 12, 13, 14, 15, 16, 17, 18, 19, 20, or 21 nucleobases, or such as between 4 to 12 nucleobases or such as between 2 and 10 nucleobases, such as between 5 to 10 nucleobases, or between 5 and 8 nucleobases, such as between 7 to 9 nucleobases.

In one embodiment said flanking sequence comprises of at least 2 nucleobase units which are 5′ to said sub-sequence.

In one embodiment said flanking sequence comprises between 1 and 6 nucleobase units which are 5′ to said sub-sequence.

In one embodiment said flanking sequence comprises of at least 2 nucleobase units which are 3′ to said sub-sequence.

In one embodiment said flanking sequence comprises between 1 and 6 nucleobase units which are 3′ to said sub-sequence

It is preferred that the sequences of each of the flanking sequences each form a contiguous sequence.

### The Combined Contiguous Nucleobase Sequence

The combined contiguous nucleobase sequence, i.e. consisting of said subsequence and, if present, said flanking sequence or sequences, is at least 80% homologous, such as at least 85% homologous, such as at least 90% homologous, such as at least 93% homologous, such as at least 95% homologous, such as at least 97% homologous, such as 100% homologous, to the corresponding sequence present in the nucleic acid which encodes the PCSK9 polypeptide, such as SEQ ID NO 2, or naturally occurring allelic variant thereof.

In one embodiment, the 3′ flanking sequence and/or 5′ flanking sequence may, independently, comprise or consist of between 1 and 10 nucleobases, such as 2, 3, 4, 5, 6, 7, 8, or 9 nucleobases, such as between 2 and 6 nucleobases, such as 3 or 4 nucleobases, which may be, in one embodiment nucleotide analogues, such as LNA units, or in another embodiment a combination of nucleotides and nucleotide analogues.

### Nucleobase Regions and Conjugates

It will be recognised that the compound of the invention which consists of a contiguous sequence of nucleobases (i.e. a nucleobase sequence), may comprise further non-nucleobase components, such as the conjugates herein referred to.

Therefore, in one embodiment, the compound of the invention may comprise both a polynucleotide region, i.e. a nucleobase region, and a further non-nucleobase region. When referring to the compound of the invention consisting of a nucleobase sequence, the compound may comprise non-nucleobase components, such as a conjugate component.

Alternatively, the compound of the invention may consist entirely of a (contiguous) nucleobase region.

In one embodiment the nucleobase portion and/or subsequence is selected from at least 9, least 10, least 11, least 12, least 13, least 14 and least 15 consecutive nucleotides or nucleotide analogues, which preferably are complementary to the target nucleic acid(s), although, as described above, may comprise one or two mismatches, with the corresponding sequence present in the nucleic acid which encodes the PCSK9 polypeptide, such as SEQ ID NO 2 or naturally occurring allelic variants thereof.

In one embodiment, the compound according to the invention consists of no more than 22 nucleobases, such as no more than 20 nucleobases, such as no more than 18 nucleobases, such as 15, 16 or 17 nucleobases, optionally conjugated with one or more non-nucleobase entity.

### RNA Antagonists

The nucleic acid which encodes a mammalian PCSK9 (target) may be in the sense or antisense orientation, preferably the sense orientation, such as the PCSK9 mRNA (of cDNA equivalent).

In one preferred embodiment, the compound may target a target nucleic acid which is an RNA transcript(s) of the gene(s) encoding the target proteins, such as mRNA or pre-mRNA, and may be in the form of a compound selected from the group consisting of; antisense inhibitors, antisense oligonucleotides, siRNA, miRNA, ribozymes and oligozymes.

It is highly preferable that the compound of the invention is an RNA antagonist, such as an antisense oligonucleotide or siRNA, preferably an antisense oligonucleotide.

Suitably, when the antisense oligonucleotide is introduced into the cell which is expressing the PCSK9 gene, results in reduction of the PCSK9 mRNA level, resulting in reduction in the level of expression of the PCSK9 in the cell.

The oligomers which target the PCSK9 mRNA, may hybridize to any site along the target mRNA nucleic acid, such as the 5′ untranslated leader, exons, introns and 3′ untranslated tail. However, it is preferred that the oligomers which target the PCSK9 mRNA hybridise to the mature mRNA form of the target nucleic acid.

When designed as an antisense inhibitor, for example, the oligonucleotides of the invention bind to the target nucleic acid and modulate the expression of its cognate protein. Preferably, such modulation produces an inhibition of expression of at least 10% or 20% compared to the normal expression level, more preferably at least a 30%, 40%, 50%, 60%, 70%, 80%, 90% or 95% inhibition compared to the normal expression level. Suitably, such modulation is seen when using between 5 and 25 nM concentrations of the compound of the invention. In the same of a different embodiment, the inhibition of expression is less than 100%, such as less than 98% inhibition, less than 95% inhibition, less than 90% inhibition, less than 80% inhibition, such as less than 70% inhibition. Modulation of expression level is determined by measuring protein levels, e.g. by the methods such as SDS-PAGE followed by western blotting using suitable antibodies raised against the target protein. Alternatively, modulation of expression levels can be determined by measuring levels of mRNA, eg. by northern blotting or quantitative RT-PCR. When measuring via mRNA levels, the level of down-regulation when using an appropriate dosage, such as between 5 and 25 nM concentrations, is, in one embodiment, typically to a level of between 10-20% the normal levels in the absence of the compound of the invention.

Preferably the compound according to the invention is an antisense oligonucleotide.

It is recognised that for the production of, for example, a siRNA, the compound of the invention may consist of a duplex of complementary sequence, i.e. a double stranded oligonucleotide, wherein each of the sequences in the duplex is as defined according to a compound of the invention. Typically, such siRNAs comprise of 2 complementary short RNA (or equivalent nucleobase) sequences, such as between 21 and 23 nts long, with, typically a 2 nt 3′ overhang on either end. In order to enhance in vivo update, the siRNAs may be conjugated, such as conjugated to a sterol, such as a cholesterol group (typically at the 3′ or 5′ termini of one or both of the strands). The siRNA may comprise nucleotide analogues such as LNA, as described in WO2005/073378 which is hereby incorporated by reference.

In one aspect of the invention the compound is not essentially double stranded, such as is not a siRNA.

In one embodiment, the compound of the invention does not comprise RNA (units).

The length of an oligomer (or contiguous nucleobase sequence) will be determined by that which will result in inhibition of the target. For a perfect match with the target, the contiguous nucleotide sequence or oligomer as low as 8 bases may suffice, but it will generally be more, e.g. 10 or 12, and preferably between 12-16. The maximum size of the oligomer will be determined by factors such as cost and convenience of production, ability to manipulate the oligomer and introduce it into a cell bearing the target mRNA, and also the desired binding affinity and target specificity. If too long, it may undesirably tolerate an increased number of mismatches, which may lead to unspecific binding.

The compound (oligomer or oligomeric compound) of the invention consists or comprises of between 10 and 50 nucleobases, such as between 10 and 30 nucleobases, such as 11, 12, 13, 14, 15, 16, 17, 18, 19, 20, 21, 22, 23, 24, 25, 26, 27, 28 or 29 nucleobases.

Particularly preferred compounds are antisense oligonucleotides comprising from about 10 to about 30 nucleobases, or from 12 to 25 nucleobases and in one embodiment are antisense compounds comprising 13-18 nucleobases such as 13, 14, 15, 16 or 17 nucleobases. In one embodiment, the oligomer according to the invention consists of no more than 22 nucleobases. In one embodiment it is preferred that the compound of the invention comprises less than 20 nucleobases.

In one embodiment, the oligomer according to the invention consists of no more than 22 nucleobases, such as no more than 20 nucleobases, such as no more than 18 nucleobases, such as 15, 16 or 17 nucleobases, optionally conjugated with one or more non-nucleobase entity, such as a conjugate.

In one embodiment, the oligomer or contiguous nucleobase sequence has a length of between 10-22 nucleobases.

In one embodiment, the oligomer or contiguous nucleobase sequence has a length of between 10-18 nucleobases.

In one embodiment, the oligomer or contiguous nucleobase sequence has a length of between 10-16 nucleobases.

In one embodiment, the oligomer or contiguous nucleobase sequence has a length of between 12-16 nucleobases.

In one embodiment, the oligomer or contiguous nucleobase sequence has a length of between 12-14 nucleobases.

In one embodiment, the oligomer or contiguous nucleobase sequence has a length of between 14-16 nucleobases.

In one embodiment, the oligomer or contiguous nucleobase sequence has a length of between 14-18 nucleobases.

In one embodiment, the oligomer or contiguous nucleobase sequence has a length of 14, 15 or 16 nucleobases.

In one embodiment, the oligomer or contiguous nucleobase sequence has a length of between 10-14 nucleobases, such as 10, 11, 12, 13 or 13 nucleobases. As disclosed in U.S. 60/977,409, such short oligonucleotides, i.e. “shortmers”, are surprisingly effecting at target down-regulation in vivo.

### Preferred Sequences

Target sequences of the invention may, in one non limiting embodiment, be identified as follows. In a first step conserved regions in the target gene are identified. Amongst those conserved regions, any sequences with polymorphisms are normally excluded (unless required for a specific purpose) as these may affect the binding specificity and/or affinity of an oligomer designed to bind to a target sequence in this region. Any regions with palindromic or repeat sequences are normally excluded. The remaining regions are then analysed and candidate target sequences of suitable length (such as the lengths of the oligomer/contiguous nucleobase sequence referred to herein), e.g. 10-50 nucleobases, preferably 10-25 nucleobases, more preferably 10, 11, 12, 13, 14, 15 or 16 nucleobases are identified. Target sequences which are, based on computer analysis, likely to form structures such as dimers or hairpin structures are normally excluded.

Preferably these candidate target sequences show a high degree of sequence homology throughout the animal kingdom—or at least among animals likely to be required for pre-clinical testing. This allows the use of the identified oligomer sequences, and the corresponding oligomers such as antisense oligonucleotides, to be tested in animal models. Particularly useful are target sequences which are conserved in human, chimpanzee, dog, rat, mouse, and most preferred in human, and mouse (and/or rat).

Suitable nucleobase sequences, such as motif sequences of the oligomers of the invention, are provided in Table 3, herein.

In one embodiment the contiguous nucleobase sequence is a contiguous nucleotide sequence present in a nucleic acid sequence shown in table 3, such as a contiguous nucleotide sequence selected from the group consisting of SEQ ID NO 40 to SEQ ID NO 393; SEQ ID 30 to SEQ ID 39; SEQ ID NOs 3, 4 and 5.

Other preferred oligonucleotides include sequences of 10, 11, 12, 13, 14, 15 and 16 continuous (such as contiguous) nucleobases selected from a sequence from the group consisting of SEQ ID NO 40 to SEQ ID NO 393; SEQ ID 30 to SEQ ID 39; SEQ ID NOs 3, 4 and 5.

Some preferred oligomers, and nucleobase sequences of the invention are shown in table 2.

In one embodiment the nucleobase portion (such as the contiguous nucleobase sequence) is selected from, or comprises, one of the following sequences: SEQ ID No 14, SEQ ID No 15, SEQ ID No 16, SEQ ID No 17, SEQ ID No 18 and SEQ ID No 19 or, in one embodiment a sub.sequence thereof, such as a sub.sequence of 10, 11, 12, 13, 14, 15 and 16 continuous (such as contiguous) nucleobases.

In one embodiment the contiguous nucleobase sequence is a contiguous nucleotide sequence present in a nucleic acid sequence selected from the group consisting of: SEQ ID NO 3, SEQ ID NO 4, SEQ ID NO 5, SEQ ID NO 6, SEQ ID NO 7, SEQ ID NO 8, SEQ ID 30, SEQ ID NO 31, SEQ ID NO 32, SEQ ID NO 33, SEQ ID NO 34, SEQ ID NO 35, SEQ ID NO 36, SEQ ID NO 37, SEQ ID NO 38, and SEQ ID NO 39, or, in one embodiment a sub.sequence thereof, such as a sub.sequence of 10, 11, 12, 13, 14, 15 and 16 continuous (such as contiguous) nucleobases.

In one embodiment the contiguous nucleobase or oligomer is selected from the group consisting of: SEQ ID NO 10, SEQ ID NO 20, SEQ ID NO 11, SEQ ID NO 9, SEQ ID NO 21, SEQ ID NO 22, SEQ ID NO 23, SEQ ID NO 24, SEQ ID NO 25, SEQ ID NO 26, SEQ ID NO 27, SEQ ID NO 28, and SEQ ID NO 29 or, in one embodiment a sub.sequence thereof, such as a sub.sequence of 10, 11, 12, 13, 14, 15 and 16 continuous (such as contiguous) nucleobases.

In one embodiment the nucleobase portion is selected from, or comprises, one of the following sequences: SEQ ID NO 3, SEQ ID NO 4, SEQ ID NO 5, SEQ ID NO 6, SEQ ID NO 7, and SEQ ID NO 8, (preferably SEQ ID NO 3 and SEQ ID NO 4) or, in one embodiment a sub.sequence thereof, such as a sub.sequence of 10, 11, 12, 13, 14, 15 and 16 continuous (such as contiguous) nucleobases.

Other preferred oligonucleotides include sequences of 10, 11, 12, 13, 14, 15 and 16 continuous (such as contiguous) nucleobases selected from a sequence from the group consisting of SEQ ID NO 9, 10 and 11. Further preferred aspect of the invention is directed to compounds consisting or comprising of SEQ ID NO 9, 10 or 11.

It will be understood by the skilled person, that in one embodiment when referring to specific gapmer oligonucleotide sequences, such as those provided herein (e.g. SEQ ID NOS 9, 10 and 11) when the linkages are phosphorothioate linkages, alternative linkages, such as those disclosed herein may be used, for example phosphate linkages may be used, particularly for linkages between nucleotide analogues, such as LNA, units. Likewise, when referring to specific gapmer oligonucleotide sequences, such as those provided herein (e.g. SEQ ID NOS 9, 10 and 11), when the C residues are annotated as 5′ methyl modified cytosine, in one embodiment, one or more of the Cs present in the oligonucleotide may be unmodified C residues.

In one embodiment the nucleobase sequence consists or comprises of a sequence which is, or corresponds to, a sequence selected from the group consisting of: SEQ ID NO 3, SEQ ID NO 4, SEQ ID NO 5, SEQ ID NO 6, SEQ ID NO 7 and SEQ ID NO 8, or a contiguous sequence of at least 12, 13, 14, 15, or 16 consecutive nucleobases present in said sequence, wherein the nucleotides present in the compound may be substituted with a corresponding nucleotide analogue and wherein said compound may comprise one, two, or three mismatches against said selected sequence.

In one embodiment the compound according to the invention consists or comprises of SEQ ID NO 3 or an equivalent nucleobase sequence.

In one embodiment the compound according to the invention consists or comprises of SEQ ID NO 4 or an equivalent nucleobase sequence.

In one embodiment the compound according to the invention consists or comprises of SEQ ID NO 5 or an equivalent nucleobase sequence.

In one embodiment the compound according to the invention consists or comprises of SEQ ID NO 6 or an equivalent nucleobase sequence.

In one embodiment the compound according to the invention consists or comprises of SEQ ID NO 7 or an equivalent nucleobase sequence.

In one embodiment the compound according to the invention consists or comprises of SEQ ID NO 8 or an equivalent nucleobase sequence.

In one embodiment the compound according to the invention consists or comprises of SEQ ID NO 9.

In one embodiment the compound according to the invention consists or comprises of SEQ ID NO 10.

In one embodiment the compound according to the invention consists or comprises of SEQ ID NO 11.

In one embodiment the compound according to the invention consists or comprises of SEQ ID NO 30, SEQ ID NO 31, SEQ ID NO 32, SEQ ID NO 33, SEQ ID NO 34, SEQ ID NO 35, SEQ ID NO 36, SEQ ID NO 37, SEQ ID NO 38, or SEQ ID NO 39.

Other oligomers of the invention include sequences of 10, 11, 12, 13, 14, 15 and 16 continuous (such as contiguous) nucleobases selected from one of the above listed SEQ IDs or the compound IDs# as referred to in the examples.

Other oligomers of the invention include sequences of 10, 11, 12, 13, 14, 15 and 16 continuous (such as contiguous) nucleobases selected from a sequence from the group consisting of SEQ ID No 14, SEQ ID No 15, SEQ ID No 16, SEQ ID No 17, SEQ ID No 18 and SEQ ID No 19, or a sequence selected from the group of (antisense) sequences shown in table 2 or table 3.

Other oligomers of the invention include sequences of 10, 11, 12, 13, 14, 15 and 16 continuous (such as contiguous) nucleobases selected from a sequence from the group consisting of SEQ ID NO 3, SEQ ID NO 4, SEQ ID NO 5, SEQ ID NO 6, SEQ ID NO 7, SEQ ID NO 8, SEQ ID NO 47, SEQ ID NO 49, SEQ ID NO 54, SEQ ID NO 56, SEQ ID NO 118, SEQ ID NO 136, and SEQ ID NO 139.

Preferred compounds consist of 10, 11, 12, 13, 14, 15 or 16 continuous (such as contiguous) nucleobases which correspond to a nucleotide sequence present in a sequence selected from the group consisting of SEQ ID NO 3, SEQ ID NO 4, SEQ ID NO 5, SEQ ID NO 6, SEQ ID NO 7, and SEQ ID NO 8, or preferably SEQ ID NO 3 and SEQ ID NO 4.

Further preferred compounds are shown in Tables 2 and table 3. of U.S. 60/828,735 and table 4 of U.S. 60/972,932, which are hereby specifically incorporated into this specification (as referred to in the specific list of embodiments listed herein).

Suitably, the oligomer according to the invention consists or comprises one of the above mentioned SEQ ID sequences.

### Complementarity and Mismatches

In one embodiment, the compound of the invention consists of a (contiguous) nucleobase sequence with is 100% complementary to a corresponding (contiguous) region of the corresponding sequence present in the nucleic acid which encodes the PCSK9 polypeptide, such as SEQ ID NO 2 or naturally occurring allelic variants thereof.

However, in one embodiment, the compound of the invention preferably does not comprise more than four, such as not more than three, such as not more than two, such as not more than one mismatch, with the corresponding region of the sequence present in the nucleic acid which encodes the PCSK9 polypeptide, such as SEQ ID NO 2 or naturally occurring allelic variants thereof.

When the subsequence consists of 8 or 9 nucleobases, it may preferably comprise at most only one mismatch with the corresponding region of SEQ ID NO 2 or naturally occurring allelic variants thereof, such as no mismatch. However, for longer subsequences of at least 10, such as at least 11 nucleobases, such as at least 12, at least 13, at least 14 or at least 15 nucleobases, additional mismatches may be introduced, such as a total of one, two, three or four mismatches with the corresponding region of SEQ ID NO 2 or naturally occurring allelic variants thereof, may be introduced into the subsequence. However, in regards to longer subsequences of at least 10 nucleobases, as listed above, the subsequence may comprise at least a core contiguous sequence of at least 8 nucleobases, wherein within the core contiguous sequence, at most, only one mismatch with the corresponding region of the sequence present in the nucleic acid which encodes the PCSK9 polypeptide, such as SEQ ID NO 2, or naturally occurring allelic variants thereof is allowed and preferably no mismatches.

In one embodiment, the compound is at least 80%, such as at least 85%, such as at least 90%, such as at least 91%, such as at least 92%, such as at least 93%, such as at least 93⅓%, such as at least 93.75%, such as at least 94%, such as at least 95%, 9 such as at least 6% or at least 97% complementary, such as 100% complementary to the corresponding region of the nucleic acid target sequence, such as the mRNA which encodes the PCSK9 polypeptide, such as SEQ ID NO 2.

Referring to the principles by which the compound, can elicit its therapeutic action, the target of the present invention may be the mRNA derived from the corresponding sequence present in the nucleic acid which encodes the PCSK9 polypeptide, such as SEQ ID NO 2 or naturally occurring allelic variants thereof.

It will be recognised that when referring to a preferred nucleotide sequence motif or nucleotide sequence, which consists of only nucleotides, the compounds of the invention which are defined by that sequence may comprise a corresponding nucleotide analogues in place of one or more of the nucleotides present in said sequence, such as LNA units or other nucleotide analogues which raise the Tₘ of the oligonucleotide/target duplex—such as the nucleotide analogues described below, particularly LNA and/or 2′ substituted nucleotides (2′ modified).

### Nucleotide Analogues

In one embodiment, at least one of the nucleobases present in the oligomers a modified nucleobase selected from the group consisting of 5-methylcytosine, isocytosine, pseudoisocytosine, 5-bromouracil, 5-propynyluracil, 6-aminopurine, 2-aminopurine, inosine, diaminopurine, and 2-chloro-6-aminopurine.

It will be recognised that when referring to a preferred nucleotide sequence motif or nucleotide sequence, which consists of only nucleotides, the oligomers of the invention which are defined by that sequence may comprise a corresponding nucleotide analogues in place of one or more of the nucleotides present in said sequence, such as LNA units or other nucleotide analogues, which raise the duplex stability/Tₘ of the oligomer/target duplex (i.e. affinity enhancing nucleotide analogues).

Furthermore, the nucleotide analogues may enhance the stability of the oligomer in vivo.

Incorporation of affinity-enhancing nucleotide analogues in the oligomer nucleobase sequence, such as LNA or 2′-substituted sugars, preferably LNA, can allow the size of the specifically binding oligonucleotide to be reduced, and may also reduce the upper limit to the size of the oligonucleotide before non-specific or aberrant binding takes place. An affinity enhancing nucleotide analogue is one which, when inserted into the nucleobase sequence of the oligomer results in a increased Tₘ of the oligomer when formed in a duplex with a complementary RNA (such as the mRNA target), as compared to an equivalent oligomer which comprises a DNA nucleotide in place of the affinity enhancing nucleotide analogue

Examples of suitable and preferred nucleotide analogues are provided by PCT/DK2006/000512 or are referenced therein.

In some embodiments at least one of said nucleotide analogues is 2′-MOE-RNA, such as 2, 3, 4, 5, 6, 7 or 8 2′-MOE-RNA nucleobase units.

In some embodiments at least one of said nucleotide analogues is 2′-fluoro DNA, such as 2, 3, 4, 5, 6, 7 or 8 2′-fluoro-DNA nucleobase units.

Specific examples of nucleoside analogues which may be utilised in the oligomers of the present invention are described by e.g. Freier & Altmann; Nucl. Acid Res., 1997, 25, 4429-4443 and Uhlmann; Curr. Opinion in Drug Development, 2000, 3(2), 293-213, and in Scheme 1:

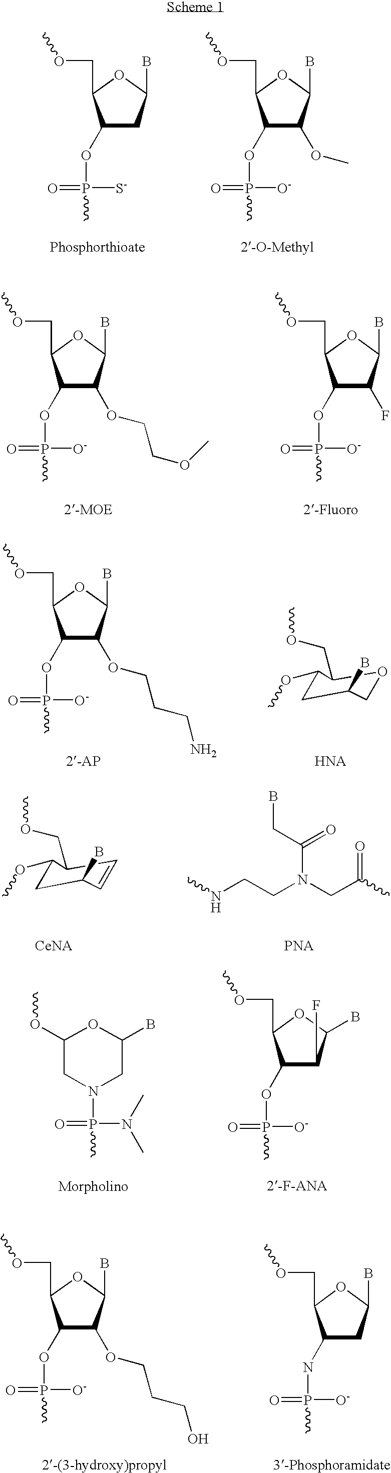

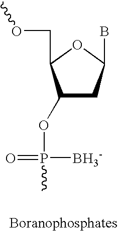

The term “LNA” refers to a bicyclic nucleotide analogue, known as “Locked Nucleic Acid”. It may refer to an LNA monomer, or, when used in the context of an “LNA oligonucleotide” refers to an oligonucleotide containing one or more such bicyclic nucleotide analogues. The LNA used in the oligonucleotide compounds of the invention preferably has the structure of the general formula

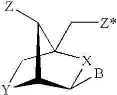

where X and Y are independently selected among the groups —O—,
—S—, —N(H)—, N(R)—, —CH₂— or —CH— (if part of a double bond),
—CH₂—O—, —CH₂—S—, —CH₂—N(H)—, —CH₂—N(R)—, —CH₂—CH₂— or —CH₂—CH— (if part of a double bond), —CH═CH—, where R is selected from hydrogen and C₁₋₄-alkyl; Z and Z* are independently selected among an internucleoside linkage, a terminal group or a protecting group; B constitutes a natural or non-natural nucleotide base moiety; and the asymmetric groups may be found in either orientation.

Preferably, the LNA used in the oligomer of the invention comprises at least one LNA unit according any of the formulas

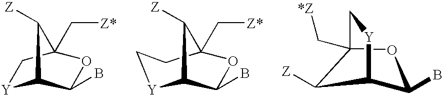

wherein Y is —O—, —S—, —NH—, or N(R^{H}); Z and Z* are independently selected among an internucleoside linkage, a terminal group or a protecting group; B constitutes a natural or non-natural nucleotide base moiety, and R^{H} is selected from hydrogen and C₁₋₄-alkyl.

Preferably, the Locked Nucleic Acid (LNA) used in the oligomeric compound, such as an antisense oligonucleotide, of the invention comprises at least one nucleotide comprises a Locked Nucleic Acid (LNA) unit according any of the formulas shown in Scheme 2 of PCT/DK2006/000512.

Preferably, the LNA used in the oligomer of the invention comprises internucleoside linkages selected from —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —S—P(O)₂—S—, —O—PO(R^{H})—O—, O—PO(OCH₃)—O—, —O—PO(NR^{H})—O—, —O—PO(OCH₂CH₂S—R)—O—, —O—PO(BH₃)—O—, —O—PO(NHR^{H})—O—, —O—P(O)₂—NR^{H}—, —NR^{H}—P(O)₂—O—, —NR^{H}—CO—O—, where R^{H} is selected form hydrogen and C₁₋₄-alkyl.

Specifically preferred LNA units are shown in scheme 2:

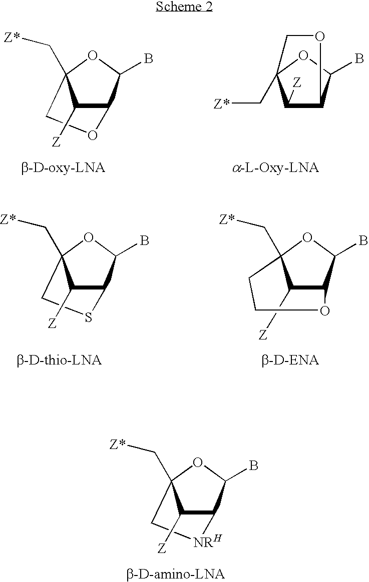

The term “thio-LNA” comprises a locked nucleotide in which at least one of X or Y in the general formula above is selected from S or —CH₂—S—. Thio-LNA can be in both beta-D and alpha-L-configuration.

The term “amino-LNA” comprises a locked nucleotide in which at least one of X or Y in the general formula above is selected from —N(H)—, N(R)—, CH₂—N(H)—, and —CH₂—N(R)— where R is selected from hydrogen and C₁₋₄-alkyl. Amino-LNA can be in both beta-D and alpha-L-configuration.

The term “oxy-LNA” comprises a locked nucleotide in which at least one of X or Y in the general formula above represents —O— or —CH₂—O—. Oxy-LNA can be in both beta-D and alpha-L-configuration.

The term “ena-LNA” comprises a locked nucleotide in which Y in the general formula above is —CH₂—O— (where the oxygen atom of —CH₂—O— is attached to the 2′-position relative to the base B).

In a preferred embodiment LNA is selected from beta-D-oxy-LNA, alpha-L-oxy-LNA, beta-D-amino-LNA and beta-D-thio-LNA, in particular beta-D-oxy-LNA.

Preferably, within the compound according to the invention, such as an antisense oligonucleotide, which comprises LNA, all LNA C residues are 5′ methyl-Cytosine.

Preferably the LNA units of the compound, such as an antisense oligonucleotide, of the invention are selected from one or more of the following: thio-LNA, amino-LNA, oxy-LNA, ena-LNA and/or alpha-LNA in either the D-beta or L-alpha configurations or combinations thereof. Beta-D-oxy-LNA is a preferred LNA for use in the oligomeric compounds of the invention. Thio-LNA may also be preferred for use in the oligomeric compounds of the invention. Amino-LNA may also be preferred for use in the oligomeric compounds of the invention. Oxy-LNA may also be preferred for use in the oligomeric compounds of the invention. Ena-LNA may also be preferred for use in the oligomeric compounds of the invention. Alpha-LNA may also be preferred for use in the oligomeric compounds of the invention.

The Locked Nucleic Acid (LNA) used in the compound, such as an antisense oligonucleotide, of the invention has the structure of the general formula shown in scheme 1 of PCT/DK2006/000512. The terms “thio-LNA”, “amino-LNA”, “oxy-LNA”, “ena-LNA”, “alpha-L-LNA”, “LNA derivatives”, “locked nucleotide” and “locked nucleobase” are also used as defined in PCT/DK2006/000512.

Suitably, when the nucleobase sequence of the oligomer, or the contiguous nucleobase sequence, is not fully complementary to the corresponding region of the PCSK9 target sequence, in one embodiment, when the oligomer comprises affinity enhancing nucleotide analogues, such nucleotide analogues form a complement with their corresponding nucleotide in the PCSK9 target.

The oligomer may thus comprise or consist of a simple sequence of natural nucleotides—preferably 2′-deoxynucleotides (referred to here generally as “DNA”), but also possibly ribonucleotides (referred to here generally as “RNA”)—or it could comprise one or more (and possibly consist completely of) nucleotide “analogues”.

Nucleotide “analogues” are variants of natural DNA or RNA nucleotides by virtue of modifications in the sugar and/or base and/or phosphate portions. The term “nucleobase” will be used to encompass natural (DNA- or RNA-type) nucleotides as well as such “analogues” thereof. Analogues could in principle be merely “silent” or “equivalent” to the natural nucleotides in the context of the oligonucleotide, i.e. have no functional effect on the way the oligonucleotide works to PCSK9 expression. Such “equivalent” analogues may nevertheless be useful if, for example, they are easier or cheaper to manufacture, or are more stable to storage or manufacturing conditions, or represent a tag or label. Preferably, however, the analogues will have a functional effect on the way in which the oligomer works to inhibit expression; for example by producing increased binding affinity to the target and/or increased resistance to intracellular nucleases and/or increased ease of transport into the cell.

Examples of such modification of the nucleotide include modifying the sugar moiety to provide a 2′-substituent group or to produce a bridged (locked nucleic acid) structure which enhances binding affinity and probably also provides some increased nuclease resistance; modifying the internucleotide linkage from its normal phosphodiester to one that is more resistant to nuclease attack, such as phosphorothioate or boranophosphate—these two, being cleavable by RNase H, also allow that route of antisense inhibition in modulating the PCSK9 expression.

In some embodiments, the oligomer comprises from 3-8 nucleotide analogues, e.g. 6 or 7 nucleotide analogues. In the by far most preferred embodiments, at least one of said nucleotide analogues is a locked nucleic acid (LNA); for example at least 3 or at least 4, or at least 5, or at least 6, or at least 7, or 8, of the nucleotide analogues may be LNA. In some embodiments all the nucleotides analogues may be LNA.

In some embodiments the nucleotide analogues present within the oligomer of the invention in regions A and C mentioned herein are independently selected from, for example: 2′-O-alkyl-RNA units, 2′-amino-DNA units, 2′-fluoro-DNA units, LNA units, arabino nucleic acid (ANA) units, 2′-fluoro-ANA units, HNA units, INA (intercalating nucleic acid) units and 2′MOE units. It is also considered that the nucleotide analogues present in an oligomer of the invention are all the same, all be it, allowing for base variation.

2′-O-methoxyethyl-RNA (2′MOE), 2′-fluoro-DNA monomers and LNA are preferred nucleotide analogues, and as such the oligonucleotide of the invention may comprise nucleotide analogues which are independently selected from these three types of analogue, or may comprise only one type of analogue selected from the three types.

Compounds according to the invention, are, in one embodiment, those consisting or comprising a sequence selected from SEQ ID NO 3, SEQ ID NO 4, SEQ ID NO 5, SEQ ID NO 6, SEQ ID NO 7, and SEQ ID NO 8, or preferably SEQ ID NO 3 or SEQ ID NO 4. wherein, in one embodiment the nucleotides present in the compound may be substituted with a corresponding nucleotide analogue and, wherein said compound may comprise one, two or three mismatches against said selected sequence.

Preferred compounds according to the invention are those consisting or comprising of SEQ ID NOS 3 or 4, wherein they contain at least one nucleic acid analogue, wherein in one embodiment, the LNA units may be substituted with an alternative corresponding nucleotide analogue, and wherein said compound may comprise one, two, or three mismatches against said selected sequence.

Nucleotide analogues which increase the Tₘ of the oligonucleotide/target nucleic acid target, as compared to the equivalent nucleotide are preferred.

Preferably, the compound according to the invention comprises at least one nucleotide analogue, such as Locked Nucleic Acid (LNA) unit, such as 4, 5, 6, 7, 8, 9, or 10 nucleotide analogues, such as Locked Nucleic Acid (LNA) units, preferably between 4 to 9 nucleotide analogues, such as LNA units, such as 6-9 nucleotide analogues, such as LNA units, most preferably 6, 7 or 8 nucleotide analogues, such as LNA units.

The term LNA is used as defined in PCT application PCT/DK2006/000512, which is hereby incorporated by reference.

Preferably the LNA units comprise at least one beta-D-oxy-LNA unit(s) such as 2, 3, 4, 5, 6, 7, 8, 9, or 10 beta-D-oxy-LNA units. The compound of the invention, such as the antisense oligonucleotide, may comprise more than one type of LNA unit. Suitably, the compound may comprise both beta-D-oxy-LNA, and one or more of the following LNA units: thio-LNA, amino-LNA, oxy-LNA, ena-LNA and/or alpha-LNA in either the D-beta or L-alpha configurations or combinations thereof.

Preferably, the compound, such as an antisense oligonucleotide, may comprise both nucleotide analogues, such as LNA units, and DNA units. Preferably the combined total of nucleobases, such as, LNA and DNA units, is between 10-20, such as 14-20, such as between 15-18, such as 15, 16 or 17 nucleobase units, or is a shortmer as referred to herein. Preferably the ratio of nucleotide analogues to DNA present in the oligomeric compound of the invention is between 0.3 and 1, more preferably between 0.4 and 0.9, such as between 0.5 and 0.8.

Preferably, the compound of the invention, such as an antisense oligonucleotide, consists of a total of 10-25, or 12-25 nucleotides and/or nucleotide analogues, wherein said compound comprises a subsequence of at least 8 nucleotides or nucleotide analogues, said subsequence being located within (i.e. corresponding to) a sequence selected from the group consisting of SEQ ID No 14, SEQ ID No 15, SEQ ID No 16, SEQ ID No 17, SEQ ID No 18 and SEQ ID No 19.

In one aspect of the invention, the nucleotides (and/or nucleotide analogues) are linked to each other by means of a phosphorothioate group. An interesting embodiment of the invention is directed to compounds selected from the group consisting of SEQ ID NO 3, SEQ ID NO 4, SEQ ID NO 5, SEQ ID NO 6, SEQ ID NO 7, and SEQ ID NO 8, wherein each linkage group within each compound is a phosphorothioate group. Such modifications are denoted by the subscript S.

The tables referred to herein provide further nucleobase sequences of compounds of the invention.

In further embodiments, the compound of the invention, such as the antisense oligonucleotide of the invention may comprises or consist of 13, 14, 15, 16, 17, 18, 19, 20 or 21 nucleobases.

Preferably the compound according to the invention, such as an antisense oligonucleotide, comprises or consists of 2, 3, 4, 5, 6, 7, 8, 9, 10, 11, 12, 13, 14 or 15 nucleotide analogues, such as LNA units, in particular 4, 5, 6, 7, 8, 9 or 10 nucleotide analogues, such as LNA units, such as between 1 and 10 nucleotide analogues, such as LNA units such as between 2 and 8 nucleotide analogues such as LNA units.

### RNAseH Recruitment

It is preferable that said subsequence or combined nucleobase sequence comprises a continuous (contiguous) sequence of at least 7 nucleobase residues, such as at least 8 or at least 9 nucleobase residues, including 7, 8 or 9 nucleobases, which, when formed in a duplex with the complementary target RNA corresponding to each of said polynucleotides which encode said mammalian PCSK9 are capable of recruiting RNaseH, such as DNA nucleotides.

The size of the contiguous sequence which is capable of recruiting RNAseH may be higher, such as 10, 11, 12, 13, 14, 15, 16, 17, 18, 19 or 20 nucleobase units.

The contiguous sequence which is capable of recruiting RNAseH may be region B as referred to in the context of a gapmer as described herein.

EP 1 222 309 provides in vitro methods for determining RNaseH activity, which may be used to determine the ability to recruit RNaseH. A compound is deemed capable of recruiting RNase H if, when provided with the complementary RNA target, it has an initial rate, as measured in pmol/l/min, of at least 1%, such as at least 5%, such as at least 10% or less than 20% of the equivalent DNA only oligonucleotide, with no 2′ substitutions, with phosphorothioate linkage groups between all nucleotides in the oligonucleotide, using the methodology provided by Example 91-95 of EP 1 222 309.

A compound is deemed essentially incapable of recruiting RNaseH if, when provided with the complementary RNA target, and RNaseH, the RNaseH initial rate, as measured in pmol/l/min, is less than 1%, such as less than 5%, such as less than 10% or less than 20% of the initial rate determined using the equivalent DNA only oligonucleotide, with no 2′ substitutions, with phosphiothioate linkage groups between all nucleotides in the oligonucleotide, using the methodology provided by Example 91-95 of EP 1 222 309.

However, it is also recognised that antisense oligonucleotides may function via non RNaseH mediated degradation of target mRNA, such as by steric hindrance of translation, or other methods.

The compound of the invention may comprise a nucleobase sequence which comprises both nucleotides and nucleotide analogues, and may be in the form of a gapmer, a headmer or a mixmer.

A headmer is defined by a contiguous stretch of nucleotide analogues at the 5′-end followed by a contiguous stretch of DNA or modified nucleobases units recognizable and cleavable by the RNaseH towards the 3′-end (such as at least 7 such nucleobases), and a tailmer is defined by a contiguous stretch of DNA or modified monomers recognizable and cleavable by the RNaseH at the 5′-end (such as at least 7 such nucleobases), followed by a contiguous stretch of nucleotide analogues towards the 3′-end. Other chimeras according to the invention, called mixmers consisting of an alternate composition of DNA or modified monomers recognizable and cleavable by RNaseH and nucleotide analogues. Some nucleotide analogues may also be able to mediate RNaseH binding and cleavage. Since α-L-LNA recruits RNaseH activity to a certain extent, smaller gaps of DNA or modified monomers recognizable and cleavable by the RNaseH for the gapmer construct might be required, and more flexibility in the mixmer construction might be introduced.

### Gapmers

Preferably, the compound of the invention is an antisense oligonucleotide which is a gapmer.

Preferably the gapmer comprises a (poly)nucleobase sequence of formula (5′ to 3′), A-B-C (and optionally D), wherein; A (5′ region) consists or comprises of at least one nucleotide analogue, such as at least one LNA unit, such as between 1-6 nucleotide analogues, such as LNA units, preferably between 2-5 nucleotide analogues, such as 2-5 LNA units, such as 3 or 4 nucleotide analogues, such as 3 or 4 LNA units and; B (central domain), preferably immediately 3′ (i.e. contiguous) to A, consists or comprises at least one DNA sugar unit, such as 1-12 DNA units, preferably between 4-12 DNA units, more preferably between 6-10 DNA units, such as between 7-10 DNA units, most preferably 8, 9 or 10 DNA units, and; C (3′ region) preferably immediately 3′ to B, consists or comprises at of at least one nucleotide analogues, such as at least one LNA unit, such as between 1-6 nucleotide analogues, such between 2-5 nucleotide analogues, such as between 2-5 LNA units, most preferably 3 or 4 nucleotide analogues, such as 3 or 4 LNA units. Preferred gapmer designs are disclosed in WO2004/046160.

Preferred gapmer designs include, when:
A Consists of 3 or 4 consecutive nucleotide analogues
B Consists of 7 to 10 consecutive DNA nucleotides or equivalent nucleobases which are capable of recruiting RNAseH
C Consists of 3 or 4 consecutive nucleotide analogues
D Consists, where present, of one DNA nucleotide.
Or when
A Consists of 3 consecutive nucleotide analogues
B Consists of 9 consecutive DNA nucleotides or equivalent nucleobases which are capable of recruiting RNAseH
C Consists of 3 consecutive nucleotide analogues
D Consists, where present, of one DNA nucleotide.
Or when
A Consists of 4 consecutive nucleotide analogues
B Consists of 8 consecutive DNA nucleotides or equivalent nucleobases which are capable of recruiting RNAseH
C Consists of 4 consecutive nucleotide analogues
D Consists, where present, of one DNA nucleotide.
Or when
A Consists of 2 consecutive nucleotide analogues
B Consists of 8 consecutive DNA nucleotides or equivalent nucleobases which are capable of recruiting RNAseH
C Consists of 3 consecutive nucleotide analogues
D Consists, where present, of one DNA nucleotide.
Or when
A Consists of 3 consecutive nucleotide analogues
B Consists of 8 consecutive DNA nucleotides or equivalent nucleobases which are capable of recruiting RNAseH
C Consists of 2 consecutive nucleotide analogues
D Consists, where present, of one DNA nucleotide.
Or when
A Consists of 2 consecutive nucleotide analogues
B Consists of 8 consecutive DNA nucleotides or equivalent nucleobases which are capable of recruiting RNAseH
C Consists of 2 consecutive nucleotide analogues
D Consists, where present, of one DNA nucleotide.

The DNA nucleotides in the central domain (B) may be substituted with one or more, or even all the DNA nucleotides may be substituted with a nucleobase, including nucleotide analogues which are capable or recruiting RNAse H.

In the above embodiments referring to gapmer designs, the gap region ‘B’ may alternatively be 7, 8, 9 or 10 consecutive DNA nucleotides or equivalent nucleobases which are capable of recruiting RNAseH.

In a gapmer oligonucleotide, it is highly preferable that any mismatches are not within the central domain (B) above, are at least within a minimum stretch of 7 continuous nucleobases of the central domain, such as 7, 8 or 9 or 10 continuous nucleobases, which preferably comprises or consists of DNA units.

In a gapmer oligonucleotide, it is preferred that any mismatches are located towards the 5′ or 3′ termini of the gapmer. Therefore, it is preferred that in a gapmer oligonucleotide which comprises mismatches with the target mRNA, that such mismatches are located either in 5′ (A) and/or 3′ (C) regions, and/or said mismatches are between the 5′ or 3′ nucleotide unit of said gapmer oligonucleotide and target molecule.

Preferably, the gapmer, of formula A-B-C, further comprises a further region, D, which consists or comprises, preferably consists, of one or more DNA sugar residue terminal of the 3′ region (C) of the oligomeric compound, such as between one and three DNA sugar residues, including between 1 and 2 DNA sugar residues, most preferably 1 DNA sugar residue.

### Shortmers

US provisional application, 60/977,409, hereby incorporated by reference, refers to ‘shortmer’ oligonucleotides, which, in one embodiment particularly, are preferred oligomeric compounds according to the present invention

In one embodiment oligomer consisting of a contiguous nucleobase sequence of a total of 10, 11, 12, 13 or 14 nucleobase units, wherein the contiguous nucleobase sequence is of formula (5′-3′), A-B-C, or optionally A-B-C-D. wherein: A consists of 1, 2 or 3 LNA units; B consists of 7, 8 or 9 contiguous nucleobase units which are capable of recruiting RNAseH when formed in a duplex with a complementary RNA molecule (such as a mRNA target); and C consists of 1, 2 or 3 LNA units. When present, D consists of a single DNA unit. In one embodiment, there is no region D. In one embodiment A consists of 1 LNA unit. In one embodiment A consists of 2 LNA units. In one embodiment A consists of 3 LNA units. In one embodiment C consists of 1 LNA unit. In one embodiment C consists of 2 LNA units. In one embodiment C consists of 3 LNA units. In one embodiment B consists of 7 nucleobase units. In one embodiment B consists of 8 nucleobase units. In one embodiment B consists of 9 nucleobase units. In one embodiment B comprises of between 1-9 DNA units, such as 2, 3, 4, 5, 6, 7 or 8 DNA units. In one embodiment B consists of DNA units. In one embodiment B comprises of at least one LNA unit which is in the alpha-L configuration, such as 2, 3, 4, 5, 6, 7, 8 or 9 LNA units in the alpha-L-configuration. In one embodiment B comprises of at least one alpha-L-oxy LNA unit or wherein all the LNA units in the alpha-L-configuration are alpha-L-oxy LNA units. In one embodiment the number of nucleobases in A-B-C are selected from the group consisting of: 1-8-2, 2-8-1, 2-8-2, 3-8-3, 2-8-3, 3-8-2. In one embodiment the number of nucleobases in A-B-C are selected from the group consisting of: 1-9-1, 1-9-2, 2-9-1, 2-9-2, 3-9-2, and 2-9-3. In one embodiment the number of nucleobases in A-B-C are selected from the group consisting of: 2-7-1, 1-7-2, 2-7-2, 3-7-3, 2-7-3, 3-7-2, 3-7-4, and 4-7-3. In one embodiment both A and C both consist of two LNA units each, and B consists of 8 nucleobase units, preferably DNA units. In one embodiment the LNA units of A and C are independently selected from oxy-LNA, thio-LNA, and amino-LNA, in either of the beta-D and alpha-L configurations or combinations thereof. In one embodiment the LNA units of A and C are beta-D-oxy-LNA. In one embodiment the internucleoside linkages are independently selected from the group consisting of: phosphodiester, phosphorothioate and boranophosphate. In one embodiment the oligomer comprises at least one phosphorothioate internucleoside linkage. In one embodiment the internucleoside linkages adjacent to or between DNA units are phosphorothioate linkages. In one embodiment the linkages between at least one pair of consecutive LNA units, such as 2 LNA units in region A or C, is a phosphodiester linkage. In one embodiment the linkages between consecutive LNA units such as 2 LNA units in region A and C, are phosphodiester linkages. In one embodiment the all the internucleoside linkages are phosphorothioate linkages.

Suitable internucleoside linkages include those listed within PCT/DK2006/000512, for example the internucleoside linkages listed on the first paragraph of page 34 of PCT/DK2006/000512.

Suitable sulphur (S) containing internucleoside linkages as provided above may be preferred. Phosphorothioate internucleotide linkages are also preferred, particularly for the gap region (B) of gapmers. Phosphorothioate linkages may also be used for the flanking regions (A and C, and for linking C to D, and D).

Regions A, B and C, may however comprise internucleoside linkages other than phosphorothioate, such as phosphodiester linkages, particularly, for instance when the use of nucleotide analogues protects the internucleoside linkages within regions A and C from endo-nuclease degradation—such as when regions A and C comprise LNA nucleobases.

The internucleobase linkages in the oligomer may be phosphodiester, phosphorothioate or boranophosphate so as to allow RNase H cleavage of targeted RNA. Phosphorothioate is preferred, for improved nuclease resistance and other reasons, such as ease of manufacture.

In one aspect of the oligomer of the invention, the nucleobases (nucleotides and/or nucleotide analogues) are linked to each other by means of phosphorothioate groups.

In some embodiments region A comprises at least one phosphodiester linkage between two nucleotide analogue units, or a nucleotide analogue unit and a nucleobase unit of Region B. In some embodiments region C comprises at least one phosphodiester linkage between two nucleotide analogue units, or a nucleotide analogue unit and a nucleobase unit of Region B.

In some embodiments, region C comprises at least one phosphodiester linkage between a nucleotide analogue unit and a nucleobase unit of Region D.

In some embodiments the internucleobase linkage between the 3′ nucleotide analogue of region A and the 5′ nucleobase of region B is a phosphodiester.

In some embodiments the internucleobase linkage between the 3′ nucleobase of region B and the 5′ nucleotide analogue of region C is a phosphodiester.

In some embodiments the internucleobase linkage between the two adjacent nucleotide analogues at the 5′ end of region A are phosphodiester.

In some embodiments the internucleobase linkage between the two adjacent nucleotide analogues at the 3′ end of region C is phosphodiester.

In some embodiments the internucleobase linkage between the two adjacent nucleotide analogues at the 3′ end of region A is phosphodiester.

In some embodiments the internucleobase linkage between the two adjacent nucleotide analogues at the 5′ end of region C is phosphodiester.

In some embodiments region A has a length of 4 nucleotide analogues and the internucleobase linkage between the two middle nucleotide analogues of region A is phosphodiester.

In some embodiments region C has a length of 4 nucleotide analogues and internucleobase linkage between the two middle nucleotide analogues of region C is phosphodiester.

In some embodiments all the internucleobase linkages between nucleotide analogues present in the compound of the invention are phosphodiester.

In some embodiments, such as the embodiments referred to above, where suitable and not specifically indicated, all remaining internucleobase linkages are either phosphodiester or phosphorothioate, or a mixture thereof.

In some embodiments all the internucleobase linkage groups are phosphorothioate.

When referring to specific gapmer oligonucleotide sequences, such as those provided herein it will be understood that, in one embodiment, when the linkages are phosphorothioate linkages, alternative linkages, such as those disclosed herein may be used, for example phosphate (phosphodiester) linkages may be used, particularly for linkages between nucleotide analogues, such as LNA, units. Likewise, when referring to specific gapmer oligonucleotide sequences, such as those provided herein, when the C residues are annotated as 5′ methyl modified cytosine, in one embodiment, one or more of the Cs present in the oligonucleotide may be unmodified C residues.

### Method of Identification and Preparation of Compounds of the Invention:

The compounds of the invention, which modulate expression of the target, may be identified through experimentation or though rational design based on sequence information on the target and know-how on how best to design an oligomeric compound against a desired target. The sequences of these compounds are preferred embodiments of the invention. Likewise, the sequence motifs in the target to which these preferred oligomeric compounds are complementary (referred to as “hot spots”) are preferred sites for targeting.

In many cases the identification of an oligomeric compound, such as an LNA oligonucleotide, effective in modulating PCSK9 expression or activity in vivo or clinically is based on sequence information on the target gene (such as SEQ ID NO 2). However, one of ordinary skill in the art will appreciate that such oligomeric compounds can also be identified by empirical testing. oligomeric compounds having, for example, less sequence homology, greater or fewer modified nucleotides, or longer or shorter lengths, compared to those of the preferred embodiments, but which nevertheless demonstrate responses in clinical treatments, are also within the scope of the invention. The Examples provide suitable methods for performing empirical testing.

In a preferred embodiment, the compound of the invention comprises a subsequence or a combined contiguous nucleobase sequence which has at least 10, such as at least 11, such as at least 12, such as at least 14, such as at least 16, such as at least 18, such as 12, 13, 14, 15, 16, 17 or 18 contiguous nucleobases which are 100% complementary to both the human and mouse, or both the human and rat, or both the human and monkey, or both the human, mouse and monkey, or both the human, rat and monkey nucleic acids that encode PCSK9. In one embodiment the polynucleobase sequence of the compound is 100% complementary to both the human and mouse, or both the human and rat, or both the human and monkey, or both the human, mouse and monkey, or both the human, rat and monkey nucleic acids that encode PCSK9. In one embodiment, when referring to compounds of the invention that are 100% complementary to more than one mammalian species as listed above, one or two mismatches between 1 or more of the sequence may exist, although it is preferred that there are no mismtaches. FIG. 17 illustrates an alignment between the human and mouse nucleic acids that encode the respective human and mouse PCSK9 polypeptides. Table 1 provides suitable PCSK9 polynucleotides and the corresponding polypeptides provided by the NCBI Genbank Accession numbers—certain known allelic variants and known homologues from other mammalian species may be easily identified by performing BLAST searches using the sequences referenced in Table 1.

TABLE 1

| | Nucleic acid | Polypeptide |
|---|---|---|
| | (mRNA/cDNA sequence) | (deduced) |
| Human | NM_174936 | NP_777596 |
| Mouse | NM_153565 | NP_705793.1 |
| Rat | NM_199253 | NP_954862.2 |
| Chimpanzee | NC_006468 (genomic - | XP_001154126 |
| | mRNA annotated) | |
| Monkey (Rhesus macaque) | BV166576 | |

Amino acid and polynucleotide homology may be determined using ClustalW algorithm using standard settings: see http://www.ebi.ac.uk/emboss/align/index.html, Method: EMBOSS::water (local): Gap Open=10.0, Gap extend=0.5, using Blosum 62 (protein), or DNAfull for nucleotide sequences. As illustrated in FIG. 17, such alignments can also be used to identify regions of the nucleic acids encoding PCSK9 from human and a different mammalian species, such as monkey, mouse and/or rat, where there are sufficient stretches of nucleic acid complementarity to allow the design of oligonucleotides which target both the human PCSK9 target nucleic acid, and the corresponding nucleic acids present in the different mammalian species, such as regions of at least 10, such as at least 12, such as at least 14, such as at least 16, such as at least 18, such as 12, 13, 14, 15, 16, 17 or 18 contiguous nucleobases which are 100% complementary to both the nucleic acid encoding PCSK9 from humans and the nucleic acid(s) encoding PCSK9 from the different mammalian species.

### DEFINITIONS

When determining “homology” between the oligomeric compounds of the invention (or sub-sequence or combined contiguous nucleobase sequence) and the nucleic acid which encodes the mammalian PCSK9, such as those disclosed herein (including SEQ ID No 2), the determination of homology may be made by a simple alignment with the corresponding nucleobase sequence of the compound of the invention and the corresponding region of the nucleic acid which encodes the mammalian PCSK9 (or target nucleic acid), and the homology is determined by counting the number of bases which align and dividing by the total number of contiguous bases in the compound of the invention, and multiplying by 100. In such a comparison, if gaps exist, it is preferable that such gaps are merely mismatches rather than areas where the number of nucleobases within the gap differ between the nucleobase sequence of the invention and the target nucleic acid.

The terms “located within” and “corresponding to”/“corresponds to” refer to the comparison between the nucleobase sequence of the oligomer or contiguous nucleobase sequence and the equivalent nucleotide sequence of either the nucleic acid target such as the mRNA which encodes the PCSK9 target protein, such as SEQ ID NO 2, or the reverse complement of the nucleic acid target. Nucleotide analogues are compared directly to their equivalent or corresponding nucleotides.

The terms “corresponding nucleotide analogue” and “corresponding nucleotide” are intended to indicate that the nucleobase in the nucleotide analogue and the nucleotide are identical. For example, when the 2-deoxyribose unit of the nucleotide is linked to an adenine, the “corresponding nucleotide analogue” contains a pentose unit (different from 2-deoxyribose) linked to an adenine.

The term “continuous” in relation to a sequence of nucleobases, is interchangeable with the term “continuous”.

The term “nucleobase” is used as a collective term which encompasses both nucleotides and nucleotide analogues. A nucleobase sequence is a sequence which comprises at least two nucleotides or nucleotide analogues. In one embodiment the nucleobase sequence may comprise of only nucleotides, such as DNA units, in an alternative embodiment, the nucleobase sequence may comprise of only nucleotide analogues, such as LNA units.

The term “nucleic acid” is defined as a molecule formed by covalent linkage of two or more nucleotides.

The terms “nucleic acid” and “polynucleotide” are used interchangeable herein.

The following terms are used as they are defined in PCT/DK2006/000512: “nucleotide”, “nucleotide analogue”, “located within”, “corresponding to”/“corresponds to”, “corresponding nucleotide analogue” and “corresponding nucleotide”, “nucleobase”, “nucleic acid” and “polynucleotide”, “compound” when used in the context of a “compound of the invention”, “oligomeric compound”, “oligonucleotide”, “antisense oligonucleotide”, and “oligo”, “unit”, “LNA”, “at least one”, “linkage group”, “conjugate”, “pharmaceutically acceptable salts”, “C₁₋₄-alkyl”, “gene”, “RNA antagonist”, “mRNA”, “complementary”, “mismatche(s)”,

The term “target nucleic acid”, as used herein refers to the DNA encoding mammalian PCSK9 polypeptide, such as human PCSK9, such as SEQ ID NO 2, the mouse, rat, chimpanzee and/or monkey PCSK9 encoding nucleic acids or naturally occurring variants thereof, and RNA nucleic acids derived therefrom, preferably mRNA, such as pre-mRNA, although preferably mature mRNA. In one embodiment, for example when used in research or diagnostics the “target nucleic acid” may be a cDNA or a synthetic oligonucleotide derived from the above DNA or RNA nucleic acid targets. The oligomeric compound according to the invention is preferably capable of hybridising to the target nucleic acid.

The term “naturally occurring variant thereof” refers to variants of the PCSK9 polypeptide of nucleic acid sequence which exist naturally within the defined taxonomic group, such as mammalian, such as mouse, rat, monkey, chimpanzee and preferably human. Typically, when referring to “naturally occurring variants” of a polynucleotide the term also may encompasses variants of the PCSK9 encoding genomic DNA which are found at the NARC1 locus, or a locus directly derived from the NARC-1 locus, e.g. by chromosomal translocation or duplication, and the RNA, such as mRNA derived therefrom. When referenced to a specific polypeptide sequence, e.g. SEQ ID NO 1, the term also includes naturally occurring forms of the protein which may therefore be processed, e.g. by co- or post-translational modifications, such as signal peptide cleavage, proteolytic cleavage, glycosylation, etc.

It is preferred that the compound according to the invention is a linear molecule or is synthesised as a linear molecule.

The term “linkage group” is intended to mean a group capable of covalently coupling together two nucleotides, two nucleotide analogues, and a nucleotide and a nucleotide analogue, etc. Specific and preferred examples include phosphate groups and phosphorothioate groups.

In the present context the term “conjugate” is intended to indicate a heterogenous molecule formed by the covalent attachment of a compound as described herein (i.e. a compound comprising a sequence of nucleotides analogues) to one or more non-nucleotide/non-nucleotide-analogue, or non-polynucleotide moieties. Examples of non-nucleotide or non-polynucleotide moieties include macromolecular agents such as proteins, fatty acid chains, sugar residues, glycoproteins, polymers, or combinations thereof. Typically proteins may be antibodies for a target protein. Typical polymers may be polyethylene glycol. When the compound of the invention consists of a nucleobase sequence, it may, in one embodiment further comprise a non-nucleobase portion, such as the above conjugates.

The term “at least one” comprises the integers larger than or equal to 1, such as 1, 2, 3, 4, 5, 6, 7, 8, 9, 10, 11, 12, 13, 14, 15, 16, 17, 18, 19, 20 and so forth.

In one embodiment, such as when referring to the nucleic acid or protein targets of the compounds of the invention, the term “at least one” includes the terms “at least two” and at “least three” and “at least four”, likewise the term “at least two” may comprise the terms at “least three” and “at least four”.

As used herein, the term “pharmaceutically acceptable salts” refers to salts that retain the desired biological activity of the herein identified compounds and exhibit minimal undesired toxicological effects. Non-limiting examples of such salts can be formed with organic amino acid and base addition salts formed with metal cations such as zinc, calcium, bismuth, barium, magnesium, aluminum, copper, cobalt, nickel, cadmium, sodium, potassium, and the like, or with a cation formed from ammonia, /V,/V-dibenzylethylene-diamine, D-glucosamine, tetraethylammonium, or ethylenediamine; or (c) combinations of (a) and (b); e.g., a zinc tannate salt or the like.

In the present context, the term “C1-4-alkyl” is intended to mean a linear or branched saturated hydrocarbon chain wherein the chain has from one to four carbon atoms, such as methyl, ethyl, n-propyl, isopropyl, n-butyl, isobutyl, sec-butyl and tert-butyl.

As used herein, the term “gene” means the gene including exons, introns, non-coding 5′ and 3′ regions and regulatory elements and all currently known variants thereof and any further variants, which may be elucidated.

As used herein, the terms “RNA antagonist” refers to an oligonucleotide which targets any form of RNA (including pre-mRNA, mRNA, miRNA, siRNA etc).

The term “related disorders” when referring to hypercholesterolemia refers to one or more of the conditions selected from the group consisting of: atherosclerosis, hyperlipidemia, HDL/LDL cholesterol imbalance, dyslipidemias, e.g., familial combined hyperlipidemia (FCHL), acquired hyperlipidemia, statin-resistant hypercholesterolemia, coronary artery disease (CAD), and coronary heart disease (CHD).

In one embodiment, the term “oligomeric compound” refers to an oligonucleotide which can induce a desired therapeutic effect in humans through for example binding by hydrogen bonding to a target nucleic acid. It is also envisaged that the oligomeric compounds disclosed herein may have non-therapeutic applications, such as diagnostic applications.

As used herein, the term “modulation” means either an increase (stimulation) or a decrease (inhibition) in the expression of a gene. In the present invention, inhibition is the preferred form of modulation of gene expression and mRNA is a preferred target.

As used herein, “hybridisation” means hydrogen bonding, which may be Watson-Crick, Holstein, reversed Holstein hydrogen bonding, etc. between complementary nucleotide bases. Watson and Crick showed approximately fifty years ago that deoxyribo nucleic acid (DNA) is composed of two strands which are held together in a helical configuration by hydrogen bonds formed between opposing complementary nucleobases in the two strands. The four nucleobases, commonly found in DNA are guanine (G), adenine (A), thymine (T) and cytosine (C) of which the G nucleobase pairs with C, and the A nucleobase pairs with T. In RNA the nucleobase thymine is replaced by the nucleobase uracil (U), which similarly to the T nucleobase pairs with A. The chemical groups in the nucleobases that participate in standard duplex formation constitute the Watson-Crick face. Hoogsteen showed a couple of years later that the purine nucleobases (G and A) in addition to their Watson-Crick face have a Hoogsteen face that can be recognised from the outside of a duplex, and used to bind pyrimidine oligonucleotides via hydrogen bonding, thereby forming a triple helix structure.

It is highly preferred that the compounds of the invention are capable of hybridizing to the target nucleic acid, such as the mRNA.

In a preferred embodiment, the oligonucleotides are capable of hybridising against the target nucleic acid(s), such as the corresponding PCSK9 mRNA(s), to form a duplex with a Tₘ of at least 37° C., such as at least 40° C., at least 50° C., at least 55° C., or at least 60° C. In one aspect the Tₘ is between 37° C. and 80° C., such as between 50 and 70° C., or between 40 and 60° C., or between 40 and 70° C. In one embodiment the Tₘ is lower than 80° C., such as lower than 70° C. or lower that 60° C. or lower than 50° C.

### Measurement of Tₘ

A 3 μM solution of the compound in 10 mM sodium phosphate/100 mM NaCl/0.1 nM EDTA, pH 7.0 is mixed with its complement DNA or RNA oligonucleotide at 3 μM concentration in 10 mM sodium phosphate/100 mM NaCl/0.1 nM EDTA, pH 7.0 at 90° C. for a minute and allowed to cool down to room temperature. The melting curve of the duplex is then determined by measuring the absorbance at 260 nm with a heating rate of 1° C./min. in the range of 25 to 95° C. The Tₘ is measured as the maximum of the first derivative of the melting curve.

### Conjugates

In one embodiment of the invention the oligomeric compound is linked to ligands/conjugates, which may be used, e.g. to increase the cellular uptake of antisense oligonucleotides. PCT/DK2006/000512 provides suitable ligands and conjugates.

The invention also provides for a conjugate comprising the compound according to the invention as herein described, and at least one non-nucleotide or non-polynucleotide moiety covalently attached to said compound. Therefore, in one embodiment where the compound of the invention consists of s specified nucleic acid, as herein disclosed, the compound may also comprise at least one non-nucleotide or non-polynucleotide moiety (e.g. not comprising one or more nucleotides or nucleotide analogues) covalently attached to said compound.

### Applications

The oligomeric compounds of the present invention can be utilized for, for example, as research reagents for diagnostics, therapeutics and prophylaxis.

Some of the benefits of utilising LNA, and methods of preparing and purifying LNA and LNA oligonucleotides are disclosed in PCT/DK2006/000512.

The oligomeric compounds of the invention, such as the LNA containing oligonucleotide compounds of the present invention, can also be utilized for as research reagents for diagnostics, therapeutics and prophylaxis.

In research, such antisense oligonucleotides may be used to specifically inhibit the synthesis of PCSK9 genes in cells and experimental animals thereby facilitating functional analysis of the target or an appraisal of its usefulness as a target for therapeutic intervention.

In diagnostics the antisense oligonucleotides may be used to detect and quantitate PCSK9 expression in cell and tissues by Northern blotting, in-situ hybridisation or similar techniques.

For therapeutics, an animal or a human, suspected of having a disease or disorder, which can be treated by modulating the expression of PCSK9 is treated by administering antisense compounds in accordance with this invention. Further provided are methods of treating an animal particular mouse and rat and treating a human, suspected of having or being prone to a disease or condition, associated with expression of PCSK9 by administering a therapeutically or prophylactically effective amount of one or more of the antisense compounds or compositions of the invention.

The pharmaceutical composition according to the invention can be used for the treatment of conditions associated with abnormal levels of PCSK9, such as hypercholesteromeia and related disorders.

Suitable dosages, formulations, administration routes, compositions, dosage forms, combinations with other therapeutic agents, pro-drug formulations are also provided in PCT/DK2006/000512, although it should be recognised that the aspects of PCT/DK2006/000512 which are only specifically applicable to the treatment of cancer may not be appropriate in the therapeutic/pharmaceutical compositions and methods of the present invention.

The invention also provides for a pharmaceutical composition comprising a compound or a conjugate as herein described or a conjugate, and a pharmaceutically acceptable diluent, carrier or adjuvant. PCT/DK2006/000512 provides suitable and preferred pharmaceutically acceptable diluent, carrier and adjuvants.

Pharmaceutical Compositions Comprising More than One Active Ingredient

The pharmaceutical composition according to the invention may further comprise other active ingredients, including those which are indicated as being useful for the treatment of hypercholesterolemia and/or related disorders.

One such class of compounds are statins. The statins are HMG-CoA reductase inhibitors that form a class of hypolipidemic agents, used as pharmaceuticals to lower cholesterol levels in people at risk for cardiovascular disease because of hypercholesterolemia. They work by inhibiting the enzyme HMG-CoA reductase, the enzyme that determines the speed of cholesterol synthesis. Inhibition of this enzyme in the liver stimulates the LDL-receptors, which results in an increased clearance of LDL from the bloodstream and a decrease in blood cholesterol levels. Examples of statins include Atorvastatin™, Cerivastatin™, Fluvastatin™, Lovastatin™, Mevastatin™, Pitavastatin™, Pravastatin™, Rosuvastatin™, and Simvastatin™. The combined use of the compound of the invention and the statins may allow for a reduction in the dose of the statins, therefore overcoming side effects associated with usual dosage of statins, which include, for example, myalgias, muscle cramps, gastrointestinal symptoms, liver enzyme derangements, myositis, myopathy, rhabdomyolysis (the pathological breakdown of skeletal muscle) which may lead to acute renal failure when muscle breakdown products damage the kidney.

Fibrates, a class of amphipathic carboxylic acids is an alternative class of compound which are often combined with statin use, despite an increased frequency of rhabdomyolysis which has been reported with the combined use of statins and fibrates. The composition according to the invention may therefore further comprise fibrates, and optionally statins.

The composition according to the invention may further comprise modulators of Apolipoprotein B (Apo-B), particularly agents which are capable of lowering the expression of function of Apo-B. Suitably, the Apo-B modulators may be antisense oligonucleotides (e.g. oligomers), such as those disclosed in WO 00/97662, WO 03/11887 and WO 2004/44181. A preferred combination is with ISIS compound 301012 (illustrated as SEQ ID NO 13).

The composition according to the invention may further comprise modulators of FABP4 expression, such as antisense oligonucleotides (e.g. oligomers) which target FABP4, the composition may be used in concurrent down-regulation of both FABP4 and PSCK9 expression, resulting in a synergistic effect in terms of blood serum cholesterol and hence advantages when treating hypercholesterolemia and/or related disorders. Such compositions comprising both the compounds of the invention and FABP4 modulators, such as the antisense oligonucleotides referred to herein, may also further comprise statins. U.S. provisional application 60/969,016 hereby incorporated by reference discloses suitable FABP4 modulators.

It is also envisaged that the composition may comprise antisense oligonucleotides which comprise nucleotide analogues, such as those disclosed in PCT/DK2006/000481, which is hereby incorporated by reference. Specific LNA oligonucleotides, as disclosed or highlighted are preferred in PCT/DK2006/000481 are especially suited for us in the pharmaceutical composition according to the present invention.

The invention also provides a kit of parts wherein a first part comprises the compound, the conjugate and/or the pharmaceutical composition according to the invention and a further part comprises an antisense oligonucleotide capable of lowering the expression of Apo-B or FABP4. It is therefore envisaged that the kit of parts may be used in a method of treatment, as referred to herein, where the method comprises administering both the first part and the further part, either simultaneously or one after the other.

### Medical Methods and Use

Further conditions which may be associated with abnormal levels of PCSK9, and which, therefore may be treated using the compositions, conjugates and compounds according to the invention include disorders selected form the group consisting of: hyperlipoproteinemia, familial type 3 hyperlipoproteinemia (familial dysbetalipoproteinemia), and familial hyperalphalipoprotienemia; hyperlipidemia, mixed hyperlipidemias, multiple lipoprotein-type hyperlipidemia, and familial combined hyperlipidemia; hypertriglyceridemia, familial hypertriglyceridemia, and familial lipoprotein lipase; hypercholesterolemia, statin-resistant hypercholesterolemia familial hypercholesterolemia, polygenic hypercholesterolemia, and familial defective apolipoprotein B; cardiovascular disorders including atherosclerosis and coronary artery disease; thrombosis; peripheral vascular disease, and obesity.

Further conditions which may be associated with abnormal levels of PCSK9, and which, therefore may be treated using the compositions, conjugates and compounds according to The invention include disorders selected form the group consisting of: von Gierke's disease (glycogen storage disease, type I); lipodystrophies (congenital and acquired forms); Cushing's syndrome; sexual ateloitic dwarfism (isolated growth hormone deficiency); diabetes mellitus; hyperthyroidism; hypertension; anorexia nervosa; Werner's syndrome; acute intermittent porphyria; primary biliary cirrhosis; extrahepatic biliary 5 obstruction; acute hepatitis; hepatoma; systemic lupus erythematosis; monoclonal gammopathies (including myeloma, multiple myeloma, macroglobulinemia, and lymphoma); endocrinopathies; obesity; nephrotic syndrome; metabolic syndrome; inflammation; hypothyroidism; uremia (hyperurecemia); impotence; obstructive liver disease; idiopathic hypercalcemia; dysqlobulinemia; elevated insulin levels; Syndrome X; Dupuytren's contracture; AIDS; and Alzheimer's disease and dementia.

The invention further provides methods of inhibiting cholesterol particle binding to vascular endothelium comprising the step of administering to an individual an amount of a compound of the invention sufficient to PCSK9 expression, and as a result, the invention also provides methods of reducing the risk of: (i) cholesterol particle oxidization; (ii) monocyte binding to vascular endothelium; (iii) monocyte differentiation into macrophage; (iv) macrophage ingestion of oxidized lipid 30 particles and release of cytokines (including, but limited to IL-1, TNF-alpha, TGF-beta); (v) platelet formation of fibrous fibrofatty lesions and inflammation; (vi) endothelium lesions leading to clots; and (vii) clots leading to myocardial infarction or stroke, also comprising the step of administering to an individual an amount of a compound of the invention sufficient to inhibit PCSK9 expression.

The invention also provides methods of reducing hyperlipidemia associated with alcoholism, smoking, use of oral contraceptives, use of glucocorticoids, use of beta-adrenergic blocking agents, or use of isotretinoin (13-cis retinoic acid) comprising the step of administering to an individual an amount of a compound of the invention sufficient to inhibit PCSK9 expression.

The invention further provides use of a compound of the invention in the manufacture of a medicament for the treatment of any and all conditions disclosed herein.

Generally stated, one aspect of the invention is directed to a method of treating a mammal suffering from or susceptible to conditions associated with abnormal levels of PCSK9, comprising administering to the mammal and therapeutically effective amount of an oligonucleotide targeted to PCSK9 that comprises one or more LNA units.

An interesting aspect of the invention is directed to the use of a compound as defined herein or as conjugate as defined herein for the preparation of a medicament for the treatment of a condition according to above.

The methods of the invention are preferably employed for treatment or prophylaxis against diseases caused by abnormal levels of PCSK9.

Furthermore, the invention described herein encompasses a method of preventing or treating a disease comprising a therapeutically effective amount of a PCSK9 modulating oligonucleotide compound, including but not limited to high doses of the oligomer, to a human in need of such therapy. The invention further encompasses the use of a short period of administration of a PCSK9 modulating oligonucleotide compound.

In one embodiment of the invention the oligonucleotide compound is linked to ligands/conjugates. It is way to increase the cellular uptake of antisense oligonucleotides.

Oligonucleotide compounds of the invention may also be conjugated to active drug substances, for example, aspirin, ibuprofen, a sulfa drug, an antidiabetic, an antibacterial or an antibiotic.

Alternatively stated, the invention is furthermore directed to a method for treating abnormal levels of PCSK9, said method comprising administering a compound as defined herein, or a conjugate as defined herein or a pharmaceutical composition as defined herein to a patient in need thereof and further comprising the administration of a further chemotherapeutic agent. Said further administration may be such that the further chemotherapeutic agent is conjugated to the compound of the invention, is present in the pharmaceutical composition, or is administered in a separate formulation.

The invention also relates to a compound, composition or a conjugate as defined herein for use as a medicament.

The invention further relates to use of a compound, composition, or a conjugate as defined herein for the manufacture of a medicament for the treatment of abnormal levels of PCSK9. Typically, said abnormal levels of PCSK9 is in the form of, or causes, or is characterised by, hypercholesterolemia and related disorders, such as atherosclerosis or hyperlipidemia.

Moreover, the invention relates to a method of treating a subject suffering from a disease or condition selected from hypercholesterolemia and related disorders, such as atherosclerosis, and hyperlipidemia, the method comprising the step of administering a pharmaceutical composition as defined herein to the subject in need thereof. Preferably, the pharmaceutical composition is administered orally.

Examples of related diseases also include different types of HDL/LDL cholesterol imbalance; dyslipidemias, e.g., familial combined hyperlipidemia (FCHL), acquired hyperlipidemia, statin-resistant hypercholesterolemia; coronary artery disease (CAD) coronary heart disease (CHD), atherosclerosis.

It is recognised that when the composition according to the invention also comprises modulators of Apo-B100 or FABP4 expression, such as antisense oligonucleotides which target ApoB-100 or FABP4, the composition may be used in concurrent down-regulation of both PCSK9 and ApoB-100 (or FABP4) expression, resulting in a synergistic effect in terms of blood serum cholesterol and hence advantages when treating hypercholesterolemia and/or related disorders. Such compositions comprising both the compounds of the invention and ApoB or FABP4 modulators, such as the antisense oligonucleotides referred to herein, may also further comprise statins.

Embodiments of the Invention. The following list refer to some, non-limiting, aspects of the invention which may be combined with the other embodiments referred to in the specification and claims:
1. A compound consisting of a contiguous sequence of a total of between 10-50 nucleobases, wherein said contiguous nucleobase sequence is at least 80% homologous to a corresponding region of a nucleic acid which encodes a mammalian PCSK9.
2. A compound according to embodiment 1, wherein said compound consists of a contiguous sequence of a total of between 10-30 nucleobases, wherein said compound comprises a subsequence of at least 8 contiguous nucleobases, wherein said subsequence corresponds to a contiguous sequence which is present in the nucleic acids which encode mammalian PCSK9, wherein said subsequence may comprise no more than one mismatch when compared to the corresponding sequence present in the nucleic acid which encodes said mammalian PCSK9.
3. The compound according to embodiment 1 or 2, wherein said nucleic acid which encodes said mammalian PCSK9, is naturally present in a mammal selected form the group consisting of: a rodent, a mouse, a rat, a primate, a human, a monkey and a chimpanzee.
4. The compound according to embodiment 1 or 2, wherein said nucleic acid which encodes said mammalian PCSK9, is naturally present in a human being.
5. The compound according to any one of embodiments 2-4, wherein said compound comprises a 5′ and/or a 3′ flanking nucleobase sequence, which is/are contiguous to said subsequence, wherein said flanking sequence or sequences consist of a total of between 2 and 22 nucleobase units, which when combined with said sub-sequence, the combined contiguous nucleobase sequence is at least 80% homologous, such as at least 85% homologous, such as at least 90% homologous, such as at least 95% homologous, such as at least 97% homologous, such as 100% homologous to the corresponding sequence of said nucleic acid which encodes said mammalian PCSK9.
6. The compound according to any one of embodiments 2 to 5, wherein said subsequence or combined nucleobase sequence comprises a contiguous sequence of at least 7 nucleobase residues which, when formed in a duplex with the complementary target RNA corresponding to said nucleic acid which encodes said mammalian PCSK9, are capable of recruiting RNaseH.
7. The compound according to embodiment 6, wherein said subsequence or combined nucleobase sequence comprises of a contiguous sequence of at least 8, at least 9 or at least 10 nucleobase residues which, when formed in a duplex with the complementary target RNA corresponding to said nucleic acid which encodes said mammalian PCSK9, are capable of recruiting RNaseH.
8. The compound according to any one of the preceding embodiments wherein said subsequence is at least 9 or at least 10 nucleobases in length, such as at least 12 nucleobases or at least 14 nucleobases in length, such as 14 or 16 nucleobases in length.
9. The compound according to any one of the preceding embodiments, wherein said nucleic acid which encodes said mammalian PCSK9 is SEQ ID NO 2 or naturally occurring variant thereof.
10. The compound according to any one of the preceding embodiments, wherein said compound consists of no more than 22 nucleobases, such as no more than 20 nucleobases, such as no more than 18 nucleobases, optionally conjugated with one or more non-nucleobase compounds.
11. The compound according to embodiment 10 wherein said compound consists of either 13, 14, 15, 16 or 17 nucleobases, optionally conjugated with one or more non-nucleobase compounds.
12. The compound according to any one of the preceding embodiments wherein said compound comprises of no more than 3 mismatches with the corresponding region of the nucleic acid which encodes said mammalian PCSK9.
13. The compound according to embodiment any one of the preceding embodiments, wherein said subsequence or said combined contiguous nucleobase sequence corresponds to a sequence present in a nucleic acid sequence selected from the group consisting of SEQ ID NO 14, SEQ ID NO 15, SEQ ID No 16, SEQ ID NO 17, SEQ ID NO 18 and SEQ ID NO 19 or a sequence present in table 2, 3 and/or tables 4, 5, or 6.
14. The compound according to embodiment 13, wherein said subsequence corresponds to a sequence present in a nucleic acid sequence selected from the group consisting of SEQ ID NO 3, SEQ ID NO 4, SEQ ID NO 5, SEQ ID NO 6, SEQ ID NO 7, and SEQ ID NO 8.
15. The compound according to any one of the preceding embodiments which is an antisense oligonucleotide.
16. The compound according to embodiment 15, wherein the antisense oligonucleotide consists of a combined total of between 12 and 25 nucleobases, wherein the nucleobase sequence of said oligonucleotide is at least 80% homologous, such as at least 85% homologous, such as at least 90% homologous, such as at least 95% homologous, such as at least 97% homologous, such as 100% homologous to a corresponding region of the nucleic acid which encodes said mammalian PCSK9.
17. The compound according to any one of the preceding embodiments, wherein said compound, said subsequence, said combined contiguous nucleobase sequence and/or said flanking sequence or sequences, comprise at least one nucleotide analogue.
18. The compound according to embodiment 17, wherein said compound, said subsequence, said combined contiguous nucleobase sequence and/or said flanking sequence or sequences comprise a total of between 2 and 10 nucleotide analogues, such as between 5 and 8 nucleotide analogues.
19. The compound according to any one of the preceding embodiments, wherein the antisense oligonucleotide is a gapmer, a headmer, a tailmer or a mixmer, which comprises nucleobases which are both nucleotides and nucleotide analogues.
20. The compound according to embodiment 19, wherein said compound, said sub-sequence, or said combined contiguous nucleobase sequence is a gapmer of formula, in 5′ to 3′ direction, A-B-C, and optionally of formula A-B-C-D, wherein:
   A consists or comprises of at least one nucleotide analogue, such as between 1-6 nucleotide analogues, preferably between 2-5 nucleotide analogues, preferably 2, 3 or 4 nucleotide analogues, such as 3 or 4 consecutive nucleotide analogues and;
   B consists or comprises at least five consecutive nucleobases which are capable of recruiting RNAseH, such as between 1 and 12, or between 6-10, or between 7-9, such as 8 consecutive nucleobases which are capable of recruiting RNAseH, and;
   C consists or comprises of at least one nucleotide analogue, such as between 1-6 nucleotide analogues, preferably between 2-5 nucleotide analogues, preferably 2, 3 or 4 nucleotide analogues, such as 3 or 4 consecutive nucleotide analogues and;
   D where present, consists or comprises, preferably consists, of one or more DNA nucleotide, such as between 1-3 or 1-2 DNA nucleotides.
21. The compound according to embodiment 20, wherein:
   A Consists of 3 or 4 consecutive nucleotide analogues;
   B Consists of 8 or 9 or 10 consecutive DNA nucleotides or equivalent nucleobases which are capable of recruiting RNAseH;
   C Consists of 3 or 4 consecutive nucleotide analogues;
   D Consists, where present, of one DNA nucleotide.
22. The compound according to embodiment 20, wherein:
   A Consists of 3 consecutive nucleotide analogues;
   B Consists of 9 consecutive DNA nucleotides or equivalent nucleobases which are capable of recruiting RNAseH;
   C Consists of 3 consecutive nucleotide analogues;
   D Consists, where present, of one DNA nucleotide.
23. A compound according to embodiment 20, wherein:
   A Consists of 3 consecutive nucleotide analogues;
   B Consists of 10 consecutive DNA nucleotides or equivalent nucleobases which are capable of recruiting RNAseH;
   C Consists of 3 consecutive nucleotide analogues;
   D Consists, where present, of one DNA nucleotide.
24. The compound according to embodiments 20-23, wherein regions A and C correspond to said 5′ and said 3′ flanking regions, and region B corresponds to said sub-sequence.
25. The compound according to anyone of embodiments 20-24, wherein B comprises or consists of DNA nucleobases.
26. The compound according to any one of embodiments 17-25, wherein at least one nucleotide analogue is a Locked Nucleic Acid (LNA) unit.
27. The compound according to embodiment 26, which comprise between 1 and 10 LNA units such as between 2 and 8 nucleotide LNA units.
28. The compound according to embodiment 27 where all the nucleotide analogues present in said compound are LNA units.
29. The compound according to any one of the embodiments 26-28, wherein the LNAs are independently selected from oxy-LNA, thio-LNA, and amino-LNA, in either of the D-β and L-α configurations or combinations thereof.
30. The compound according to embodiment 29, wherein the LNAs are all β-D-oxy-LNA.
31. The compound according to any one of the preceding embodiments, wherein at least one of the nucleobases present in the nucleotides or nucleotide analogues is a modified nucleobase selected from the group consisting of 5-methylcytosine, isocytosine, pseudoisocytosine, 5-bromouracil, 5-propynyluracil, 6-aminopurine, 2-aminopurine, inosine, diaminopurine, and 2-chloro-6-aminopurine.
32. The compound according to any one of the preceding embodiments, wherein said compound hybridises with a corresponding mammalian PCSK9 mRNA with a Tₘ of at least 50° C.
33. The compound according to any one of the preceding embodiments, wherein said compound hybridises with a corresponding mammalian PCSK9 mRNA with a Tₘ of no greater than 80° C.
34. The compound according to any one of the preceding embodiments, where the nucleobase sequence consists or comprises of a sequence which is, or corresponds to, a sequence selected from the group consisting of SEQ ID NO 9, SEQ ID NO 10, SEQ ID NO 11, or a sequence present in tables 2 or 3 and/or tables 4, 5, or 6, wherein the nucleotides present in the compound may be substituted with a corresponding nucleotide analogue and wherein said compound may comprise one, two, or three mismatches against said selected sequence, and optionally, linkage groups other than phosphorothioate may be used.
35. The compound according to embodiment 34 which consists of a sequence selected from the group consisting of SEQ ID NOS SEQ ID NO 9, SEQ ID NO 10, and SEQ ID NO 11 or a sequence present in tables 2 or 3 and/or tables 4, 5, or 6.
36. A conjugate comprising the compound according to any one of the embodiments 1-35 and at least one non-nucleotide or non-polynucleotide moiety covalently attached to said compound
37. A pharmaceutical composition comprising a compound as defined in any of embodiments 1-35 or a conjugate as defined in embodiment 36, and a pharmaceutically acceptable diluent, carrier, salt or adjuvant
38. A pharmaceutical composition according to 37, wherein the compound is constituted as a pro-drug.
39. A pharmaceutical composition according to any one of embodiments 37-38, which further comprises an anti-inflamatory compounds and/or antiviral compounds.
40. The pharmaceutical composition according to embodiment 39 further comprising at least one further agent which is capable of lowering blood serum cholesterol.
41. The pharmaceutical composition according to embodiment 40, wherein the at least one further agent is a statin or a fibrogen.
42. The pharmaceutical composition according to embodiment 40 or 41, wherein the at least one further agent is a modulator of Apolipoprotein B-100 (Apo-B).
43. The pharmaceutical composition according to embodiment 42, wherein the modulator of Apo-B is an antisense oligonucleotide.
44. Use of a compound as defined in any one of the embodiments 1-35, or a conjugate as defined in embodiment 36, for the manufacture of a medicament for the treatment of hypercholesterolemia ore related disorder.
45. A method for treating hypercholesterolemia or related disorder, said method comprising administering a compound as defined in one of the embodiments 1-35, or a conjugate as defined in embodiment 36, or a pharmaceutical composition as defined in any one of the embodiments 37-43, to a patient in need thereof.
46. A method of inhibiting the expression of PCSK9 in a cell or a tissue, the method comprising the step of contacting said cell or tissue with a compound as defined in one of the embodiments 1-35, or a conjugate as defined in embodiment 36, or a pharmaceutical composition as defined in any one of the embodiments 37-43, so that expression of PCSK9 is inhibited.
47. A method of modulating expression of a PCSK9 gene comprising contacting the gene or RNA from the gene with the compound as defined in one of the embodiments 1-35, or a conjugate as defined in embodiment 36, or a pharmaceutical composition as defined in any one of the embodiments 37-43, so that gene expression is modulated.
48. A method of modulating the level of blood serum cholesterol in a mammal, the method comprising the step of contacting said cell or tissue with a compound as defined in one of the embodiments 1-35, or a conjugate as defined in embodiment 36, or a pharmaceutical composition as defined in any one of the embodiments 37-43, so that the blood serum cholesterol level is modulated.

TABLE 2
Designs of specific compounds/LNA antisense
oligonucleotides.

| | | | | |
|---|---|---|---|---|
| | | | | MOTIF |
| Comp'd | | | SEQ | SEQ |
| ID# | Length | Sequence | ID | ID |

| | | | | |
|---|---|---|---|---|
| 262 | 16 | 5′- [symbol] tₛgₛtₛcₛtₛgₛtₛgₛgₛaₛ [symbol] -3′ | 10 | 3 |
| 80 | 14 | 5′- [symbol] tₛaₛgₛaₛgₛgₛcₛaₛ [symbol] -3′ | 20 | 30 |
| 338 | 16 | 5′- [symbol] gₛtₛtₛaₛcₛaₛaₛaₛaₛgₛ [symbol] -3′ | 11 | 4 |
| 341 | 16 | 5′- [symbol] aₛtₛaₛcₛaₛcₛcₛtₛcₛcₛ [symbol] -3′ | 9 | 5 |
| 301 | 16 | 5′- [symbol] tₛcₛaₛgₛgₛgₛaₛaₛcₛcₛ [symbol] -3′ | 21 | 31 |
| 317 | 16 | 5′- [symbol] gₛaₛgₛcₛaₛgₛcₛtₛcₛaₛ [symbol] -3′ | 22 | 32 |
| 323 | 16 | 5′- [symbol] gₛgₛcₛaₛgₛcₛaₛgₛgₛaₛ [symbol] -3′ | 23 | 33 |
| 98 | 14 | 5′- [symbol] aₛcₛaₛcₛcₛtₛcₛcₛ [symbol] -3′ | 24 | 34 |
| 101 | 14 | 5′- [symbol] tₛcₛtₛgₛtₛgₛgₛaₛ [symbol] -3′ | 25 | 35 |
| 9 | 13 | 5′- [symbol] cₛtₛgₛtₛgₛgₛaₛaₛ [symbol] -3′ | 26 | 36 |
| 11 | 13 | 5′- [symbol] gₛaₛgₛgₛgₛtₛgₛcₛ [symbol] -3′ | 27 | 37 |
| 16 | 13 | 5′- [symbol] aₛaₛaₛcₛtₛcₛcₛaₛ [symbol] -3′ | 28 | 38 |
| 18 | 13 | 5′- [symbol] gₛaₛcₛaₛcₛcₛcₛtₛ [symbol] -3′ | 29 | 39 |

Note
the numbers referred to in the Examples and the FIGURES refer to the compound ID# numbers. The above table provides both compound ID NO#, and the corresponiding SEQ ID used in the sequence listing and the motif ID, also referred to in the sequence listing. Further oligomer sequence motifs according to the invention are shown in table 3.

### EXAMPLES

### Example 1

### Monomer Synthesis

The LNA monomer building blocks and derivatives thereof were prepared following published procedures and references cited therein, see:
WO 03/095467 A1
D. S. Pedersen, C. Rosenbohm, T. Koch (2002) Preparation of LNA Phosphoramidites, Synthesis 6, 802-808.
M. D. Sørensen, L. Kævrn¥, T. Bryld, A. E. Håkansson, B. Verbeure, G. Gaubert, P. Herdewijn, J. Wengel (2002) α-L-ribo-configured Locked Nucleic Acid (α-l-LNA): Synthesis and Properties, J. Am. Chem. Soc., 124, 2164-2176.
S. K. Singh, R. Kumar, J. Wengel (1998) Synthesis of Novel Bicyclo[2.2.1] Ribonucleosides: 2′-Amino- and 2′-Thio-LNA Monomeric Nucleosides, J. Org. Chem. 1998, 63, 6078-6079.
C. Rosenbohm, S. M. Christensen, M. D. Sørensen, D. S. Pedersen, L. E. Larsen, J. Wengel, T. Koch (2003) Synthesis of 2′-amino-LNA: a new strategy, Org. Biomol. Chem. 1, 655-663.
D. S. Pedersen, T. Koch (2003) Analogues of LNA (Locked Nucleic Acid). Synthesis of the 2′-Thio-LNA Thymine and 5-Methyl Cytosine Phosphoramidites, Synthesis 4, 578-582.

### Example 2

### Oligonucleotide Synthesis

Oligonucleotides were synthesized using the phosphoramidite approach on an Expedite 8900/MOSS synthesizer (Multiple Oligonucleotide Synthesis System) at 1 μmol or 15 μmol scale. For larger scale synthesis an Äkta Oligo Pilot was used. At the end of the synthesis (DMT-on), the oligonucleotides were cleaved from the solid support using aqueous ammonia for 1-2 h at room temperature, and further deprotected for 4 h at 65° C. The oligonucleotides were purified by reverse phase HPLC (RP-HPLC). After the removal of the DMT-group, the oligonucleotides were characterized by AE-HPLC, RP-HPLC, and CGE and the molecular mass was further confirmed by ESI-MS. See below for more details.

### Preparation of the LNA-Solid Support:

### Preparation of the LNA Succinyl Hemiester

5′-O-Dmt-3′-hydroxy-LNA monomer (500 mg), succinic anhydride (1.2 eq.) and DMAP (1.2 eq.) were dissolved in DCM (35 mL). The reaction was stirred at room temperature overnight. After extractions with NaH₂PO₄ 0.1 M pH 5.5 (2×) and brine (1×), the organic layer was further dried with anhydrous Na₂SO₄ filtered and evaporated. The hemiester derivative was obtained in 95% yield and was used without any further purification.

### Preparation of the LNA-Support

The above prepared hemiester derivative (90 μmol) was dissolved in a minimum amount of DMF, DIEA and pyBOP (90 μmol) were added and mixed together for 1 min. This pre-activated mixture was combined with LCAA-CPG (500 Å, 80-120 mesh size, 300 mg) in a manual synthesizer and stirred. After 1.5 h at room temperature, the support was filtered off and washed with DMF, DCM and MeOH. After drying, the loading was determined to be 57 μmol/g (see Tom Brown, Dorcas J. S. Brown. Modern machine-aided methods of oligodeoxyribonucleotide synthesis. In: F. Eckstein, editor. Oligonucleotides and Analogues A Practical Approach. Oxford: IRL Press, 1991: 13-14).

### Elongation of the Oligonucleotide

The coupling of phosphoramidites (A(bz), G(ibu), 5-methyl-C(bz)) or T-β-cyanoethyl-phosphoramidite) is performed by using a solution of 0.1 M of the 5′-O-DMT-protected amidite in acetonitrile and DCI (4,5-dicyanoimidazole) in acetonitrile (0.25 M) as activator. The thiolation is carried out by using xanthane chloride (0.01 M in acetonitrile:pyridine 10%). The rest of the reagents are the ones typically used for oligonucleotide synthesis. The protocol provided by the supplier was conveniently optimised.

### Purification by RP-HPLC:

### Column: Xterra RP₁₈

Flow rate: 3 mL/min
Buffers: 0.1 M ammonium acetate pH 8 and acetonitrile

### Abbreviations

### DMT: Dimethoxytrityl

### DCI: 4,5-Dicyanoimidazole

### DMAP: 4-Dimethylaminopyridine

### DCM: Dichloromethane

### DMF: Dimethylformamide

### THF: Tetrahydrofurane

### DIEA: N,N-diisopropylethylamine

PyBOP: Benzotriazole-1-yl-oxy-tris-pyrrolidino-phosphonium hexafluorophosphate

### Bz: Benzoyl

### Ibu: Isobutyryl

### Example 3

### Design of the Oligonucleotide Compound

See table 2 and 3 (below)—Upper case letters indicates ribonucleotide units and subscript “s” represents 2′-O-methyl-modified ribonucleotide units.

In one embodiment of the invention, SEQ ID NOs: 3 and 4 contains at least 3 LNA nucleotides, such as 6 (7 or 8 LNAs) nucleotides like in SEQ ID NOs: 3 and 4.

### Example 4

### Stability of LNA Compounds in Human or Rat Plasma

LNA oligonucleotide stability was tested in plasma from humans or rats (it could also be mouse, monkey or dog plasma). In 45 μl plasma 5 μl oligonucleotide is added (a final concentration of 20 μM). The oligos are incubated in plasma for times ranging from 0 h-96 h at 37° C. (the plasma is tested for nuclease activity up to 96 h and shows no difference in nuclease cleavage-pattern). At the indicated time the sample were snap-frozen in liquid nitrogen. 2 μl (equals 40 pmol) oligonucleotide in plasma was diluted by adding 15 μl of water and 3 μl 6× loading dye (Invitrogen). As marker a 10 by ladder (Invitrogen 10821-015) is used. To 1 μl ladder 1 μl 6× loading and 4 μl water was added. The samples were mixed, heated to 65° C. for 10 min and loaded to a pre-run gel (16% acrylamide, 7 M UREA, 1×TBE, pre-run at 50 Watt for 1 h) and run at 50-60 Watt for 2½ h. Subsequently the gel was stained with 1× SyBR gold (molecular probes) in 1×TBE for 15 min. The bands were visualised using a phosphoimager from Biorad.

### Example 5

### In Vitro Model: Cell Culture

The effect of antisense compounds on target nucleic acid expression can be tested in any of a variety of cell types provided that the target nucleic acid is present at measurable levels.

Target can be expressed endogenously or by transient or stable transfection of a nucleic acid encoding said nucleic acid.

The expression level of target nucleic acid can be routinely determined using, for example, Northern blot analysis, Quantitative PCR, Ribonuclease protection assays. The following cell types are provided for illustrative purposes, but other cell types can be routinely used, provided that the target is expressed in the cell type chosen.

Cells were cultured in the appropriate medium as described below and maintained at 37° C. at 95-98% humidity and 5% CO₂. Cells were routinely passaged 2-3 times weekly.

Huh-7: Human liver cell line Huh-7 was purchased from ATCC and cultured in Eagle MEM (Sigma) with 10% FBS+Glutamax I+non-essential amino acids+gentamicin.

### Example 6

### In Vitro Model: Treatment with Antisense Oligonucleotide

Cell culturing and transfection: Huh-7 and Hepa 1-6 cells were seeded in 6-well plates at 37° C. (5% CO₂) in growth media supplemented with 10% FBS, Glutamax I and Gentamicin. When the cells were 60-70% confluent, they were transfected in duplicates with different concentrations of oligonucleotides (0.04-25 nM) using Lipofectamine 2000 (5 μg/mL). Transfections were carried out essentially as described by Dean et al. (1994, JBC 269:16416-16424). In short, cells were incubated for 10 min. with Lipofectamine in OptiMEM followed by addition of oligonucleotide to a total volume of 0.5 mL transfection mix per well. After 4 hours, the transfection mix was removed, cells were washed and grown at 37° C. for approximately 20 hours (mRNA analysis and protein analysis in the appropriate growth medium. Cells were then harvested for protein and RNA analysis.

### Example 7

### In Vitro Model: Extraction of RNA and cDNA Synthesis

### Total RNA Isolation

Total RNA was isolated using RNeasy mini kit (Qiagen). Cells were washed with PBS, and Cell Lysis Buffer (RTL, Qiagen) supplemented with 1% mercaptoethanol was added directly to the wells. After a few minutes, the samples were processed according to manufacturer's instructions.

### First Strand Synthesis

First strand synthesis was performed using either OmniScript Reverse Transcriptase kit or M-MLV Reverse transcriptase (essentially as described by manufacturer (Ambion)) according to the manufacturer's instructions (Qiagen). When using OmniScript Reverse Transcriptase 0.5 μg total RNA each sample, was adjusted to 12 μl and mixed with 0.2 μl poly (dT)₁₂₋₁₈ (0.5 μg/μl) (Life Technologies), 2 μl dNTP mix (5 mM each), 2 μl 10×RT buffer, 0.5 μl RNAguard™ RNase Inhibitor (33 units/mL, Amersham) and 1 μl OmniScript Reverse Transcriptase followed by incubation at 37° C. for 60 min. and heat inactivation at 93° C. for 5 min.

When first strand synthesis was performed using random decamers and M-MLV-Reverse Transcriptase (essentially as described by manufacturer (Ambion)) 0.25 μg total RNA of each sample was adjusted to 10.8 μl in H₂O. 2 μl decamers and 2 μl dNTP mix (2.5 mM each) was added. Samples were heated to 70° C. for 3 min. and cooled immediately in ice water and added 3.25 μl of a mix containing (2 μl 10×RT buffer; 1 μl M-MLV Reverse Transcriptase; 0.25 μl RNAase inhibitor). cDNA is synthesized at 42° C. for 60 min followed by heating inactivation step at 95° C. for 10 min and finally cooled to 4° C.

### Example 8

### In Vitro and In Vivo Model: Analysis of Oligonucleotide Inhibition of PCSK9 Expression by Real-Time PCR

Antisense modulation of PCSK9 expression can be assayed in a variety of ways known in the art. For example, PCSK9 mRNA levels can be quantitated by, e.g., Northern blot analysis, competitive polymerase chain reaction (PCR), or real-time PCR. Real-time quantitative PCR is presently preferred. RNA analysis can be performed on total cellular RNA or mRNA.

Methods of RNA isolation and RNA analysis such as Northern blot analysis is routine in the art and is taught in, for example, Current Protocols in Molecular Biology, John Wiley and Sons.

Real-time quantitative (PCR) can be conveniently accomplished using the commercially iQ Multi-Color Real Time PCR Detection System available from BioRAD. Real-time Quantitative PCR is a technique well known in the art and is taught in for example Heid et al. Real time quantitative PCR, Genome Research (1996), 6: 986-994.

Real-Time Quantitative PCR Analysis of PCSK9 mRNA Levels

To determine the relative human PCSK9 mRNA level in treated and untreated samples, the generated cDNA was used in quantitative PCR analysis using an iCycler from Bio-Rad or 7500 Fast Real-Time PCR System from Applied Biosystems.

8 μl of 10-fold diluted cDNA was added 52 μl of a mix containing 29.5 μl Platinum qPCR Supermix-UDG (Invitrogen) 19.2 μl H₂O and 3.0 μl of a 20× human PCSK9 or GAPDH TaqMan gene expression assay (Applied Biosystems). Each sample was analysed in duplicates. PCR program: 95° C. for 20 seconds followed by 40 cycles of 95° C., 3 seconds, 60° C., 30 seconds.

Mouse PCSK9: Mouse PCSK9 expression is quantified using a mouse PCSK9 or GAPDH TaqMan gene expression assay (Applied Biosystems) 8 μl of 10-fold diluted cDNA is added 52 μl of a mix containing 29.5 μl Platinum qPCR Supermix-UDG (Invitrogen) 19.2 μl H₂O and 3.0 μl of a 20× mouse PCSK9 or GAPDH TaqMan gene expression assay (Applied Biosystems). Each sample is analysed in duplicates. PCR program: 95° C. for 20 seconds followed by 40 cycles of 95° C., 3 seconds, 60° C., 30 seconds.

PCSK9 mRNA expression is normalized to mouse Gapdh mRNA which was similarly quantified using Q-PCR.

2-fold dilutions of cDNA synthesised from untreated human Hepatocyte cell line (Huh-7) (diluted 5 fold and expressing both PCSK9 and Gapdh) is used to prepare standard curves for the assays. Relative quantities of PCSK9 mRNA were determined from the calculated Threshold cycle using the iCycler iQ Real Time Detection System software.

### Example 9

### In Vitro Analysis: Dose Response in Cell Culture (Human Hepatocyte Huh-7)/Antisense Inhibition of Human PCSK9 Expression

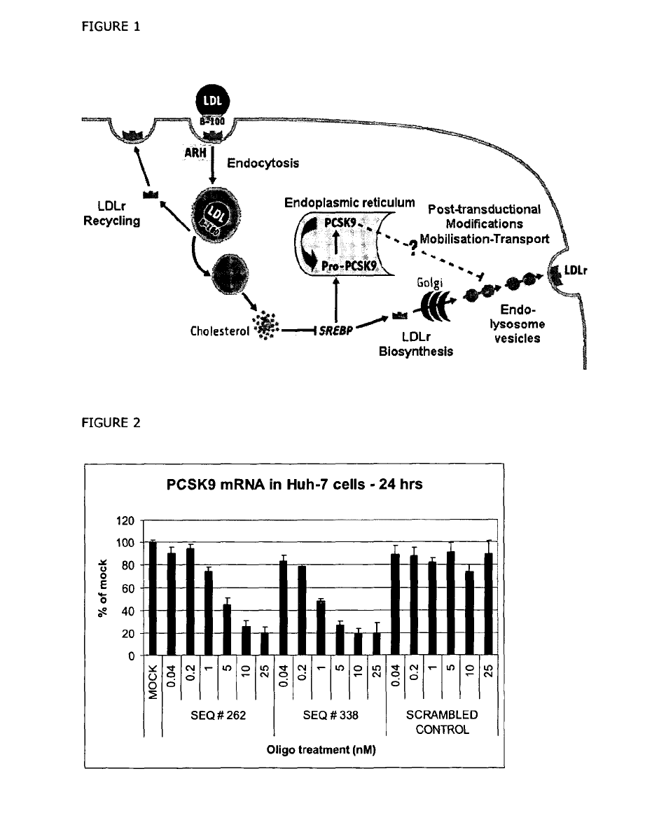

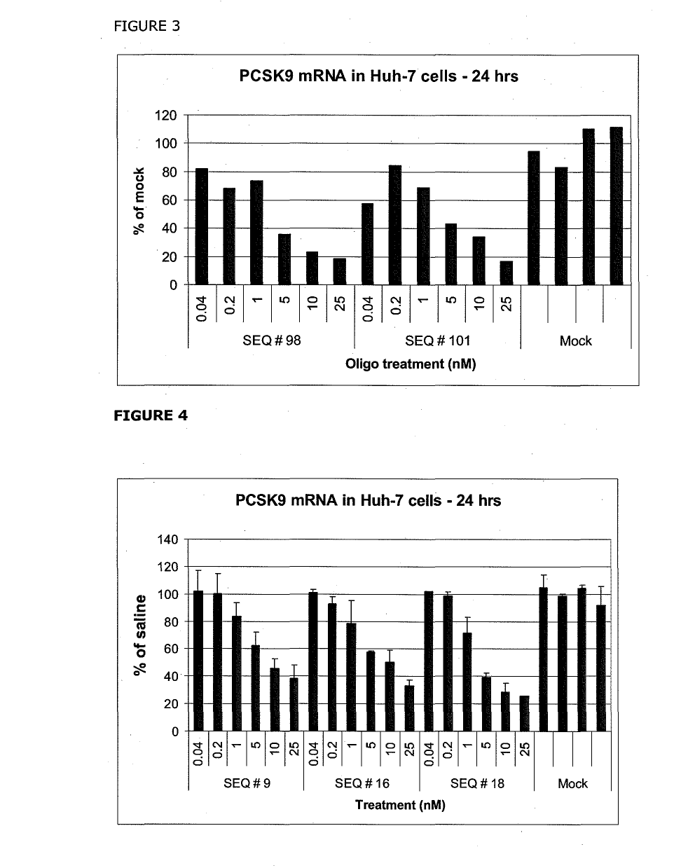

In accordance with the present invention, a series of oligonucleotides were designed to target different regions of the human PCSK9 mRNA. See Table 2 Oligonucleotide compounds were evaluated for their potential to knockdown PCSK9 mRNA in Human hepatocytes (Huh-7 cells) following lipid-assisted uptake of Compound ID NO#s: 9, 16, 18, 98, 101, 262, 301, 317, 323, 338, 341 (FIGS. 1-4). The experiment was performed as described in examples 5-8. The results showed very potent down regulation (60 to ≧80%) with 25 nM for all compounds.

### Example 10

### In Vitro Analysis: Dose Response in Cell Culture (Murine Hepatocyte Hepa 1-6)/Antisense Inhibition of Murine PCSK9 Expression

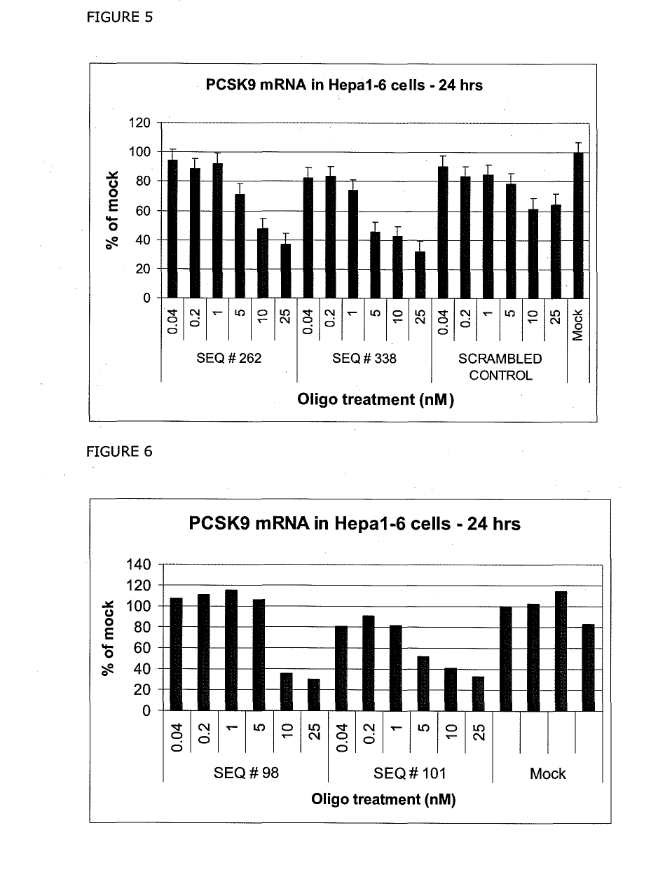

In accordance with the present invention, a series of oligonucleotides were designed to target different regions of the murine PCSK9 mRNA (See Table 2). Oligonucleotide compounds were evaluated for their potential to knockdown PCSK9 mRNA in Murine hepatocytes (Hepa 1-6) following lipid-assisted uptake of Compound ID NO#s: 98, 101, 262 and 338 (FIGS. 5-6).

The experiment was performed as described in examples 5-8. The results showed very potent down regulation (60%) with 25 nM for all compounds

### Example 11

### Cholesterol Levels in Mouse Serum

Total cholesterol level was measured in serum using a colometric assay Cholesterol CP from ABX Pentra. The cholesterol is measured following enzymatic hydrolysis and oxidation. 20 μL water was added to 3 μL serum. 240 μL reagent is added and within 15 min the cholesterol content is measured at a wavelength of 500 nM. Measurements on each animal was made in duplicates. A standard curve was made using Multi Cal from ABX Diagnostics.

Cholesterol levels in the different lipoprotein classes (VLDL/LDL and HDL) was measured in serum by ultracentrifugation. The serum was adjusted to a density of 1.067 g/ml allowing to separate HDL from the other lipoproteins. Total cholesterol (ABX Pentra) is measured in each fraction (top and bottom) after centrifugation at approximately 400.000 g for 4 hours at 15° C.

### Example 12

### LDL-Receptor Protein Level in Mouse Liver

### Western Blotting

Liver samples were snap frozen in liquid nitrogen and stored at −80° C. until analysed. 30 mg tissue was defrosted and homogenised in 300 μl T-per Tissue Protein Extraction buffer (Pierce), supplemented with Halt Protease inhibitor cocktail (Pierce).

Total protein was measured by BCA protein assay kit (Pierce) using an albumin standard according to manufacturer's protocol.

25 μg total protein from each sample was loaded on a 4-12% Bis-Tris gel with 4×LDS sample buffer (NuPAGE, Invitrogen). The gel was run for two hours at 130 V in MOPS (Invitrogen). Protein bands were blotted on a PVDF membrane using a blotting module according to standard protocol (XCell II Blot Module, Invitrogen). The membrane was blocked in 5% skimmed milk powder in 1×PBS over night. For immunodetection, the membrane was incubated overnight in a blocking solution with primary antibodies of 1:1000 dilution of polyclonal goat-anti-mouse-LDLR antibody (R&D Systems) and 1:2000 dilution of monoclonal Mouse-anti-tubulin antibody (NeoMarkers). This was followed by two hours incubation in secondary antibody solution of 1:2000 dilution of HRP/anti-goat antibody and 1:2000 dilution of HRP/anti-mouse antibody (Dako). LDLR and tubulin bands were visualized using Chemiluminescence ECL+ detection kit (Amersham) and a VersaDoc5000 imaging system (Bio-Rad).

### Example 13

### Lipoprotein Class Composition in Serum Measured Using Sebia Gels

Agarose gel electrophoresis in barbital buffer is used to separate lipoproteins according to charge and is one of the original methods for clinical analysis of lipoprotein profiles. Gels are usually stained with a lipophilic dye such as Sudan Black. The dye(s) will not distinguish between lipid species, hence the method is limited to providing a “general lipoproteins profile” as dyes cannot distinguish between cholesterol ester and triglycerides. However, the small sample volume, high reproducibility, and the possibility to follow changes in lipoprotein profiles (as percent lipid/band) in individual animals makes the agarose gels a useful tool for lipoprotein analysis. Analyses are made on high-quality gels and specialized electrophoresis equipment (Lipoprotein+Lp(a) agarose gel electrophoresis, Sebia, France). Serum was isolated from mouse blood by centrifugation and the lipoproteins were separated on Sebia Gels and quantified using Sudan Black staining followed by scanning the gels (Molecular Imager FX) and analyzed by Quantity One software, using the Densiometry settings.

### Example 14

### In Vivo Analysis: Dose Response of Different LNA Oligonucleotides in C57BL/6 Female Mice

In accordance with the present invention, a series of oligonucleotides were designed to target different regions of the murine PCSK9 mRNA. Three of these oligonucleotides were evaluated for their potential down regulation on PCSK9 mRNA in liver, reduction of serum cholesterol and increase in LDL-receptor protein in liver.

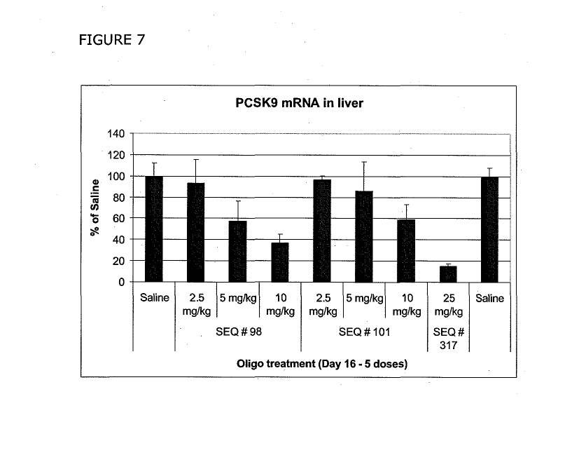

C57BL/6 female were dosed 2.5, 5, or 10 mg/kg i.v. of the oligonucleotide or saline days 0, 3, 7, 10 and 14 and sacrificed day 16 after the first treatment dose. Liver was sampled for analysis of PCSK9 mRNA expression by qPCR (as described in example 8). PCSK9 mRNA expression was down regulated in a dose dependent manner after dosing Compound ID NO#s 98 and 101 (FIG. 7).

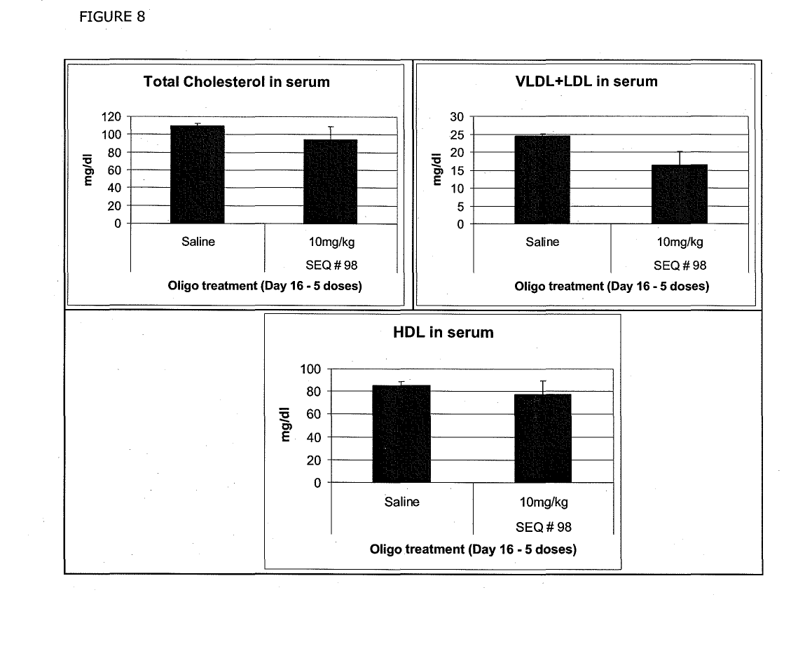

Blood was sampled at sacrifice for serum preparation and serum cholesterol was measured as described in example 11. Compound ID NO #98 showed tendency of reduced serum total cholesterol and reduced level of VLDL+LDL cholesterol, about 30% and no effect on HDL-cholesterol (FIG. 8).

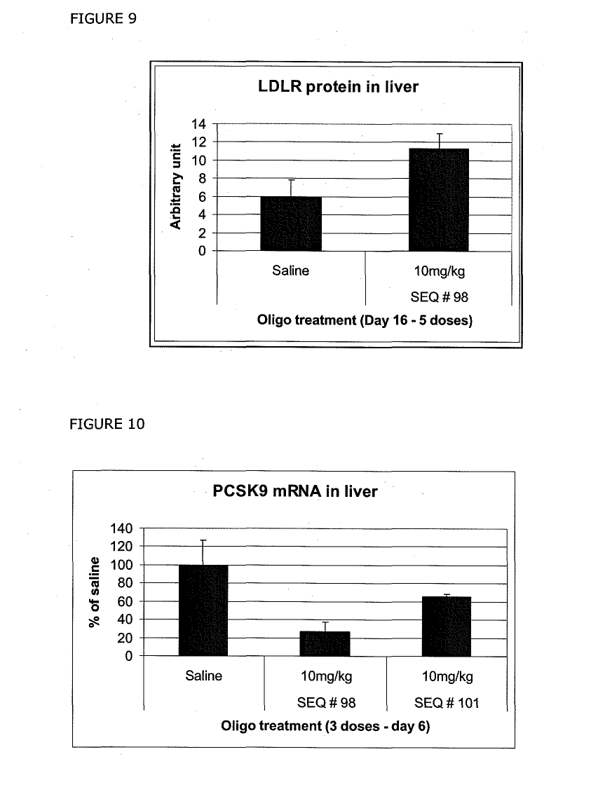

The down regulation of PCSK9 mRNA was expected to have an effect on the number of LDL-receptors presented on the surface of the hepatocytes. Western Blotting was used to examine the LDL-receptor protein in liver (example 12). The Compound ID NO #98 resulted in an increase in LDL-receptor protein of about 80% compared to the saline group (FIG. 9).

### Example 15

### In Vivo Analysis: Efficacy of LNA Oligonucleotides of Down Regulating PCSK9 in Female NMRI Mice

Two oligonucleotides targeting different refions of the murine PCSK9 mRNA was examined for potency to down regulate PCSK9 mRNA expression, reduce serum total cholesterol and increase LDL-receptor protein level.

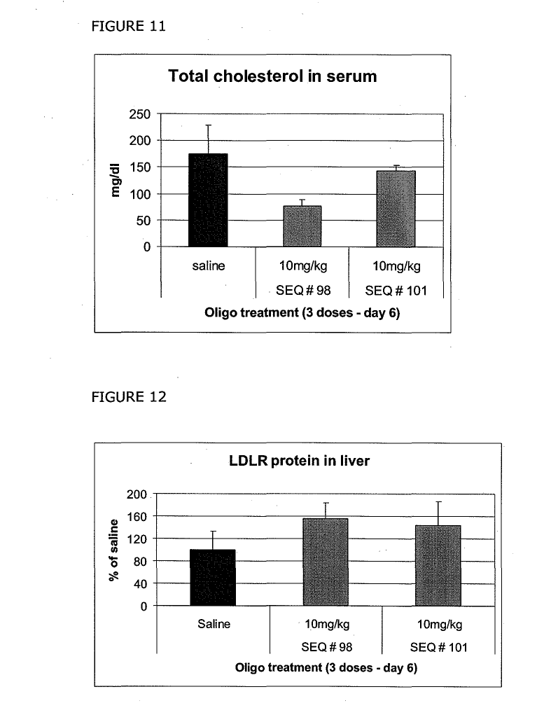

NMRI female mice were dosed i.v. 10 mg/kg/dose LNA oligonucleotide or saline at days 0, 2, 4 and sacrificed at day 6. Liver was sampled for analysis of PCSK9 mRNA expression by qPCR (as described in example 8). PCSK9 mRNA expression was reduced with about 70% after dosing Compound ID NO #98 and about 30% dosing Compound ID NO #101 (FIG. 10). The effect of this down regulation was observed on the LDL-receptor protein level in liver; about 50% and 40% increase after dosing Compound ID NO#s 98 and 101, respectively (FIG. 11. This increase in LDL-receptor resulted in decrease in serum cholesterol of 55% and 15% for Compound ID NO #98 and 101, respectively (FIG. 12).

### Example 16

### In Vivo Analysis: Efficacy of LNA Oligonucleotides to Reduce PCSK9 mRNA Expression in C57BL/6 Fed a High Fat Diet (HFD) for 1 or 5 Month Before Dosing

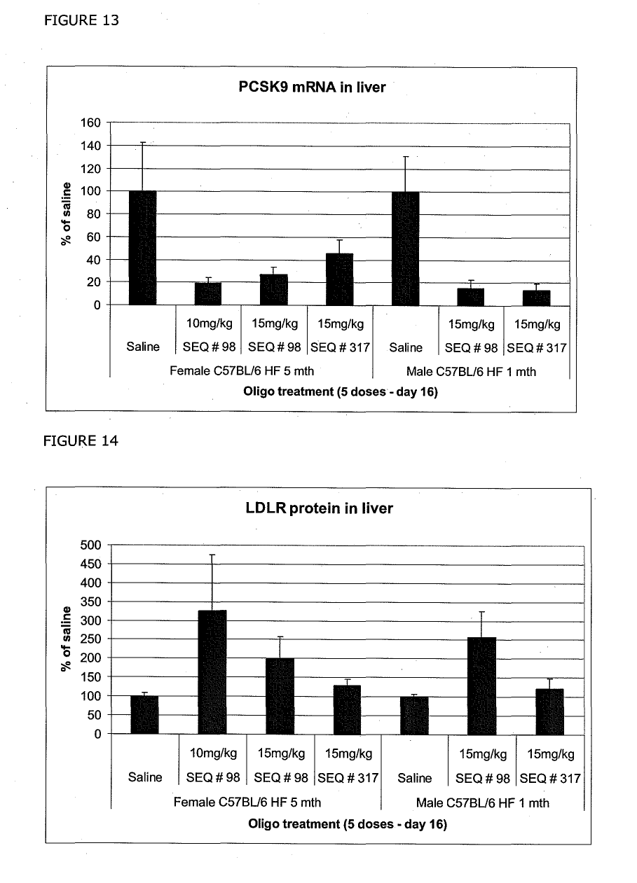

C57BL/6 female mice were fed a high fat diet (HFD) (60 energy % fat) for 5 month and male C57BL/6 were fed a HFD for 1 month before dosing LNA oligonucleotides at 10 or 15 mg/kg days 0, 3, 7, 10, 14 and sacrifice day 16. Liver was sampled for analysis of PCSK9 mRNA expression by qPCR (as described in example 8). Dosing Compound ID NO#s 98 and 317 resulted in a down regulation of PCSK9 mRNA expression (analysed by qPCR as described in example 8) of about 80 and 60%, respectively, in female mice and about 85% in male mice for both compounds (FIG. 13). The LDL-receptor protein level measured by Western blotting (described in example 12) was increased about 2-3 times after dosing Compound ID NO #98 and 20% after dosing Compound ID NO #317 to female HFD mice. In male mice 15 mg/kg/dose Compound ID NO #98 resulted in an increased LDL-receptor protein level of 2.5 times whereas Compound ID NO #317 had only minor effect on LDL-receptor protein level (FIG. 14).

### Example 17

### In Vivo Analysis: Efficacy of 13-mer LNA Oligonucleotides to Reduce PCSK9 mRNA Expression in NMRI Female Mice

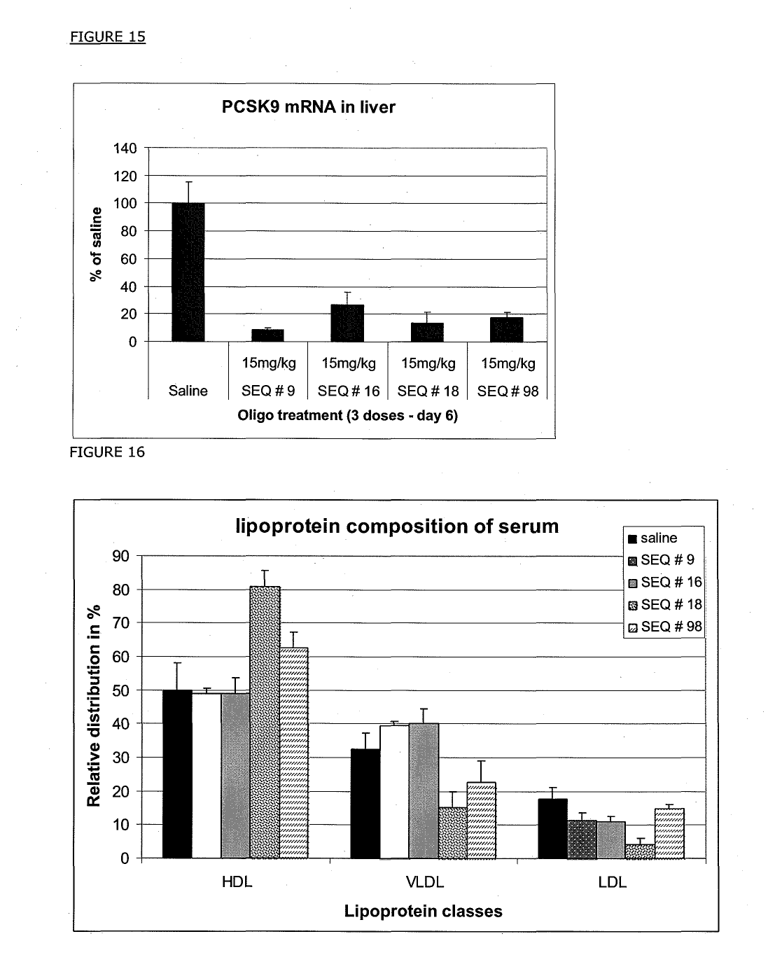

MNRI female mice were dosed 15 mg/kg days 0, 2 and 4 and sacrificed day 6. Liver was sampled for analysis of PCSK9 mRNA expression by qPCR as described in example 8. The 13-mer oligonucleotides; Compound ID NO#s 9, 16 and 18 resulted in reduction of PCSK9 mRNA expression of 90%, 70% and 85%, respectively and the 14-mer Compound ID NO #98 gave 80% reduction in PCSK9 mRNA (FIG. 15). The distribution of the different lipoprotein classes in serum was determined after separation on Sebia gels as described in example 13. The distribution between the different classes (set to 100% for each group and presented relative to the other lipoproteins in that group) was examined for Compound ID NO#s 9, 16, 18 and 98. The highest effect was observed for the Compound ID NO #18 for all lipipoproteins (50% and 65% reduction relative to saline for VLDL and LDL, respectively, as a result HDL was increased by 60%) and, Compound ID NO #98 reduced VLDL by 30% and by about 10% for LDL relative to saline, and as a result HDL was increased by 20% (FIG. 16).

TABLE 3

Further sequences of major interest for synthesis of
antisense oligonucleotides targeting PCSK9, such as LNA
antisense oligonucleotides.

| | | | | | | | |
|---|---|---|---|---|---|---|---|
| Target | Target | | | Human | Murine | | Motif |
| site | site | Oligo | | mRNA | mRNA | | Seq ID |
| Start | End | length | Target sequence | targets | targets | Oligo sequence | # |

| | | | | | | | |
|---|---|---|---|---|---|---|---|
| 1082 | 1093 | 12 | CGCTTCCACAGA | 2 | 2 | TCTGTGGAAGCG | 40 |
| 1228 | 1239 | 12 | CACCCTCATAGG | 2 | 2 | CCTATGAGGGTG | 41 |
| 1244 | 1255 | 12 | GAGTTTATTCGG | 2 | 2 | CCGAATAAACTC | 42 |
| 1239 | 1250 | 12 | GCCTGGAGTTTA | 2 | 3 | TAAACTCCAGGC | 43 |
| 1138 | 1149 | 12 | GGTCAGCGGCCG | 2 | 4 | CGGCCGCTGACC | 44 |
| 1233 | 1244 | 12 | TCATAGGCCTGG | 2 | 4 | CCAGGCCTATGA | 45 |
| 1230 | 1241 | 12 | CCCTCATAGGCC | 3 | 1 | GGCCTATGAGGG | 46 |
| 1082 | 1094 | 13 | CGCTTCCACAGAC | 1 | 1 | GTCTGTGGAAGCG | 47 |
| 1139 | 1151 | 13 | GTCAGCGGCCGGG | 1 | 1 | CCCGGCCGCTGAC | 48 |
| 1224 | 1236 | 13 | GCGGCACCCTCAT | 1 | 1 | ATGAGGGTGCCGC | 49 |
| 1228 | 1240 | 13 | CACCCTCATAGGC | 1 | 1 | GCCTATGAGGGTG | 50 |
| 1232 | 1244 | 13 | CTCATAGGCCTGG | 1 | 1 | CCAGGCCTATGAG | 51 |
| 1235 | 1247 | 13 | ATAGGCCTGGAGT | 1 | 1 | ACTCCAGGCCTAT | 52 |
| 1238 | 1250 | 13 | GGCCTGGAGTTTA | 1 | 1 | TAAACTCCAGGCC | 53 |
| 1239 | 1251 | 13 | GCCTGGAGTTTAT | 1 | 1 | ATAAACTCCAGGC | 54 |
| 1826 | 1838 | 13 | GCACACTCGGGGC | 1 | 1 | GCCCCGAGTGTGC | 55 |
| 1989 | 2001 | 13 | GTGAGGGTGTCTA | 1 | 1 | TAGACACCCTCAC | 56 |
| 844 | 856 | 13 | GAAGTTGCCCCAT | 1 | 1 | ATGGGGCAACTTC | 57 |
| 979 | 991 | 13 | GGAGGTGTATCTC | 1 | 1 | GAGATACACCTCC | 58 |
| 1233 | 1245 | 13 | TCATAGGCCTGGA | 1 | 2 | TCCAGGCCTATGA | 59 |
| 1827 | 1839 | 13 | CACACTCGGGGCC | 1 | 2 | GGCCCCGAGTGTG | 60 |
| 1231 | 1243 | 13 | CCTCATAGGCCTG | 1 | 3 | CAGGCCTATGAGG | 61 |
| 978 | 990 | 13 | TGGAGGTGTATCT | 1 | 3 | AGATACACCTCCA | 62 |
| 1603 | 1615 | 13 | TGCTGCCCACGTG | 1 | 4 | CACGTGGGCAGCA | 63 |
| 1100 | 1112 | 13 | AGCAAGTGTGACA | 2 | 1 | TGTCACACTTGCT | 64 |
| 1140 | 1152 | 13 | TCAGCGGCCGGGA | 2 | 1 | TCCCGGCCGCTGA | 65 |
| 1225 | 1237 | 13 | CGGCACCCTCATA | 2 | 1 | TATGAGGGTGCCG | 66 |
| 1226 | 1238 | 13 | GGCACCCTCATAG | 2 | 1 | CTATGAGGGTGCC | 67 |
| 1227 | 1239 | 13 | GCACCCTCATAGG | 2 | 1 | CCTATGAGGGTGC | 68 |
| 1229 | 1241 | 13 | ACCCTCATAGGCC | 2 | 1 | GGCCTATGAGGGT | 69 |
| 1230 | 1242 | 13 | CCCTCATAGGCCT | 2 | 1 | AGGCCTATGAGGG | 70 |
| 1234 | 1246 | 13 | CATAGGCCTGGAG | 2 | 1 | CTCCAGGCCTATG | 71 |
| 1244 | 1256 | 13 | GAGTTTATTCGGA | 2 | 1 | TCCGAATAAACTC | 72 |
| 1388 | 1400 | 13 | AACTTCCGGGACG | 2 | 1 | CGTCCCGGAAGTT | 73 |
| 2929 | 2941 | 13 | GGCTCCCTGATTA | 2 | 1 | TAATCAGGGAGCC | 74 |
| 843 | 855 | 13 | TGAAGTTGCCCCA | 2 | 1 | TGGGGCAACTTCA | 75 |
| 845 | 857 | 13 | AAGTTGCCCCATG | 2 | 1 | CATGGGGCAACTT | 76 |
| 1137 | 1149 | 13 | TGGTCAGCGGCCG | 2 | 2 | CGGCCGCTGACCA | 77 |
| 1138 | 1150 | 13 | GGTCAGCGGCCGG | 2 | 2 | CCGGCCGCTGACC | 78 |
| 1243 | 1255 | 13 | GGAGTTTATTCGG | 2 | 2 | CCGAATAAACTCC | 79 |
| 1581 | 1593 | 13 | CACAGAGTGGGAC | 2 | 2 | GTCCCACTCTGTG | 80 |
| 1747 | 1759 | 13 | GACCCCCAACCTG | 2 | 2 | CAGGTTGGGGGTC | 81 |
| 2466 | 2478 | 13 | CCATCTGCTGCCG | 2 | 2 | CGGCAGCAGATGG | 82 |
| 1986 | 1998 | 13 | GGGGTGAGGGTGT | 2 | 3 | ACACCCTCACCCC | 83 |
| 2468 | 2480 | 13 | ATCTGCTGCCGGA | 2 | 3 | TCCGGCAGCAGAT | 84 |
| 976 | 988 | 13 | GGTGGAGGTGTAT | 2 | 3 | ATACACCTCCACC | 85 |
| 1085 | 1097 | 13 | TTCCACAGACAGG | 2 | 4 | CCTGTCTGTGGAA | 86 |
| 1086 | 1098 | 13 | TCCACAGACAGGC | 2 | 4 | GCCTGTCTGTGGA | 87 |
| 1245 | 1257 | 13 | AGTTTATTCGGAA | 2 | 4 | TTCCGAATAAACT | 88 |
| 1434 | 1446 | 13 | AGGTCATCACAGT | 2 | 4 | ACTGTGATGACCT | 89 |
| 1389 | 1401 | 13 | ACTTCCGGGACGA | 2 | 5 | TCGTCCCGGAAGT | 90 |
| 1580 | 1592 | 13 | TCACAGAGTGGGA | 2 | 6 | TCCCACTCTGTGA | 91 |
| 1240 | 1252 | 13 | CCTGGAGTTTATT | 3 | 1 | AATAAACTCCAGG | 92 |
| 1410 | 1422 | 13 | TCTACTCCCCAGC | 3 | 1 | GCTGGGGAGTAGA | 93 |
| 2930 | 2942 | 13 | GCTCCCTGATTAA | 3 | 1 | TTAATCAGGGAGC | 94 |
| 1082 | 1095 | 14 | CGCTTCCACAGACA | 1 | 1 | TGTCTGTGGAAGCG | 95 |
| 1084 | 1097 | 14 | CTTCCACAGACAGG | 1 | 1 | CCTGTCTGTGGAAG | 96 |
| 1100 | 1113 | 14 | AGCAAGTGTGACAG | 1 | 1 | CTGTCACACTTGCT | 97 |
| 1136 | 1149 | 14 | GTGGTCAGCGGCCG | 1 | 1 | CGGCCGCTGACCAC | 98 |
| 1138 | 1151 | 14 | GGTCAGCGGCCGGG | 1 | 1 | CCCGGCCGCTGACC | 99 |
| 1139 | 1152 | 14 | GTCAGCGGCCGGGA | 1 | 1 | TCCCGGCCGCTGAC | 100 |
| 1140 | 1153 | 14 | TCAGCGGCCGGGAT | 1 | 1 | ATCCCGGCCGCTGA | 101 |
| 1223 | 1236 | 14 | AGCGGCACCCTCAT | 1 | 1 | ATGAGGGTGCCGCT | 102 |
| 1224 | 1237 | 14 | GCGGCACCCTCATA | 1 | 1 | TATGAGGGTGCCGC | 103 |
| 1227 | 1240 | 14 | GCACCCTCATAGGC | 1 | 1 | GCCTATGAGGGTGC | 104 |
| 1228 | 1241 | 14 | CACCCTCATAGGCC | 1 | 1 | GGCCTATGAGGGTG | 105 |
| 1230 | 1243 | 14 | CCCTCATAGGCCTG | 1 | 1 | CAGGCCTATGAGGG | 106 |
| 1231 | 1244 | 14 | CCTCATAGGCCTGG | 1 | 1 | CCAGGCCTATGAGG | 107 |
| 1232 | 1245 | 14 | CTCATAGGCCTGGA | 1 | 1 | TCCAGGCCTATGAG | 108 |
| 1233 | 1246 | 14 | TCATAGGCCTGGAG | 1 | 1 | CTCCAGGCCTATGA | 109 |
| 1234 | 1247 | 14 | CATAGGCCTGGAGT | 1 | 1 | ACTCCAGGCCTATG | 110 |
| 1235 | 1248 | 14 | ATAGGCCTGGAGTT | 1 | 1 | AACTCCAGGCCTAT | 111 |
| 1236 | 1249 | 14 | TAGGCCTGGAGTTT | 1 | 1 | AAACTCCAGGCCTA | 112 |
| 1237 | 1250 | 14 | AGGCCTGGAGTTTA | 1 | 1 | TAAACTCCAGGCCT | 113 |
| 1238 | 1251 | 14 | GGCCTGGAGTTTAT | 1 | 1 | ATAAACTCCAGGCC | 114 |
| 1239 | 1252 | 14 | GCCTGGAGTTTATT | 1 | 1 | AATAAACTCCAGGC | 115 |
| 1244 | 1257 | 14 | GAGTTTATTCGGAA | 1 | 1 | TTCCGAATAAACTC | 116 |
| 1388 | 1401 | 14 | AACTTCCGGGACGA | 1 | 1 | TCGTCCCGGAAGTT | 117 |
| 1403 | 1416 | 14 | GCCTGCCTCTACTC | 1 | 1 | GAGTAGAGGCAGGC | 118 |
| 1406 | 1419 | 14 | TGCCTCTACTCCCC | 1 | 1 | GGGGAGTAGAGGCA | 119 |
| 1409 | 1422 | 14 | CTCTACTCCCCAGC | 1 | 1 | GCTGGGGAGTAGAG | 120 |
| 1433 | 1446 | 14 | GAGGTCATCACAGT | 1 | 1 | ACTGTGATGACCTC | 121 |
| 1580 | 1593 | 14 | TCACAGAGTGGGAC | 1 | 1 | GTCCCACTCTGTGA | 122 |
| 1747 | 1760 | 14 | GACCCCCAACCTGG | 1 | 1 | CCAGGTTGGGGGTC | 123 |
| 1826 | 1839 | 14 | GCACACTCGGGGCC | 1 | 1 | GGCCCCGAGTGTGC | 124 |
| 1985 | 1998 | 14 | GGGGGTGAGGGTGT | 1 | 1 | ACACCCTCACCCCC | 125 |
| 1986 | 1999 | 14 | GGGGTGAGGGTGTC | 1 | 1 | GACACCCTCACCCC | 126 |
| 1988 | 2001 | 14 | GGTGAGGGTGTCTA | 1 | 1 | TAGACACCCTCACC | 127 |
| 2237 | 2250 | 14 | TGCTGCCATGCCCC | 1 | 1 | GGGGCATGGCAGCA | 128 |
| 2465 | 2478 | 14 | GCCATCTGCTGCCG | 1 | 1 | CGGCAGCAGATGGC | 129 |
| 2466 | 2479 | 14 | CCATCTGCTGCCGG | 1 | 1 | CCGGCAGCAGATGG | 130 |
| 2469 | 2482 | 14 | TCTGCTGCCGGAGC | 1 | 1 | GCTCCGGCAGCAGA | 131 |
| 2928 | 2941 | 14 | GGGCTCCCTGATTA | 1 | 1 | TAATCAGGGAGCCC | 132 |
| 2929 | 2942 | 14 | GGCTCCCTGATTAA | 1 | 1 | TTAATCAGGGAGCC | 133 |
| 843 | 856 | 14 | TGAAGTTGCCCCAT | 1 | 1 | ATGGGGCAACTTCA | 134 |
| 844 | 857 | 14 | GAAGTTGCCCCATG | 1 | 1 | CATGGGGCAACTTC | 135 |
| 976 | 989 | 14 | GGTGGAGGTGTATC | 1 | 1 | GATACACCTCCACC | 136 |
| 977 | 990 | 14 | GTGGAGGTGTATCT | 1 | 1 | AGATACACCTCCAC | 137 |
| 978 | 991 | 14 | TGGAGGTGTATCTC | 1 | 1 | GAGATACACCTCCA | 138 |
| 1083 | 1096 | 14 | GCTTCCACAGACAG | 1 | 2 | CTGTCTGTGGAAGC | 139 |
| 1085 | 1098 | 14 | TTCCACAGACAGGC | 1 | 2 | GCCTGTCTGTGGAA | 140 |
| 1601 | 1614 | 14 | GCTGCTGCCCACGT | 1 | 2 | ACGTGGGCAGCAGC | 141 |
| 1721 | 1734 | 14 | TGGTTCCCTGAGGA | 1 | 2 | TCCTCAGGGAACCA | 142 |
| 2234 | 2247 | 14 | TCCTGCTGCCATGC | 1 | 2 | GCATGGCAGCAGGA | 143 |
| 2468 | 2481 | 14 | ATCTGCTGCCGGAG | 1 | 2 | CTCCGGCAGCAGAT | 144 |
| 1602 | 1615 | 14 | CTGCTGCCCACGTG | 1 | 3 | CACGTGGGCAGCAG | 145 |
| 1603 | 1616 | 14 | TGCTGCCCACGTGG | 1 | 3 | CCACGTGGGCAGCA | 146 |
| 1887 | 1900 | 14 | TGCTGAGCTGCTCC | 1 | 3 | GGAGCAGCTCAGCA | 147 |
| 1886 | 1899 | 14 | CTGCTGAGCTGCTC | 1 | 4 | GAGCAGCTCAGCAG | 148 |
| 1773 | 1786 | 14 | CCCCCAGCACCCAT | 1 | 5 | ATGGGTGCTGGGGG | 149 |
| 1137 | 1150 | 14 | TGGTCAGCGGCCGG | 2 | 1 | CCGGCCGCTGACCA | 150 |
| 1141 | 1154 | 14 | CAGCGGCCGGGATG | 2 | 1 | CATCCCGGCCGCTG | 151 |
| 1225 | 1238 | 14 | CGGCACCCTCATAG | 2 | 1 | CTATGAGGGTGCCG | 152 |
| 1226 | 1239 | 14 | GGCACCCTCATAGG | 2 | 1 | CCTATGAGGGTGCC | 153 |
| 1229 | 1242 | 14 | ACCCTCATAGGCCT | 2 | 1 | AGGCCTATGAGGGT | 154 |
| 1240 | 1253 | 14 | CCTGGAGTTTATTC | 2 | 1 | GAATAAACTCCAGG | 155 |
| 1241 | 1254 | 14 | CTGGAGTTTATTCG | 2 | 1 | CGAATAAACTCCAG | 156 |
| 1243 | 1256 | 14 | GGAGTTTATTCGGA | 2 | 1 | TCCGAATAAACTCC | 157 |
| 1483 | 1496 | 14 | GGGGACTTTGGGGA | 2 | 1 | TCCCCAAAGTCCCC | 158 |
| 1578 | 1591 | 14 | TGTCACAGAGTGGG | 2 | 1 | CCCACTCTGTGACA | 159 |
| 1683 | 1696 | 14 | TGATCCACTTCTCT | 2 | 1 | AGAGAAGTGGATCA | 160 |
| 1718 | 1731 | 14 | GCCTGGTTCCCTGA | 2 | 1 | TCAGGGAACCAGGC | 161 |
| 1748 | 1761 | 14 | ACCCCCAACCTGGT | 2 | 1 | ACCAGGTTGGGGGT | 162 |
| 1983 | 1996 | 14 | TTGGGGGTGAGGGT | 2 | 1 | ACCCTCACCCCCAA | 163 |
| 2086 | 2099 | 14 | TGTCCACTGCCACC | 2 | 1 | GGTGGCAGTGGACA | 164 |
| 2087 | 2100 | 14 | GTCCACTGCCACCA | 2 | 1 | TGGTGGCAGTGGAC | 165 |
| 2240 | 2253 | 14 | TGCCATGCCCCAGG | 2 | 1 | CCTGGGGCATGGCA | 166 |
| 3435 | 3448 | 14 | CTTTTGTAACTTGA | 2 | 1 | TCAAGTTACAAAAG | 167 |
| 742 | 755 | 14 | GGCTGCCCGCCGGG | 2 | 1 | CCCGGCGGGCAGCC | 168 |
| 845 | 858 | 14 | AAGTTGCCCCATGT | 2 | 1 | ACATGGGGCAACTT | 169 |
| 1205 | 1218 | 14 | CAAGGGAAGGGCAC | 2 | 2 | GTGCCCTTCCCTTG | 170 |
| 1242 | 1255 | 14 | TGGAGTTTATTCGG | 2 | 2 | CCGAATAAACTCCA | 171 |
| 1408 | 1421 | 14 | CCTCTACTCCCCAG | 2 | 2 | CTGGGGAGTAGAGG | 172 |
| 1579 | 1592 | 14 | GTCACAGAGTGGGA | 2 | 2 | TCCCACTCTGTGAC | 173 |
| 1599 | 1612 | 14 | AGGCTGCTGCCCAC | 2 | 2 | GTGGGCAGCAGCCT | 174 |
| 1682 | 1695 | 14 | CTGATCCACTTCTC | 2 | 2 | GAGAAGTGGATCAG | 175 |
| 1722 | 1735 | 14 | GGTTCCCTGAGGAC | 2 | 2 | GTCCTCAGGGAACC | 176 |
| 1746 | 1759 | 14 | TGACCCCCAACCTG | 2 | 2 | CAGGTTGGGGGTCA | 177 |
| 1982 | 1995 | 14 | TTTGGGGGTGAGGG | 2 | 2 | CCCTCACCCCCAAA | 178 |
| 2235 | 2248 | 14 | CCTGCTGCCATGCC | 2 | 2 | GGCATGGCAGCAGG | 179 |
| 2238 | 2251 | 14 | GCTGCCATGCCCCA | 2 | 2 | TGGGGCATGGCAGC | 180 |
| 2467 | 2480 | 14 | CATCTGCTGCCGGA | 2 | 2 | TCCGGCAGCAGATG | 181 |
| 3434 | 3447 | 14 | GCTTTTGTAACTTG | 2 | 2 | CAAGTTACAAAAGC | 182 |
| 890 | 903 | 14 | GCCCAGAGCATCCC | 2 | 2 | GGGATGCTCTGGGC | 183 |
| 905 | 918 | 14 | TGGAACCTGGAGCG | 2 | 2 | CGCTCCAGGTTCCA | 184 |
| 1597 | 1610 | 14 | ACAGGCTGCTGCCC | 2 | 3 | GGGCAGCAGCCTGT | 185 |
| 2233 | 2246 | 14 | TTCCTGCTGCCATG | 2 | 3 | CATGGCAGCAGGAA | 186 |
| 1774 | 1787 | 14 | CCCCAGCACCCATG | 2 | 7 | CATGGGTGCTGGGG | 187 |
| 1142 | 1155 | 14 | AGCGGCCGGGATGC | 3 | 1 | GCATCCCGGCCGCT | 188 |
| 1604 | 1617 | 14 | GCTGCCCACGTGGC | 3 | 1 | GCCACGTGGGCAGC | 189 |
| 1987 | 2000 | 14 | GGGTGAGGGTGTCT | 3 | 1 | AGACACCCTCACCC | 190 |
| 1082 | 1096 | 15 | CGCTTCCACAGACAG | 1 | 1 | CTGTCTGTGGAAGCG | 191 |
| 1083 | 1097 | 15 | GCTTCCACAGACAGG | 1 | 1 | CCTGTCTGTGGAAGC | 192 |
| 1084 | 1098 | 15 | CTTCCACAGACAGGC | 1 | 1 | GCCTGTCTGTGGAAG | 193 |
| 1136 | 1150 | 15 | GTGGTCAGCGGCCGG | 1 | 1 | CCGGCCGCTGACCAC | 194 |
| 1137 | 1151 | 15 | TGGTCAGCGGCCGGG | 1 | 1 | CCCGGCCGCTGACCA | 195 |
| 1138 | 1152 | 15 | GGTCAGCGGCCGGGA | 1 | 1 | TCCCGGCCGCTGACC | 196 |
| 1139 | 1153 | 15 | GTCAGCGGCCGGGAT | 1 | 1 | ATCCCGGCCGCTGAC | 197 |
| 1140 | 1154 | 15 | TCAGCGGCCGGGATG | 1 | 1 | CATCCCGGCCGCTGA | 198 |
| 1223 | 1237 | 15 | AGCGGCACCCTCATA | 1 | 1 | TATGAGGGTGCCGCT | 199 |
| 1224 | 1238 | 15 | GCGGCACCCTCATAG | 1 | 1 | CTATGAGGGTGCCGC | 200 |
| 1226 | 1240 | 15 | GGCACCCTCATAGGC | 1 | 1 | GCCTATGAGGGTGCC | 201 |
| 1227 | 1241 | 15 | GCACCCTCATAGGCC | 1 | 1 | GGCCTATGAGGGTGC | 202 |
| 1228 | 1242 | 15 | CACCCTCATAGGCCT | 1 | 1 | AGGCCTATGAGGGTG | 203 |
| 1229 | 1243 | 15 | ACCCTCATAGGCCTG | 1 | 1 | CAGGCCTATGAGGGT | 204 |
| 1230 | 1244 | 15 | CCCTCATAGGCCTGG | 1 | 1 | CCAGGCCTATGAGGG | 205 |
| 1231 | 1245 | 15 | CCTCATAGGCCTGGA | 1 | 1 | TCCAGGCCTATGAGG | 206 |
| 1232 | 1246 | 15 | CTCATAGGCCTGGAG | 1 | 1 | CTCCAGGCCTATGAG | 207 |
| 1233 | 1247 | 15 | TCATAGGCCTGGAGT | 1 | 1 | ACTCCAGGCCTATGA | 208 |
| 1234 | 1248 | 15 | CATAGGCCTGGAGTT | 1 | 1 | AACTCCAGGCCTATG | 209 |
| 1235 | 1249 | 15 | ATAGGCCTGGAGTTT | 1 | 1 | AAACTCCAGGCCTAT | 210 |
| 1236 | 1250 | 15 | TAGGCCTGGAGTTTA | 1 | 1 | TAAACTCCAGGCCTA | 211 |
| 1237 | 1251 | 15 | AGGCCTGGAGTTTAT | 1 | 1 | ATAAACTCCAGGCCT | 212 |
| 1238 | 1252 | 15 | GGCCTGGAGTTTATT | 1 | 1 | AATAAACTCCAGGCC | 213 |
| 1239 | 1253 | 15 | GCCTGGAGTTTATTC | 1 | 1 | GAATAAACTCCAGGC | 214 |
| 1240 | 1254 | 15 | CCTGGAGTTTATTCG | 1 | 1 | CGAATAAACTCCAGG | 215 |
| 1243 | 1257 | 15 | GGAGTTTATTCGGAA | 1 | 1 | TTCCGAATAAACTCC | 216 |
| 1403 | 1417 | 15 | GCCTGCCTCTACTCC | 1 | 1 | GGAGTAGAGGCAGGC | 217 |
| 1405 | 1419 | 15 | CTGCCTCTACTCCCC | 1 | 1 | GGGGAGTAGAGGCAG | 218 |
| 1406 | 1420 | 15 | TGCCTCTACTCCCCA | 1 | 1 | TGGGGAGTAGAGGCA | 219 |
| 1407 | 1421 | 15 | GCCTCTACTCCCCAG | 1 | 1 | CTGGGGAGTAGAGGC | 220 |
| 1408 | 1422 | 15 | CCTCTACTCCCCAGC | 1 | 1 | GCTGGGGAGTAGAGG | 221 |
| 1483 | 1497 | 15 | GGGGACTTTGGGGAC | 1 | 1 | GTCCCCAAAGTCCCC | 222 |
| 1579 | 1593 | 15 | GTCACAGAGTGGGAC | 1 | 1 | GTCCCACTCTGTGAC | 223 |
| 1603 | 1617 | 15 | TGCTGCCCACGTGGC | 1 | 1 | GCCACGTGGGCAGCA | 224 |
| 1682 | 1696 | 15 | CTGATCCACTTCTCT | 1 | 1 | AGAGAAGTGGATCAG | 225 |
| 1718 | 1732 | 15 | GCCTGGTTCCCTGAG | 1 | 1 | CTCAGGGAACCAGGC | 226 |
| 1721 | 1735 | 15 | TGGTTCCCTGAGGAC | 1 | 1 | GTCCTCAGGGAACCA | 227 |
| 1745 | 1759 | 15 | CTGACCCCCAACCTG | 1 | 1 | CAGGTTGGGGGTCAG | 228 |
| 1746 | 1760 | 15 | TGACCCCCAACCTGG | 1 | 1 | CCAGGTTGGGGGTCA | 229 |
| 1747 | 1761 | 15 | GACCCCCAACCTGGT | 1 | 1 | ACCAGGTTGGGGGTC | 230 |
| 1772 | 1786 | 15 | CCCCCCAGCACCCAT | 1 | 1 | ATGGGTGCTGGGGGG | 231 |
| 1887 | 1901 | 15 | TGCTGAGCTGCTCCA | 1 | 1 | TGGAGCAGCTCAGCA | 232 |
| 1982 | 1996 | 15 | TTTGGGGGTGAGGGT | 1 | 1 | ACCCTCACCCCCAAA | 233 |
| 1983 | 1997 | 15 | TTGGGGGTGAGGGTG | 1 | 1 | CACCCTCACCCCCAA | 234 |
| 1984 | 1998 | 15 | TGGGGGTGAGGGTGT | 1 | 1 | ACACCCTCACCCCCA | 235 |
| 1985 | 1999 | 15 | GGGGGTGAGGGTGTC | 1 | 1 | GACACCCTCACCCCC | 236 |
| 1986 | 2000 | 15 | GGGGTGAGGGTGTCT | 1 | 1 | AGACACCCTCACCCC | 237 |
| 1987 | 2001 | 15 | GGGTGAGGGTGTCTA | 1 | 1 | TAGACACCCTCACCC | 238 |
| 2233 | 2247 | 15 | TTCCTGCTGCCATGC | 1 | 1 | GCATGGCAGCAGGAA | 239 |
| 2234 | 2248 | 15 | TCCTGCTGCCATGCC | 1 | 1 | GGCATGGCAGCAGGA | 240 |
| 2236 | 2250 | 15 | CTGCTGCCATGCCCC | 1 | 1 | GGGGCATGGCAGCAG | 241 |
| 2237 | 2251 | 15 | TGCTGCCATGCCCCA | 1 | 1 | TGGGGCATGGCAGCA | 242 |
| 2238 | 2252 | 15 | GCTGCCATGCCCCAG | 1 | 1 | CTGGGGCATGGCAGC | 243 |
| 2464 | 2478 | 15 | TGCCATCTGCTGCCG | 1 | 1 | CGGCAGCAGATGGCA | 244 |
| 2465 | 2479 | 15 | GCCATCTGCTGCCGG | 1 | 1 | CCGGCAGCAGATGGC | 245 |
| 2466 | 2480 | 15 | CCATCTGCTGCCGGA | 1 | 1 | TCCGGCAGCAGATGG | 246 |
| 2467 | 2481 | 15 | CATCTGCTGCCGGAG | 1 | 1 | CTCCGGCAGCAGATG | 247 |
| 2468 | 2482 | 15 | ATCTGCTGCCGGAGC | 1 | 1 | GCTCCGGCAGCAGAT | 248 |
| 2469 | 2483 | 15 | TCTGCTGCCGGAGCC | 1 | 1 | GGCTCCGGCAGCAGA | 249 |
| 2928 | 2942 | 15 | GGGCTCCCTGATTAA | 1 | 1 | TTAATCAGGGAGCCC | 250 |
| 3434 | 3448 | 15 | GCTTTTGTAACTTGA | 1 | 1 | TCAAGTTACAAAAGC | 251 |
| 843 | 857 | 15 | TGAAGTTGCCCCATG | 1 | 1 | CATGGGGCAACTTCA | 252 |
| 844 | 858 | 15 | GAAGTTGCCCCATGT | 1 | 1 | ACATGGGGCAACTTC | 253 |
| 976 | 990 | 15 | GGTGGAGGTGTATCT | 1 | 1 | AGATACACCTCCACC | 254 |
| 977 | 991 | 15 | GTGGAGGTGTATCTC | 1 | 1 | GAGATACACCTCCAC | 255 |
| 1597 | 1611 | 15 | ACAGGCTGCTGCCCA | 1 | 2 | TGGGCAGCAGCCTGT | 256 |
| 1600 | 1614 | 15 | GGCTGCTGCCCACGT | 1 | 2 | ACGTGGGCAGCAGCC | 257 |
| 1601 | 1615 | 15 | GCTGCTGCCCACGTG | 1 | 2 | CACGTGGGCAGCAGC | 258 |
| 1720 | 1734 | 15 | CTGGTTCCCTGAGGA | 1 | 2 | TCCTCAGGGAACCAG | 259 |
| 1773 | 1787 | 15 | CCCCCAGCACCCATG | 1 | 2 | CATGGGTGCTGGGGG | 260 |
| 1883 | 1897 | 15 | GAGCTGCTGAGCTGC | 1 | 2 | GCAGCTCAGCAGCTC | 261 |
| 1885 | 1899 | 15 | GCTGCTGAGCTGCTC | 1 | 2 | GAGCAGCTCAGCAGC | 262 |
| 1886 | 1900 | 15 | CTGCTGAGCTGCTCC | 1 | 2 | GGAGCAGCTCAGCAG | 263 |
| 1602 | 1616 | 15 | CTGCTGCCCACGTGG | 1 | 3 | CCACGTGGGCAGCAG | 264 |
| 1725 | 1739 | 15 | TCCCTGAGGACCAGC | 1 | 3 | GCTGGTCCTCAGGGA | 265 |
| 1771 | 1785 | 15 | GCCCCCCAGCACCCA | 1 | 3 | TGGGTGCTGGGGGGC | 266 |
| 1120 | 1134 | 15 | CACCCACCTGGCAGG | 2 | 1 | CCTGCCAGGTGGGTG | 267 |
| 1141 | 1155 | 15 | CAGCGGCCGGGATGC | 2 | 1 | GCATCCCGGCCGCTG | 268 |
| 1225 | 1239 | 15 | CGGCACCCTCATAGG | 2 | 1 | CCTATGAGGGTGCCG | 269 |
| 1241 | 1255 | 15 | CTGGAGTTTATTCGG | 2 | 1 | CCGAATAAACTCCAG | 270 |
| 1242 | 1256 | 15 | TGGAGTTTATTCGGA | 2 | 1 | TCCGAATAAACTCCA | 271 |
| 1482 | 1496 | 15 | TGGGGACTTTGGGGA | 2 | 1 | TCCCCAAAGTCCCCA | 272 |
| 1578 | 1592 | 15 | TGTCACAGAGTGGGA | 2 | 1 | TCCCACTCTGTGACA | 273 |
| 1595 | 1609 | 15 | TCACAGGCTGCTGCC | 2 | 1 | GGCAGCAGCCTGTGA | 274 |
| 1599 | 1613 | 15 | AGGCTGCTGCCCACG | 2 | 1 | CGTGGGCAGCAGCCT | 275 |
| 1717 | 1731 | 15 | GGCCTGGTTCCCTGA | 2 | 1 | TCAGGGAACCAGGCC | 276 |
| 1719 | 1733 | 15 | CCTGGTTCCCTGAGG | 2 | 1 | CCTCAGGGAACCAGG | 277 |
| 1748 | 1762 | 15 | ACCCCCAACCTGGTG | 2 | 1 | CACCAGGTTGGGGGT | 278 |
| 2086 | 2100 | 15 | TGTCCACTGCCACCA | 2 | 1 | TGGTGGCAGTGGACA | 279 |
| 2232 | 2246 | 15 | CTTCCTGCTGCCATG | 2 | 1 | CATGGCAGCAGGAAG | 280 |
| 2235 | 2249 | 15 | CCTGCTGCCATGCCC | 2 | 1 | GGGCATGGCAGCAGG | 281 |
| 2239 | 2253 | 15 | CTGCCATGCCCCAGG | 2 | 1 | CCTGGGGCATGGCAG | 282 |
| 2472 | 2486 | 15 | GCTGCCGGAGCCGGC | 2 | 1 | GCCGGCTCCGGCAGC | 283 |
| 742 | 756 | 15 | GGCTGCCCGCCGGGG | 2 | 1 | CCCCGGCGGGCAGCC | 284 |
| 1119 | 1133 | 15 | GCACCCACCTGGCAG | 2 | 2 | CTGCCAGGTGGGTGC | 285 |
| 1596 | 1610 | 15 | CACAGGCTGCTGCCC | 2 | 2 | GGGCAGCAGCCTGTG | 286 |
| 1598 | 1612 | 15 | CAGGCTGCTGCCCAC | 2 | 2 | GTGGGCAGCAGCCTG | 287 |
| 1722 | 1736 | 15 | GGTTCCCTGAGGACC | 2 | 2 | GGTCCTCAGGGAACC | 288 |
| 1723 | 1737 | 15 | GTTCCCTGAGGACCA | 2 | 2 | TGGTCCTCAGGGAAC | 289 |
| 1750 | 1764 | 15 | CCCCAACCTGGTGGC | 2 | 2 | GCCACCAGGTTGGGG | 290 |
| 1882 | 1896 | 15 | GGAGCTGCTGAGCTG | 2 | 2 | CAGCTCAGCAGCTCC | 291 |
| 2115 | 2129 | 15 | TCACAGGCTGCAGCT | 2 | 2 | AGCTGCAGCCTGTGA | 292 |
| 2471 | 2485 | 15 | TGCTGCCGGAGCCGG | 2 | 2 | CCGGCTCCGGCAGCA | 293 |
| 3433 | 3447 | 15 | TGCTTTTGTAACTTG | 2 | 2 | CAAGTTACAAAAGCA | 294 |
| 1404 | 1418 | 15 | CCTGCCTCTACTCCC | 2 | 3 | GGGAGTAGAGGCAGG | 295 |
| 1884 | 1898 | 15 | AGCTGCTGAGCTGCT | 2 | 3 | AGCAGCTCAGCAGCT | 296 |
| 2231 | 2245 | 15 | GCTTCCTGCTGCCAT | 2 | 3 | ATGGCAGCAGGAAGC | 297 |
| 1118 | 1132 | 15 | GGCACCCACCTGGCA | 3 | 1 | TGCCAGGTGGGTGCC | 298 |
| 1082 | 1097 | 16 | CGCTTCCACAGACAGG | 1 | 1 | CCTGTCTGTGGAAGCG | 299 |
| 1083 | 1098 | 16 | GCTTCCACAGACAGGC | 1 | 1 | GCCTGTCTGTGGAAGC | 300 |
| 1136 | 1151 | 16 | GTGGTCAGCGGCCGGG | 1 | 1 | CCCGGCCGCTGACCAC | 301 |
| 1137 | 1152 | 16 | TGGTCAGCGGCCGGGA | 1 | 1 | TCCCGGCCGCTGACCA | 302 |
| 1138 | 1153 | 16 | GGTCAGCGGCCGGGAT | 1 | 1 | ATCCCGGCCGCTGACC | 303 |
| 1139 | 1154 | 16 | GTCAGCGGCCGGGATG | 1 | 1 | CATCCCGGCCGCTGAC | 304 |
| 1140 | 1155 | 16 | TCAGCGGCCGGGATGC | 1 | 1 | GCATCCCGGCCGCTGA | 305 |
| 1223 | 1238 | 16 | AGCGGCACCCTCATAG | 1 | 1 | CTATGAGGGTGCCGCT | 306 |
| 1224 | 1239 | 16 | GCGGCACCCTCATAGG | 1 | 1 | CCTATGAGGGTGCCGC | 307 |
| 1225 | 1240 | 16 | CGGCACCCTCATAGGC | 1 | 1 | GCCTATGAGGGTGCCG | 308 |
| 1226 | 1241 | 16 | GGCACCCTCATAGGCC | 1 | 1 | GGCCTATGAGGGTGCC | 309 |
| 1227 | 1242 | 16 | GCACCCTCATAGGCCT | 1 | 1 | AGGCCTATGAGGGTGC | 310 |
| 1228 | 1243 | 16 | CACCCTCATAGGCCTG | 1 | 1 | CAGGCCTATGAGGGTG | 311 |
| 1229 | 1244 | 16 | ACCCTCATAGGCCTGG | 1 | 1 | CCAGGCCTATGAGGGT | 312 |
| 1230 | 1245 | 16 | CCCTCATAGGCCTGGA | 1 | 1 | TCCAGGCCTATGAGGG | 313 |
| 1231 | 1246 | 16 | CCTCATAGGCCTGGAG | 1 | 1 | CTCCAGGCCTATGAGG | 314 |
| 1232 | 1247 | 16 | CTCATAGGCCTGGAGT | 1 | 1 | ACTCCAGGCCTATGAG | 315 |
| 1233 | 1248 | 16 | TCATAGGCCTGGAGTT | 1 | 1 | AACTCCAGGCCTATGA | 316 |
| 1234 | 1249 | 16 | CATAGGCCTGGAGTTT | 1 | 1 | AAACTCCAGGCCTATG | 317 |
| 1235 | 1250 | 16 | ATAGGCCTGGAGTTTA | 1 | 1 | TAAACTCCAGGCCTAT | 318 |
| 1236 | 1251 | 16 | TAGGCCTGGAGTTTAT | 1 | 1 | ATAAACTCCAGGCCTA | 319 |
| 1237 | 1252 | 16 | AGGCCTGGAGTTTATT | 1 | 1 | AATAAACTCCAGGCCT | 320 |
| 1238 | 1253 | 16 | GGCCTGGAGTTTATTC | 1 | 1 | GAATAAACTCCAGGCC | 321 |
| 1239 | 1254 | 16 | GCCTGGAGTTTATTCG | 1 | 1 | CGAATAAACTCCAGGC | 322 |
| 1240 | 1255 | 16 | CCTGGAGTTTATTCGG | 1 | 1 | CCGAATAAACTCCAGG | 323 |
| 1242 | 1257 | 16 | TGGAGTTTATTCGGAA | 1 | 1 | TTCCGAATAAACTCCA | 324 |
| 1403 | 1418 | 16 | GCCTGCCTCTACTCCC | 1 | 1 | GGGAGTAGAGGCAGGC | 325 |
| 1404 | 1419 | 16 | CCTGCCTCTACTCCCC | 1 | 1 | GGGGAGTAGAGGCAGG | 326 |
| 1405 | 1420 | 16 | CTGCCTCTACTCCCCA | 1 | 1 | TGGGGAGTAGAGGCAG | 327 |
| 1406 | 1421 | 16 | TGCCTCTACTCCCCAG | 1 | 1 | CTGGGGAGTAGAGGCA | 328 |
| 1407 | 1422 | 16 | GCCTCTACTCCCCAGC | 1 | 1 | GCTGGGGAGTAGAGGC | 329 |
| 1482 | 1497 | 16 | TGGGGACTTTGGGGAC | 1 | 1 | GTCCCCAAAGTCCCCA | 330 |
| 1578 | 1593 | 16 | TGTCACAGAGTGGGAC | 1 | 1 | GTCCCACTCTGTGACA | 331 |
| 1595 | 1610 | 16 | TCACAGGCTGCTGCCC | 1 | 1 | GGGCAGCAGCCTGTGA | 332 |
| 1596 | 1611 | 16 | CACAGGCTGCTGCCCA | 1 | 1 | TGGGCAGCAGCCTGTG | 333 |
| 1597 | 1612 | 16 | ACAGGCTGCTGCCCAC | 1 | 1 | GTGGGCAGCAGCCTGT | 334 |
| 1599 | 1614 | 16 | AGGCTGCTGCCCACGT | 1 | 1 | ACGTGGGCAGCAGCCT | 335 |
| 1602 | 1617 | 16 | CTGCTGCCCACGTGGC | 1 | 1 | GCCACGTGGGCAGCAG | 336 |
| 1717 | 1732 | 16 | GGCCTGGTTCCCTGAG | 1 | 1 | CTCAGGGAACCAGGCC | 337 |
| 1718 | 1733 | 16 | GCCTGGTTCCCTGAGG | 1 | 1 | CCTCAGGGAACCAGGC | 338 |
| 1719 | 1734 | 16 | CCTGGTTCCCTGAGGA | 1 | 1 | TCCTCAGGGAACCAGG | 339 |
| 1720 | 1735 | 16 | CTGGTTCCCTGAGGAC | 1 | 1 | GTCCTCAGGGAACCAG | 340 |
| 1721 | 1736 | 16 | TGGTTCCCTGAGGACC | 1 | 1 | GGTCCTCAGGGAACCA | 341 |
| 1724 | 1739 | 16 | TTCCCTGAGGACCAGC | 1 | 1 | GCTGGTCCTCAGGGAA | 342 |
| 1745 | 1760 | 16 | CTGACCCCCAACCTGG | 1 | 1 | CCAGGTTGGGGGTCAG | 343 |
| 1746 | 1761 | 16 | TGACCCCCAACCTGGT | 1 | 1 | ACCAGGTTGGGGGTCA | 344 |
| 1747 | 1762 | 16 | GACCCCCAACCTGGTG | 1 | 1 | CACCAGGTTGGGGGTC | 345 |
| 1748 | 1763 | 16 | ACCCCCAACCTGGTGG | 1 | 1 | CCACCAGGTTGGGGGT | 346 |
| 1770 | 1785 | 16 | TGCCCCCCAGCACCCA | 1 | 1 | TGGGTGCTGGGGGGCA | 347 |
| 1771 | 1786 | 16 | GCCCCCCAGCACCCAT | 1 | 1 | ATGGGTGCTGGGGGGC | 348 |
| 1772 | 1787 | 16 | CCCCCCAGCACCCATG | 1 | 1 | CATGGGTGCTGGGGGG | 349 |
| 1882 | 1897 | 16 | GGAGCTGCTGAGCTGC | 1 | 1 | GCAGCTCAGCAGCTCC | 350 |
| 1883 | 1898 | 16 | GAGCTGCTGAGCTGCT | 1 | 1 | AGCAGCTCAGCAGCTC | 351 |
| 1884 | 1899 | 16 | AGCTGCTGAGCTGCTC | 1 | 1 | GAGCAGCTCAGCAGCT | 352 |
| 1885 | 1900 | 16 | GCTGCTGAGCTGCTCC | 1 | 1 | GGAGCAGCTCAGCAGC | 353 |
| 1886 | 1901 | 16 | CTGCTGAGCTGCTCCA | 1 | 1 | TGGAGCAGCTCAGCAG | 354 |
| 1887 | 1902 | 16 | TGCTGAGCTGCTCCAG | 1 | 1 | CTGGAGCAGCTCAGCA | 355 |
| 1982 | 1997 | 16 | TTTGGGGGTGAGGGTG | 1 | 1 | CACCCTCACCCCCAAA | 356 |
| 1983 | 1998 | 16 | TTGGGGGTGAGGGTGT | 1 | 1 | ACACCCTCACCCCCAA | 357 |
| 1984 | 1999 | 16 | TGGGGGTGAGGGTGTC | 1 | 1 | GACACCCTCACCCCCA | 358 |
| 1985 | 2000 | 16 | GGGGGTGAGGGTGTCT | 1 | 1 | AGACACCCTCACCCCC | 359 |
| 1986 | 2001 | 16 | GGGGTGAGGGTGTCTA | 1 | 1 | TAGACACCCTCACCCC | 360 |
| 2231 | 2246 | 16 | GCTTCCTGCTGCCATG | 1 | 1 | CATGGCAGCAGGAAGC | 361 |
| 2232 | 2247 | 16 | CTTCCTGCTGCCATGC | 1 | 1 | GCATGGCAGCAGGAAG | 362 |
| 2233 | 2248 | 16 | TTCCTGCTGCCATGCC | 1 | 1 | GGCATGGCAGCAGGAA | 363 |
| 2234 | 2249 | 16 | TCCTGCTGCCATGCCC | 1 | 1 | GGGCATGGCAGCAGGA | 364 |
| 2235 | 2250 | 16 | CCTGCTGCCATGCCCC | 1 | 1 | GGGGCATGGCAGCAGG | 365 |
| 2236 | 2251 | 16 | CTGCTGCCATGCCCCA | 1 | 1 | TGGGGCATGGCAGCAG | 366 |
| 2237 | 2252 | 16 | TGCTGCCATGCCCCAG | 1 | 1 | CTGGGGCATGGCAGCA | 367 |
| 2238 | 2253 | 16 | GCTGCCATGCCCCAGG | 1 | 1 | CCTGGGGCATGGCAGC | 368 |
| 2464 | 2479 | 16 | TGCCATCTGCTGCCGG | 1 | 1 | CCGGCAGCAGATGGCA | 369 |
| 2465 | 2480 | 16 | GCCATCTGCTGCCGGA | 1 | 1 | TCCGGCAGCAGATGGC | 370 |
| 2466 | 2481 | 16 | CCATCTGCTGCCGGAG | 1 | 1 | CTCCGGCAGCAGATGG | 371 |
| 2467 | 2482 | 16 | CATCTGCTGCCGGAGC | 1 | 1 | GCTCCGGCAGCAGATG | 372 |
| 2468 | 2483 | 16 | ATCTGCTGCCGGAGCC | 1 | 1 | GGCTCCGGCAGCAGAT | 373 |
| 2469 | 2484 | 16 | TCTGCTGCCGGAGCCG | 1 | 1 | CGGCTCCGGCAGCAGA | 374 |
| 2471 | 2486 | 16 | TGCTGCCGGAGCCGGC | 1 | 1 | GCCGGCTCCGGCAGCA | 375 |
| 3432 | 3447 | 16 | TTGCTTTTGTAACTTG | 1 | 1 | CAAGTTACAAAAGCAA | 376 |
| 3433 | 3448 | 16 | TGCTTTTGTAACTTGA | 1 | 1 | TCAAGTTACAAAAGCA | 377 |
| 843 | 858 | 16 | TGAAGTTGCCCCATGT | 1 | 1 | ACATGGGGCAACTTCA | 378 |
| 976 | 991 | 16 | GGTGGAGGTGTATCTC | 1 | 1 | GAGATACACCTCCACC | 379 |
| 1600 | 1615 | 16 | GGCTGCTGCCCACGTG | 1 | 2 | CACGTGGGCAGCAGCC | 380 |
| 1601 | 1616 | 16 | GCTGCTGCCCACGTGG | 1 | 2 | CCACGTGGGCAGCAGC | 381 |
| 1722 | 1737 | 16 | GGTTCCCTGAGGACCA | 1 | 2 | TGGTCCTCAGGGAACC | 382 |
| 1118 | 1133 | 16 | GGCACCCACCTGGCAG | 2 | 1 | CTGCCAGGTGGGTGCC | 383 |
| 1119 | 1134 | 16 | GCACCCACCTGGCAGG | 2 | 1 | CCTGCCAGGTGGGTGC | 384 |
| 1241 | 1256 | 16 | CTGGAGTTTATTCGGA | 2 | 1 | TCCGAATAAACTCCAG | 385 |
| 1598 | 1613 | 16 | CAGGCTGCTGCCCACG | 2 | 1 | CGTGGGCAGCAGCCTG | 386 |
| 2114 | 2129 | 16 | CTCACAGGCTGCAGCT | 2 | 1 | AGCTGCAGCCTGTGAG | 387 |
| 2470 | 2485 | 16 | CTGCTGCCGGAGCCGG | 2 | 1 | CCGGCTCCGGCAGCAG | 388 |
| 1723 | 1738 | 16 | GTTCCCTGAGGACCAG | 2 | 2 | CTGGTCCTCAGGGAAC | 389 |
| 1749 | 1764 | 16 | CCCCCAACCTGGTGGC | 2 | 2 | GCCACCAGGTTGGGGG | 390 |
| 1750 | 1765 | 16 | CCCCAACCTGGTGGCC | 2 | 2 | GGCCACCAGGTTGGGG | 391 |
| 1880 | 1895 | 16 | GAGGAGCTGCTGAGCT | 2 | 2 | AGCTCAGCAGCTCCTC | 392 |
| 1881 | 1896 | 16 | AGGAGCTGCTGAGCTG | 2 | 2 | CAGCTCAGCAGCTCCT | 393 |

1. An oligomer of between 10-50 nucleobases in length which comprises a contiguous nucleobase sequence of a total of between 10-50 nucleobases, wherein said contiguous nucleobase sequence is at least 80% homologous to a corresponding region of a nucleic acid which encodes a mammalian PCSK9.

2. The oligomer according to claim 1, wherein the contiguous nucleobase sequence comprises no more than 3, such as no more than 2 mismatches to the corresponding region of a nucleic acid which encodes a mammalian PCSK9.

3. The oligomer according to claim 2, wherein said contiguous nucleobase sequence comprises no more than a single mismatch to the corresponding region of a nucleic acid which encodes a mammalian PCSK9.

4. The oligomer according to claim 3, wherein said contiguous nucleobase sequence comprises no mismatches, with the corresponding region of a nucleic acid which encodes a mammalian PCSK9.

5. The oligomer according to claim 1, wherein the nucleobase sequence of the oligomer consists of the contiguous nucleobase sequence.

6. The oligomer according to claim 1, wherein the nucleic acid which encodes a mammalian PCSK9 is selected from the group consisting of a nucleic acid which encodes a rodent PCSK9, and a non-human primate PCSK9.

7. The oligomer according to claim 1, wherein the nucleic acid which encodes a mammalian PCSK9 is the human PCSK9 nucleotide sequence SEQ ID No 2, or a naturally occurring allelic variant thereof.

8. The oligomer according to claim 1, wherein the contiguous nucleobase sequence is complementary to a corresponding region of both the human PCSK9 nucleic acid sequence and a non-human mammalian PCSK9 nucleic acid sequence.

9. The oligomer according to claim 1, wherein the contiguous nucleobase sequence is complementary to a corresponding region of both the human PCSK9 nucleic acid sequence, and the mouse PCSK9 nucleic acid sequence.

10. The oligomer according to claim 1, wherein the contiguous nucleobase sequence comprises a contiguous subsequence of at least 6, nucleobase residues which, when formed in a duplex with the complementary PCSK9 target RNA is capable of recruiting RNaseH.

11. The oligomer according to claim 10, wherein the contiguous nucleobase sequence comprises of a contiguous subsequence of at least 7, at least 8, at least 9 or at least 10 nucleobase residues which, when formed in a duplex with the complementary PCSK9 target RNA, is capable of recruiting RNaseH.

12. The oligomer according to claim 10 wherein said contiguous subsequence is at least 9 or at least 10 nucleobases in length, at least 12 nucleobases or at least 14 nucleobases in length, 14, 15 or 16 nucleobases residues which, when formed in a duplex with the complementary PCSK9 target RNA is capable of recruiting RNaseH.

13. The oligomer according to claim 1, wherein said oligomer is conjugated with one or more non-nucleobase compounds.

14. The oligomer according to claim 1, wherein said oligomer has a length of between 10-22 nucleobases.

15.-17. (canceled)

18. The oligomer according to claim 1 wherein said oligomer is single stranded.

19. The oligomer according to claim 1, wherein said contiguous nucleobase sequence comprises at least one affinity enhancing nucleotide analogue.

20. The oligomer according to claim 19, wherein said contiguous nucleobase sequence comprises a total of 2, 3, 4, 5, 6, 7, 8, 9 or 10 affinity enhancing nucleotide analogues.

21. The oligomer according to claim 1 which comprises at least one affinity enhancing nucleotide analogue, wherein the remaining nucleobases are selected from the group consisting of DNA nucleotides and RNA nucleotides.

22. The oligomer according to claim 1, wherein the oligomer comprises of a sequence of nucleobases of formula, in 5′ to 3′ direction, A-B-C, and optionally of formula A-B-C-D, wherein:
A. comprises of at least one nucleotide analogue;
B. comprises at least five consecutive nucleobases which are capable of recruiting RNAseH;
C. comprises of at least one nucleotide analogue;
D. comprises of one or more 1-3 or 1-2 DNA nucleotides.

23. The oligomer according to claim 22, wherein region A consists or comprises of 2, 3 or 4 consecutive nucleotide analogues.

24. The oligomer according to claim 22, wherein region B consists or comprises of 7, 8, 9 or 10 consecutive DNA nucleotides or equivalent nucleobases which are capable of recruiting RNAseH when formed in a duplex with a complementary RNA.

25. The oligomer according to claim 22, wherein region C comprises of 2, 3 or 4 consecutive nucleotide analogues.

26. The oligomer according to claim 22, wherein region D consists, where present, of one or two DNA nucleotides.

27. The oligomer according to claim 22, wherein:
A. comprises of 3 contiguous nucleotide analogues;
B. comprises of 7, 8, 9 or 10 contiguous DNA nucleotides or equivalent nucleobases which are capable of recruiting RNAseH when formed in a duplex with a complementary RNA;
C. comprises of 3 contiguous nucleotide analogues; and
D. Consists of one or two DNA nucleotides.

28. The oligomer according to claim 22, wherein the contiguous nucleobase sequence consists of 10, 11, 12, 13 or 14 nucleobases, and wherein;
A. Consists of 1, 2 or 3 contiguous nucleotide analogues;
B. Consists of 7, 8, or 9 consecutive DNA nucleotides or equivalent nucleobases which are capable of recruiting RNAseH when formed in a duplex with a complementary RNA;
C. Consists of 1, 2 or 3 contiguous nucleotide analogues; and
D. Consists of one DNA nucleotide.

29. The oligomer according to claim 22, wherein B comprises at least one LNA nucleobase which is in the alpha-L configuration.

30. The oligomer according to claim 1, wherein the nucleotide analogue(s) are independently or collectively selected from the group consisting of: Locked Nucleic Acid (LNA) units; 2′-O-alkyl-RNA units, 2′-OMe-RNA units, 2′-amino-DNA units, 2′-fluoro-DNA units, PNA units, HNA units, and INA units.

31. The oligomer according to claim 30 wherein all the nucleotide analogues(s) are LNA units.

32. The oligomer according to claim 1, which comprises 1, 2, 3, 4, 5, 6, 7. 8. 9 or 10 LNA units.

33. The oligomer according to claim 29, wherein the LNAs are independently selected from oxy-LNA, thio-LNA, and amino-LNA, in either of the beta-D and alpha-L configurations or combinations thereof.

34. The oligomer according to claim 33, wherein the LNAs are all β-D-oxy-LNA.

35. The oligomer according to claim 22, wherein the nucleotide analogues or nucleobases of regions A and C are β-D-oxy-LNA.

36. The oligomer according to claim 1, wherein at least one of the nucleobases present in the oligomers a modified nucleobase selected from the group consisting of 5-methylcytosine, isocytosine, pseudoisocytosine, 5-bromouracil, 5-propynyluracil, 6-aminopurine, 2-aminopurine, inosine, diaminopurine, and 2-chloro-6-aminopurine.

37. The oligomer according to claim 1, wherein said oligomer hybridises with a corresponding mammalian PCSK9 mRNA with a Tm of at least 40° C.

38. The oligomer according to claim 1, wherein said oligomer hybridises with a corresponding mammalian PCSK9 mRNA with a Tm of no greater than 80° C.

39. The oligomer according to claim 1, wherein the internucleoside linkages are independently selected from the group consisting of: phosphodiester, phosphorothioate and boranophosphate.

40. The oligomer according to claim 39, wherein the oligomer comprises at least one phosphorothioate internucleoside linkage.

41. The oligomer according to claim 40, wherein the internucleoside linkages adjacent to or between DNA or RNA units, or within region B are phosphorothioate linkages.

42. The oligomer according to claim 40, wherein the linkages between at least one pair of consecutive nucleotide analogues is a phosphodiester linkage.

43. The oligomer according to claim 40, wherein all the linkages between consecutive nucleotide analogues are phosphodiester linkages.

44. The oligomer according to claim 40 wherein all the internucleoside linkages are phosphorothioate linkages.

45. The oligomer according to claim 1, wherein said continuous nucleobase sequence corresponds to a contiguous nucleotide sequence present in a nucleic acid sequence selected from the group consisting of SEQ ID NO 14, SEQ ID NO 15, SEQ ID NO 16, SEQ ID NO 17, SEQ ID NO 18, and SEQ ED NO 19.

46. The oligomer according to claim 1, wherein said continuous nucleobase sequence is a contiguous nucleotide sequence present in a nucleic acid sequence selected from the group consisting of SEQ ID NO 40 to SEQ ID NO 393.

47. The oligomer according to claim 1, wherein said continuous nucleobase sequence is a contiguous nucleotide sequence present in a nucleic acid sequence selected from the group consisting of: SEQ ID NO 3, SEQ ID NO 4, SEQ ID NO 5, SEQ ID NO 6, SEQ ID NO 7, SEQ ID NO 8, SEQ ID NO 31, SEQ ID NO 32, SEQ ID NO 33, SEQ ID NO 34, SEQ ID NO 35, SEQ ID NO 36, SEQ ID NO 37, SEQ ID NO 38, and SEQ ID NO 39.

48. The oligomer according to claim 1, wherein said oligomer is selected from the group consisting of: SEQ ID NO 10, SEQ ID NO 20, SEQ ID NO 11, SEQ ID NO 9, SEQ ID NO 21, SEQ ID NO 22, SEQ ID NO 23, SEQ ID NO 24, SEQ ID NO 25, SEQ ID NO 26, SEQ ID NO 27, SEQ ID NO 28, and SEQ ID NO 29.

49. A conjugate comprising the oligomer according to claim 1 and at least one non-nucleotide or non-polynucleotide moiety covalently attached to said compound.

50. A pharmaceutical composition comprising an oligomer as defined in claim 1, and a pharmaceutically acceptable diluent, carrier, salt or adjuvant.

51. A pharmaceutical composition according to 50, wherein the oligomer is constituted as a pro-drug.

52. A pharmaceutical composition according to claim 50, which further comprises a therapeutic agent selected from the group consisting of: an Apo-B-100 (antisense) oligomer, a FABP4 (antisense) oligomer, a statin, a fibrate, a thioazolidinedione, an anti-inflamatory compound and an antiviral compound.

53. A method of treating a disease or disorder comprising administering an effective amount of the oligomer of claim 1, wherein said disease or disorder is selected from the group consisting of: hypercholesterolemia or related disorder, an inflammatory disease or disorder, arthritis, asthma alzheimer's disease, a metabolic disease or disorder, metabolic syndrome, diabetes and atherosclerosis.

54. (canceled)

55. A method for treating an inflammatory disorder comprising administering the pharmaceutical composition of claim 50 to a patient in need thereof.

56. A method for treating hypercholesterolemia, or related disorder, said method comprising administering the pharmaceutical composition of claim 50 to a patient in need thereof.

57. A method of reducing or inhibiting the expression of PCSK9 in a cell or a tissue, the method comprising the step of contacting said cell or tissue with the pharmaceutical composition of claim 50 so that expression of PCSK9 is reduced or inhibited.

58. A method of (i) reducing the level of blood serum cholesterol or ii) reducing the level of blood serum LDL-cholesterol, or iii) for improving the HDL/LDL ratio, in a patient, the method comprising the step of administering the pharmaceutical composition of claim 50 to the patient.

59. A method of lowering the plasma triglyceride in a patient, the method comprising the step of administering the pharmaceutical composition of claim 50 to the patient so that the blood serum triglyceride level is reduced.

60. A method of treating obesity in a patient, the method comprising the step of administering the pharmaceutical composition of claim 50 to the patient in need of treatment so that the body weight of the patient is reduced.

61. A method of treating insulin resistance in a patient, the method comprising the step of administering the pharmaceutical composition of claim 50 to the patient in need of treatment so that the patients sensitivity to insulin is increased.

62. A method of treating type II diabetes in a patient, the method comprising the step of administering the pharmaceutical composition of claim 50 to the patient.

63. A method for treating a metabolic disorder such as metabolic syndrome, diabetes or atherosclerosis, said method comprising administering the pharmaceutical composition of claim 50 to a patient in need thereof.
